(19) United States
(12) Patent Application Publication
(10) Pub. No.: US 2006/0020309 A1
(43) Pub. Date: Jan. 26, 2006

(54) Methods and products for producing lattices of EMR-treated islets in tissues, and uses therefor

(75) Inventors: Gregory B. Altshuler, Wilmington, MA (US); Ilya Yaroslavsky, North Andover, MA (US); Andrei V. Erofeev, North Andover, MA (US); Mikhail Z. Smirnov, Burlington, MA (US); James J. Childs, Franklin, MA (US)

Correspondence Address: WILMER CUTLER PICKERING HALE AND DORR LLP, 60 STATE STREET, BOSTON, MA, 02109, US

(73) Assignee: Palomar Medical Technologies, Inc., Burlington, MA (US)

(21) Appl. No.: 11/097,841

(22) Filed: Apr. 1, 2005

Related U.S. Application Data
(60) Provisional application No. 60/561,052, filed on Apr. 9, 2004.
(60) Provisional application No. 60/614,382, filed on Sep. 29, 2004.
(60) Provisional application No. 60/641,616, filed on Jan. 5, 2005.
(60) Provisional application No. 60/620,734, filed on Oct. 21, 2004.

(51) Int. Cl. A61N 5/06

(52) U.S. Cl. 607/88

(57) ABSTRACT

Methods of treatment of tissue with electromagnetic radiation (EMR) to produce lattices of EMR-treated islets in the tissue are disclosed. Also disclosed are devices and systems for producing lattices of EMR-treated islets in tissue, and cosmetic and medical applications of such devices and systems.

### RELATED APPLICATIONS

This application claims benefit of priority to U.S. Provisional Application No. 60/561,052, filed Apr. 9, 2004, U.S. Provisional Application No. 60/614,382, filed Sep. 29, 2004, and U.S. Provisional Application No. 60/641,616, filed Jan. 5, 2005; is a continuation-in-part of U.S. patent application Ser. No. 10/465,137, filed Jun. 19, 2003, which claims benefit of priority to U.S. Provisional Application No. 60/389,871, filed Jun. 19, 2002; is a continuation-in-part of U.S. patent application Ser. No. 10/033,302, filed Dec. 27, 2001, which claims benefit of priority to U.S. Provisional Application No. 60/258,855, filed Dec. 28, 2000; and is a continuation-in-part of U.S. patent application Ser. No. 10/080,652, filed Feb. 22, 2002, which claims priority to U.S. Provisional Application No. 60/272,745, filed Mar. 2, 2001.

### BACKGROUND OF THE INVENTION

1. Field of the Invention

The invention relates to the treatment of tissue with electromagnetic radiation (EMR) to produce lattices of EMR-treated islets in the tissue. The invention also relates to devices and systems for producing lattices of EMR-treated islets in tissue, and cosmetic and medical applications of such devices and systems.

2. Description of the Related Art

Electromagnetic radiation, particularly in the form of laser light, has been used in a variety of cosmetic and medical applications, including uses in dermatology, dentistry, ophthalmology, gynecology, otorhinolaryngology and internal medicine. For most dermatological applications, the EMR treatment can be performed with a device that delivers the EMR to the surface of the targeted tissues. For applications in internal medicine, the EMR treatment is typically performed with a device that works in combination with an endoscope or catheter to deliver the EMR to internal surfaces and tissues. As a general matter, the EMR treatment is typically designed to (a) deliver one or more particular wavelengths (or a particular continuous range of wavelengths) of EMR to a tissue to induce a particular chemical reaction, (b) deliver EMR energy to a tissue to cause an increase in temperature, or (c) deliver EMR energy to a tissue to damage or destroy cellular or extracellular structures.

Until recently, all photothermal applications of light in medicine have been based on one of three approaches. The first approach, known as the principle of selective photothermolysis, sets specific requirements for the wavelengths used (which need to be absorbed preferentially by chromophores in the target area) and for the duration of the optical pulse (which needs to be shorter than characteristic thermal relaxation time of the target area). This approach was later extended, and is often called the extended theory of selective photothermolysis, to encompass situations in which the target area and target chromophore are physically separated. The second approach relies on heat diffusion from the target chromophore to the target area. The third approach relies on absorption by a chromophore which is substantially uniformly present in the tissue (e.g., water). In this last case, the damage zone can, in principle, be controlled by manipulating wavelength, fluence, incident beam size, pulse width, and cooling parameters. All three approaches have drawbacks, the most significant of which is the difficulty in eliminating unwanted side effects. Usually, primary absorption of optical energy by water causes bulk tissue damage.

Examples of typical applications in photodermatology include the treatment of dyschromia (skin tone) and skin remodeling. The standard approach to treating dyschromia uses selective absorption of light by melanin in a pigmented lesion or by hemoglobin in blood vessels. A number of lasers and spectrally filtered arc-discharge lamps have been used for such treatments. Usually, the endpoint of treatment is the coagulation of vessels and pigmented lesions. The thermal stress to these targets causes vessels to collapse and die, and pigmented lesions to crust over followed by sloughing-off of the dead skin. In both cases, the skin tone is improved and, as a side effect of such treatment, skin remodeling can occur as the thermal stress to tissues surrounding the blood vessels and pigmented lesions can stimulate new collagen production. These treatment applications are generally safe due to the limitation of the damage to small structures such as vessels and melanin-containing spots.

One problem with selective photothermolysis is that the wavelength selected for the radiation is generally dictated by the absorption characteristics of the chromophore and may not be optimal for other purposes. Skin is a scattering medium, but such scattering is far more pronounced at some wavelengths than at others. Unfortunately, wavelengths preferentially absorbed by melanin, for example, are also wavelengths at which substantial scattering occurs. This is also true for the wavelengths typically utilized for treating vascular lesions. Photon absorption in skin also varies over the optical wavelength band, and some wavelengths typically used in selective photothermolysis are wavelengths at which skin is highly absorbent. The fact that wavelengths typically utilized for selective photothermolysis are highly scattered and/or highly absorbed limits the ability to selectively target body components and, in particular, limits the depths at which treatments can be effectively and efficiently performed. Further, much of the energy applied to a target region is either scattered and does not reach the body component undergoing treatment, or is absorbed in overlying or surrounding tissue. This low efficiency for such treatments means that larger and more powerful EMR sources are required in order to achieve a desired therapeutic result. However, increasing power generally causes undesired and potentially dangerous heating of tissue. Thus, increasing efficacy often decreases safety, and additional cost and energy must be utilized to mitigate the effects of this undesired tissue heating by surface cooling or other suitable techniques. Heat management for the more powerful EMR source is also a problem, generally requiring expensive and bulky water circulation or other heat management mechanisms. A technique which permits efficacious power levels and minimizes undesired heating is therefore desirable.

Photodermal treatments are further complicated because chromophore concentrations in a target (e.g., melanin in hair follicles) varies significantly from target to target and from patient to patient, making it difficult to determine optimal, or even proper, parameters for effective treatment of a given target. High absorption by certain types of skin, for example dark skinned individuals or people with very tanned skin, often makes certain treatments difficult, or even impossible, to safely perform. A technique which permits all types and pigmentations of skin to be safely treated, preferably with little or no pain, and preferably using substantially the same parameters, is therefore desirable.

Absorption of optical energy by water is widely used in two approaches for skin remodeling: ablative skin resurfacing, typically performed with either CO₂ (10.6μ) or Er:YAG (2.94μ) lasers, and non-ablative skin remodeling using a combination of deep skin heating with light from Nd:YAG (1.34μ), Er:glass (1.56μ) or diode laser (1.44μ) and skin surface cooling for selective damage of sub-epidermal tissue. Nevertheless, in both cases, a healing response of the body is initiated as a result of the limited thermal damage, with the final outcome of new collagen formation and modification of the dermal collagen/elastin matrix. These changes manifest themselves in smoothing out rhytides and general improvement of skin appearance and texture (often referred to as “skin rejuvenation”). The principal difference between the two techniques is the region of body where damage is initiated. In the resurfacing approach, the full thickness of the epidermis and a portion of upper dermis are ablated and/or coagulated. In the non-ablative approach, the zone of coagulation is shifted deeper into the tissue, with the epidermis being left intact. In practice, this is achieved by using different wavelengths: very shallow-penetrating ones in the ablative techniques (absorption coefficients of ˜900 cm⁻¹ and ˜13000 cm⁻¹ for CO₂ and Er:YAG wavelengths, respectively) and deeper-penetrating ones in the non-ablative modalities (absorption coefficients between 5 and 25 cm⁻¹). In addition, contact or spray cooling is applied to skin surface in non-ablative techniques, providing thermal protection for the epidermis. Resurfacing techniques have demonstrated significantly higher clinical efficacy. One drawback, which severely limited popularity of this treatment in the recent years, is a prolonged post-operative period requiring continuous care. Non-ablative techniques offer considerably reduced risk of side effects and are much less demanding on post-operative care. However, clinical efficacy of the non-ablative procedure is often unsatisfactory. The reasons for such differences in the clinical outcomes of the two procedures are not completely understood. However, one possibility is that damage (or lack thereof) to the epidermis may be an important factor determining both safety and efficacy outcomes. Obviously, destruction of the protective outer epidermal barrier (in particular, the stratum corneum) in the course of ablative skin resurfacing increases chances of wound contamination and potential complications. At the same time, release of growth factors (in particular, TGF-α) by epidermal cells have been shown to play a crucial role in the wound healing process and, therefore, in the final skin remodeling. Clearly, this process does not occur if the epidermis is intact.

### SUMMARY OF THE INVENTION

The present invention depends, in part, upon the discovery that, when using electromagnetic radiation (EMR) to treat tissues, there are substantial advantages to producing lattices of EMR-treated islets in the tissue rather than large, continuous regions of EMR-treated tissue. The lattices are periodic patterns of islets in one, two or three dimensions in which the islets correspond to local maxima of EMR-treatment of tissue. The islets are separated from each other by non-treated tissue (or differently- or less-treated tissue). The EMR-treatment results in a lattice of EMR-treated islets which have been exposed to a particular wavelength or spectrum of EMR, and which is referred to herein as a lattice of “optical islets.” When the absorption of EMR energy results in significant temperature elevation in the EMR-treated islets, the lattice is referred to herein as a lattice of “thermal islets.” When an amount of energy is absorbed that is sufficient to significantly disrupt cellular or intercellular structures, the lattice is referred to herein as a lattice of “damage islets.” When an amount of energy (usually at a particular wavelength) sufficient to initiate a certain photochemical reaction is delivered, the lattice is referred to herein as a lattice of “photochemical islets.” By producing EMR-treated islets rather than continuous regions of EMR-treatment, more EMR energy can be delivered to an islet without producing a thermal islet or damage islet, and/or the risk of bulk tissue damage can be lowered.

Thus, in various aspects, the invention provides improved devices and systems for producing lattices of EMR-treated islets in tissues, and improved cosmetic and medical applications of such devices and systems

In one aspect, the invention provides methods for increasing the permeability of the stratum corneum of the skin of a subject to a compound by applying EMR radiation to the stratum corneum to produce a lattice of EMR-treated islets. In particular, the invention provides methods for increasing the permeability of the stratum corneum by treating the stratum corneum with an EMR-treatment device that produces a lattice of EMR-treated islets the stratum corneum, in which the lattice of EMR-treated islets is heated to a temperature sufficient to increase the permeability of the stratum corneum to the compound. In some embodiments, the is a therapeutic agent such as a hormone, a steroid, a non-steroidal anti-inflammatory drug, an anti-neoplastic agent, an antihistamine or an anesthetic agent. In specific embodiments, the therapeutic agent is insulin, estrogen, prednisolone, loteprednol, ketorolac, diclofenac, methotrexate, a histamine H1 antagonists, chlorpheniramine, pyrilamine, mepyramine, emedastine, levocabastine or lidocaine. In some embodiments, the compound is a cosmetic agent such as a pigment, reflective agent or photoprotectant. In general, the lattice of EMR-treated islets is heated to a temperature sufficient to at least partially melt a crystalline lipid extracellular matrix in the stratum corneum. In some embodiments, the increase in permeability is reversible. In some embodiments, the stratum corneum remains damaged until it is replaced by new growth.

In another aspect, the invention provides methods of transdermal delivery of a compound to a subject by treating a portion of the stratum corneum of the subject with an EMR-treatment device that produces a lattice of EMR-treated islets heated to a temperature sufficient to increase the permeability of the stratum corneum to the compound.

In some embodiments, the invention provides methods for increasing the permeability of the stratum corneum by using an EMR-treatment device that delivers EMR energy to endogenous chromophores (e.g., water, lipid, protein) in the tissue. In other embodiments, the EMR-treatment device delivers EMR energy to exogenous EMR-absorbing particles in contact with the tissue.

In another aspect, the invention provides methods for selectively damaging a portion of tissue in a subject by applying EMR radiation to produce a lattice of EMR-treated islets which absorb an amount of EMR sufficient to damage the tissue in the EMR-treated islets but not sufficient to cause bulk tissue damage. In some embodiments, the damage is coagulation or denaturation of intracellular or extracellular proteins in the EMR-treated islets. In other embodiments, the damage is killing of cells or ablation of tissue.

In another aspect, the invention provides methods of producing lattices of damage islets in a tissue in order to treat various pathological conditions of a tissue. For example, in some embodiments, a lattice of damage islets is produced to cause damage to tissues in a wart, a callus, a psoriasis plaque, a sebaceous gland (to treat acne), a sweat gland (to treat body odor), fat tissue, or cellulite.

In another aspect, the invention provides methods of reducing pigment in the skin of a subject by treating a portion of the skin with an EMR-treatment device that produces a lattice of EMR-treated islets in at least one volume of tissue containing the pigment, whereby the pigment is destroyed without killing cells including the pigment. In another aspect, the invention provides methods of reducing pigment in the skin of a subject by treating a portion of the skin with an EMR-treatment device that produces a lattice of EMR-treated islets in at least one volume of tissue containing the pigment, whereby cells including the pigment are destroyed. In any of these embodiments, the pigment can be present in a tattoo, port wine stain, birthmark, or freckle.

In another aspect, the invention provides methods for skin rejuvenation, skin texturing, hypertrophic scar removal, skin lifting, stretch mark removal, non-skin-surface texturing (e.g. lip augmentation), and improved wound and burn healing by treating a portion of tissue of a subject with an EMR-treatment device that produces a lattice of EMR-treated damage islets in a desired treatment area and thereby activates an natural healing and/or repair process which improves the desired tissue characteristic.

In another aspect, the invention provides methods for photodynamic therapy of a subject in need thereof, by treating a portion of tissue of the subject with an EMR-treatment device that produces a lattice of EMR-treated islets in a desired treatment area and activates a photodynamic agent present in the islets. In some embodiments, the photodynamic agent is administered to the subject prior to treatment. In some embodiments, the photodynamic agent is an antineoplastic agent or a psoralen.

In the various embodiments of the invention, the lattices of EMR-treated islets can include a multiplicity of islets in which each islet has a maximum dimension of 1 μm to 30 mm, 1 μm to 10 μm, μm to 100 μm, 100 μm to 1 mm, 1 mm to 10 mm, or greater. In addition, the lattices can have fill factors of 0.01-90%, 0.01-0.1%, 0.1-1%, 1-10%, 10-30%, 30-50%, or greater. In addition, the lattices of islets can have minimum depths from the surface of a tissue of 0-4 mm, 0-50 μm, 50-500 μm, or 500 μm-4 mm, as well as sub-ranges within these.

In the various embodiments of the invention, the lattices of EMR-treated islets can be heated to temperatures of 35-40° C., 40-50° C., 50-100° C., 100-200° C., or greater than 200° C. In some embodiments, the papillary dermis is not heated to a temperature above 40-43° C. to prevent pain. In some embodiments, the upper layers of the tissue are cooled to reduce heating of those layers and/or produce subsurface thermal or damage islets.

In another series of aspects, the invention provides devices and systems for practicing the methods of the invention.

This, in one aspect of the invention is an apparatus for performing a treatment on a target area of a patient's skin in order to create treatment islets. According to this aspect, the apparatus features a housing that defines a target treatment area on the patient's skin when placed in proximity to the patient's skin, and an LED or diode laser bar mounted within the housing. The LED or diode laser bar can be used to apply optical energy to the target area. The LED or diode laser bar includes multiple emitters of optical energy for creating treatment islets in the patient's skin. The emitters can be spaced apart by varying amounts. In one aspect, the emitters are spaced apart by about 50 to 900 μm. The width of the emitters can also vary. In some aspects, the widths are about 50 to 150 μm. In some aspects, the emitters can be within about 50 to 1000 microns of the patient's skin, allowing the emitters to create treatment islets. The emitters can emit light in a variety of wavelengths, including, for example, in the wavelength range of about 290 to 10,000 nm. The diode laser bar can include any number of emitters. Some embodiments use between 10 and 25 emitters. Other embodiments can include multiple LEDs or diode laser bars in a hand piece to form a stack.

The apparatus set forth above can also include a variety of other components, such as, for example, a cooling element or a heating element attached to the housing. A cooling element can be disposed between the diode laser bar and the patient's skin when in use to cool the patient's skin. A heating element, on the other hand, can heat the patient's skin. In both cases, the element can allow passage of at least a portion of the optical energy from the LED or diode laser bar. The cooling or heating element can be made from, for example, sapphire or diamond.

The apparatus set forth above can also include a motor to move the diode laser bar with respect to the housing. The apparatus can include circuitry to vary the control of the motor to move the diode laser bar or LED in a direction opposite to a direction of movement of the housing across the patient's skin.

The apparatus set forth above can, in some aspects, include a mechanism coupled to the emitters for creating treatment islets in the patient's skin. This mechanism can be, for example, a lens array. The mechanism can also be a bundle of optical fibers, wherein each fiber is connected to at least one emitter.

The apparatus set forth above can be, in some aspects, a hand held device. The hand held device can be a hand held dermatological device that includes, for instance, control switches and a button to activate the diode laser bar or LED. The hand held device can be a stand-alone device or can be a device that communicates via an umbilical cord with a base unit.

Another aspect of the invention is an apparatus for performing a treatment on a target area of a patient's skin by applying optical energy on the target area. According to this aspect, the apparatus includes an optical energy source, an applicator movable to a position proximate the target area of the patient's skin for applying optical energy to the target area, and one or more optical fibers for transmitting optical energy from the optical energy source to the applicator. The applicator can include a mechanism for delivering optical energy onto the target area in order to create islets of treatment. The mechanism can be, for example, a total internal reflection element. The optical energy source can be either a coherent or a non-coherent light source.

Another aspect of the invention is a handheld dermatological device. In this aspect, the device includes a housing capable of being manually manipulated to position a head portion of the housing in proximity to a person's skin, and a plurality of optical fibers within the housing to couple radiation from a radiation source through the hand piece to the person's skin. In this aspect, the optical fibers can be spaced apart to output radiation to create treatment islets.

Another embodiment of the invention can be an apparatus for treating skin that includes a speed sensor. In this aspect, the apparatus features a light emitting assembly for applying optical energy to the target area of the patient's skin, the light emitting assembly including a head portion movable across the target area of the patient's skin and an optical energy source for outputting optical energy from the light emitting assembly. The source is movably mounted relative to the head, and a sensor determines the speed of movement of the head portion across the target area of the patient's skin. The apparatus can include circuitry in communication with the sensor for controlling movement of the source relative to the head portion based on the speed of movement of the head portion across the target area of the patient's skin, such that islets of treatment are formed on the target area of the patient's skin. The circuitry, for instance, can control the movement of the source such that the source is moved in a direction generally opposite the direction of movement of the head portion from a first position in the head portion to a second position in the head portion at generally the same speed as the movement of the head portion, and when the source reaches the second position, it is returned to the first position. The source can, for instance, be mounted on a linear translator in the head portion. In some aspects, the sensor can be a capacitive imaging array or an optical encoder. The source can be either a coherent or a non-coherent light source.

According to another aspect of the invention, an apparatus for performing a treatment on a target area of a patient's skin can prevent the passage of light to the patient's skin if the apparatus is not in contact with the patient's skin. Such an apparatus can feature a light emitting assembly including a non-coherent light source for applying optical energy to the target area and a plurality of light directing elements at an output end of the light emitting assembly. The light directing elements can be shaped so that substantially no light will pass through the output end when the output end is not in contact with the patient's skin. Further, the light directing elements can create treatment islets in the patient's skin during use. The light directing elements can be, for example, selected from a group including an array of pyramids, cones, hemispheres, grooves, and prisms.

According to another aspect of the invention, an apparatus for performing a treatment on a target area of a patient's skin can feature a light emitting assembly including a non-coherent light source for applying optical energy to the target area, and an element at an output end of the light emitting assembly that includes an optically diffusive surface with optically transmissive spots for output light spatial modulation. The optically transmissive spots can be one or more of circles, slits, rectangles, ovals, or irregular shapes.

Another aspect of the invention is a light emitting assembly for use in performing a treatment on a target area of a patient's skin. According to this aspect, the light emitting assembly includes a non-coherent light source and a light guide for transmitting optical energy from the light source to the target area. The light guide can include a bundle of optical fibers, with the bundle of optical fibers creating islets of treatment on the patient's skin during use. The fibers can be, for instance, spaced apart at an output of the light emitting assembly in order to create the treatment islets. Further, a micro-lens can be attached to an output end of the light guide to focus and/or modulate the light. The light source can be, for example, a linear flash lamp, an arc lamp, an incandescent lamp, or a halogen lamp.

Another aspect of the invention features a light emitting assembly that includes a plurality of non-coherent light sources and a plurality of light guides. Each light guide can transmit optical energy from a different one of the light sources to the target area of the patient's skin. In this aspect, the plurality of light guides provide light spatial modulation. The output ends of the light guides can be used to create islets of treatment on the patient's skin. In this aspect, the light source can be a linear flash lamp, an arc lamp, an incandescent lamp, or a halogen lamp.

Another aspect of the invention is an apparatus for performing a treatment on a target area of a patient's skin that includes a light emitting assembly and a mask. The light emitting assembly is for applying optical energy from an optical energy source to the target area of the patient's skin. The mask is attached to the light emitting assembly, and the mask is positioned between the optical energy source and the target area when the apparatus is in use. The mask includes one or more dielectric layers with a plurality of openings therethrough for passage of optical energy from the optical energy source to the target area. The apparatus can therefore create treatment islets in the patient's skin. In this aspect, the dielectric layers can have a high reflectance over a spectral band emitted by the optical energy source. The openings in the mask can have various shapes or identical shapes. For instance, the openings can be lines, circles, slits, rectangles, ovals, or irregular shapes. In some aspects, the apparatus can include a cooling or a heating element for cooling or heating the mask during use. The optical energy can be over a wide wavelength band. In one embodiment, infrared light is used. The optical energy can be applied with a pulse width of 100 fsec to 1 sec.

In another aspect, a dermatological device can include a housing capable of being manually manipulated to position a head portion of the housing in proximity to a person's skin, a light path between an energy source and the head portion, and a mirror with holes in it. The mirror is within the light path and the holes allow for passage of optical energy from the energy source to the target treatment area. Such a device can be used to create treatment islets in the person's skin. The energy source can be within the device or in a separate unit.

In another aspect of the invention, an apparatus for performing a treatment on a target area of a patient's skin includes a light emitting assembly for applying optical energy to the target area and an element attached to the light emitting assembly. The element is disposed between the light emitting assembly and the target area of the patient's skin when the apparatus is in use, and the element includes a reflective material to reflect optical energy from the light emitting assembly back to the light emitting assembly and openings in the reflective material to allow passage therethrough of optical energy from the light emitting assembly.

According to another aspect of the invention, an apparatus can include a skin lifting implement or vacuum source. According to one aspect, such an apparatus features a skin lifting implement to lift and stretch the target area of the skin beneath the lifting implement and a light emitting assembly for applying optical energy to the target area. During use, the light emitting assembly can be oriented to emit light toward the patient's skin in order to treat the patient's skin. The light emitting assembly can, in one embodiment, create treatment islets in the patient's skin.

Another aspect of the invention is a method for performing a treatment on a target area of a patient's skin beneath a skin fold. According to this aspect, the method includes lifting the patient's skin to form a skin fold and applying light beams from generally opposite sides of said skin fold such that said light beams intersect at said target area of the patient's skin.

Another aspect of the invention is a composition for use in performing a treatment on a target area of a patient's skin. The composition can feature a material applicable selectively over portions of the target area of a patient's skin. The material can include an absorbing exogenous chromophore. Application of optical energy on the material can selectively heat the portions of the target area. In one aspect, the composition can include a high concentration of the chromophore so that treatment islets are created in the patient's skin. The chromophore can be dispersed within the composition so that only portions of the composition having the chromophore heat up upon the application of the optical energy.

Another aspect of the invention features a substance for use in performing a treatment on a target area of a patient's skin. The substance features a film applicable over the target area of a patient's skin and a composition containing an absorbing exogenous chromophore. The composition is selectively affixed to portions of the film so that application of optical energy on the composition selectively heats the portions of the target area adjacent the composition. The chromophore can be carbon, a metal, an organic dye, a non-organic pigment, or a fullerene. IN one aspect, the composition can be printed using a printing head on the patient's skin. The film can be, for example, an optically clear polymer.

Another aspect of the invention is a kit for use in performing a treatment on a target area of a patient's skin. The kit can include a material applicable selectively over portions of the target area of a patient's skin and a light emitting assembly for applying optical energy to the target area of the patient's skin. The material can include an absorbing exogenous chromophore. In this aspect, application of optical energy from the light emitting assembly on the material heats the exogenous chromophores to selectively heat portions of the target area of the patient's skin. In one aspect, the optical energy has one or more wavelength bands that match the absorption spectrum of the absorbing exogenous chromophore. The material can be, in some aspects, a patch or a lotion for application to the patient's skin.

Another aspect of the invention is a dermatological device that features a housing capable of being manually manipulated to position a head portion of the housing in proximity to a person's skin so that the head portion defines a target treatment area on the person's skin when in contact with the person's skin. The device also includes a substrate having a plurality of absorbing elements, where incident radiation from an energy source heats up the absorbing elements so that the absorbing elements create treatment islets in the stratum corneum of the person's skin. The substrate can be, for instance, a mask that blocks incident radiation in areas of the mask without the absorbing elements. The mask can be a contact plate that acts as a cooling plate in some embodiments. The absorbing elements can be a variety of materials, such as, for example, carbon or a metal.

Another aspect of the invention is a dermatological delivery device. According to this aspect, the device includes a substrate having a plurality of absorbing elements and a composition contained on at least one side of the substrate. Incident radiation from an energy source can heat up the absorbing elements so that the absorbing elements create treatment islets in the stratum corneum of a person's skin. After removal of the substrate, at least a substantial portion of the composition remains on the person's skin.

Another aspect of the invention is a light emitting assembly for use in performing a treatment on a target area of a patient's skin. According to this aspect, the assembly can features a solid state laser, a fiber bundle for receiving optical energy from the laser, and focusing optics at an output end of the fiber bundle for projecting optical energy from each fiber of the fiber bundle onto the target area. The fiber bundle can spatially modulate the optical energy from the laser to create islets of treatment on the patient's skin.

According to another aspect of the invention, a light emitting assembly for use in performing a treatment on a target area of a patient's skin includes a solid state laser, a phase mask including a plurality of openings for propagating emission from the laser, and focusing optics at an output end of the phase mask to provide light spatial modulation on the target area. The light emitting assembly can be used to create islets of treatment on the patient's skin.

Another aspect of the invention includes a light emitting assembly for use in performing a treatment on a target area of a patient's skin. The light emitting assembly can include a bundle of fiber lasers and focusing optics at an output end of the bundle to focus emission of each laser onto the target area. The bundle of fiber lasers and focusing optics can create islets of treatment on the patient's skin.

Another aspect of the invention is an apparatus for performing a treatment on a target area of a patient's skin that includes a light emitting assembly and a plurality of light directing elements. The light emitting assembly includes a light source for applying optical energy to the target area of the patient's skin. The light directing elements are positioned at an output end of the light emitting assembly for output light spatial modulation and concentration. The optical energy can be applied in a multitude of sub-areas, with a substantial portion of the target area between the sub-areas remaining unaffected. The light source is selected from a linear flash lamp, an arc lamp, an incandescent lamp, or a halogen lamp in one embodiment. In other embodiment, the light source can be a solid state laser, a fiber laser, and a dye laser. In one aspect, the light directing elements can be a reflector, a mask, or a light duct. In another aspect, the light directing elements can be a micro lens array, or an array of pyramids, cones, hemispheres, grooves, or prisms. In another aspect, the light direction elements are focusing optics at an output end of a fiber bundle for projecting optical energy from each fiber of the fiber bundle onto the target area.

### BRIEF DESCRIPTION OF THE DRAWINGS

The following drawings are illustrative of embodiments of the invention and are not meant to limit the scope of the invention as encompassed by the claims.

FIG. 1 is a diagram showing an exemplary cross-section of human skin.

FIG. 2 is a schematic diagram showing the layers of skin.

FIGS. 3A and 3B are semi-schematic perspective and side views respectively of a section of a patient's skin and of equipment positioned thereon for practicing one embodiment.

FIGS. 4A and 4B are top views of various matrix arrays of cylindrical lenses, some of which are suitable for providing a line focus for a plurality of target portions.

FIG. 5 is a side schematic view of some components that can be used in some aspects of the invention.

FIG. 6 is a side view of a hand piece that can be used in some aspects of the invention.

FIG. 7 is a perspective view of another embodiment of the invention.

FIG. 8 is a perspective view of yet another embodiment of the invention.

FIG. 9A is a side view of yet another embodiment of the invention.

FIGS. 9B to 9E are enlarged, side views of the distal end of the embodiment of FIG. 9A.

FIG. 10A is a side view of yet another embodiment of the invention.

FIGS. 10B and 10C are enlarged, side views of the distal end of the embodiment of FIG. 10A.

FIG. 11 is a side view of yet another embodiment of the invention.

FIG. 12A is a side view of an embodiment of the invention using a diode laser bar.

FIG. 12B is a perspective view of a diode laser bar that can be used in the embodiment of FIG. 12A.

FIG. 12C is a side view of yet another embodiment of the invention, which uses multiple diode laser bars.

FIG. 12D is a side view of yet another embodiment of the invention, which uses multiple diode laser bars.

FIG. 12E is a side view of yet another embodiment of the invention, which uses multiple optical fibers to couple optical energy.

FIG. 13A is a side view of another embodiment of the invention.

FIG. 13B is a perspective view of a light source and optical fiber that can be used along with the embodiment of FIG. 13A.

FIG. 13C is a side view of an embodiment of the invention using a fiber bundle.

FIG. 13D is a bottom view of the embodiment of FIG. 13C.

FIG. 13E is an enlarged, side view of a distal end of one of the embodiments of 13A-13D.

FIG. 14A is a side view of another embodiment of the invention, which uses a fiber bundle.

FIG. 14B is a side view of another embodiment of the invention, which uses a phase mask.

FIG. 14C is a side view of another embodiment of the invention, which uses multiple laser rods.

FIG. 15 is a bottom view of another embodiment of the invention, which uses one or more capacitive imaging arrays.

FIG. 16 is a side view of another embodiment of the invention, which uses a motor capable of moving a diode laser bar within a hand piece.

FIG. 17 is a top view of one embodiment of a diode laser bar.

FIG. 18 is a side cross-sectional view of the diode laser bar of FIG. 17.

FIGS. 19A-19C are top views of three optical systems involving arrays of optical elements suitable for use in delivering radiation in parallel to a plurality of target portions.

FIGS. 20A-21D are side views of various lens arrays suitable for delivering radiation in parallel to a plurality of target portions.

FIGS. 22A-22D are side views of Fresnel lens arrays suitable for delivering radiation in parallel to a plurality of target portions.

FIGS. 23A-23C are side views of holographic lens arrays suitable for use in delivering radiation in parallel to a plurality of target portions.

FIGS. 24A-24B are side views of gradient lens arrays suitable for use in delivering radiation in parallel to a plurality of target portions.

FIGS. 25A-25C are top views of various matrix arrays of cylindrical lenses, some of which are suitable for providing a line focus for a plurality of target portions.

FIGS. 26A-26D are cross-sectional or side views of one layer of a matrix cylindrical lens system suitable for delivering radiation in parallel to a plurality of target portions.

FIGS. 27A, 27B, and 27C are a perspective view and cross-sectional side views, respectively, of a two layer cylindrical lens array suitable for delivering radiation in parallel to a plurality of target portions.

FIGS. 28-31 are side views of various optical objective arrays suitable for use in concentrating radiation to one or more target portions.

FIGS. 32A-37 are side views of various deflector systems suitable for use with the arrays of FIGS. 10-13 to move to successive target portions.

FIGS. 38 and 39 are side views of two different variable focus optical system suitable for use in practicing the teachings of this invention.

FIG. 40 is a perspective view of another embodiment of the invention for creating treatment islets.

FIGS. 41A and 41B are side views of yet another embodiment of the invention.

FIGS. 42A and 42B are side and top view, respectively, of an embodiment of the invention having a skin lifting implement, such as a vacuum.

FIG. 43A is a side view of yet another embodiment of the invention.

FIG. 43B is an enlarged, side view of the distal end of the embodiment of FIG. 43A.

FIG. 43C is an enlarged, bottom view of the distal end of the embodiment of FIG. 43A.

FIG. 44 is a perspective view of another embodiment of the invention for creating treatment islets.

FIG. 45 is a perspective shot of two views of another embodiment of the invention for creating treatment islets.

FIG. 46 is a perspective view of another embodiment of the invention for creating treatment islets.

FIG. 47 is a side view of an embodiment of the invention using a film with active islets.

FIG. 48 is a perspective view of another embodiment of the invention for creating treatment islets.

FIGS. 49A to 51B are side views of various embodiments of the invention for creating treatment islets.

FIG. 52-62 are as described in the examples.

FIG. 63 is the four-layer model of skin used in the computational model described in Example 1.

FIG. 64 is the threshold fluence for skin damage at the depths of 0.25 mm (1), 0.5 mm (2), and 0.75 mm (3) in the adiabatic mode as a function of the wavelength.

FIG. 65 is the penetration depth of light inside the type II skin vs. the wavelength for a circular beam of diameter 0.1 mm striking the skin through sapphire.

FIG. 66 is the normalized irradiance on the beam axis as a function of skin depth for 800 (1), 1060 (2), 1200 (3), 1440 (4), 1560 (5), and 1700 (6) nm wavelengths.

FIG. 67 is the normalized irradiance on the beam axis as a function of depth for 1064 nm light focused to skin depths of 0.5 (1), 0.6 (2), 0.7 (3), and 1 (4) mm.

FIG. 68 is tissue irradiance vs. depth for the collimated beam of diameter 10 mm (1) and 0.1 mm (2) striking type II skin surface through sapphire at wavelength 1060 nm.

### DETAILED DESCRIPTION

The present invention depends, in part, upon the discovery that, when using electromagnetic radiation (EMR) to treat tissues, whether for purposes of photodynamic therapy, photobleaching, photobiomodulation, photobiostimulation, photobiosuspension, thermal stimulation, thermal coagulation, thermal ablation or other applications, there are substantial advantages to producing lattices of EMR-treated islets in the tissue rather than large, continuous regions of EMR-treated tissue. The EMR-treated tissues can be any tissues for which such treatment is useful and appropriate, including but not limited to dermal tissues, mucosal tissues (e.g., oral mucosa, gastrointestinal mucosa), ophthalmic tissues (e.g., retinal tissues), vaginal tissue and glandular tissues (e.g., prostate tissue).

The lattices are periodic patterns of islets in one, two or three dimensions in which the islets correspond to local maxima of EMR-treatment of tissue. The islets are separated from each other by non-treated tissue (or differently- or less-treated tissue). The EMR-treatment results in a lattice of EMR-treated islets which have been exposed to a particular wavelength or spectrum of EMR, and which is referred to herein as a lattice of “optical islets.” When the absorption of EMR energy results in significant temperature elevation in the EMR-treated islets, the lattice is referred to herein as a lattice of “thermal islets.” When an amount of energy is absorbed that is sufficient to significantly disrupt cellular or intercellular structures, the lattice is referred to herein as a lattice of “damage islets.” When an amount of energy (usually at a particular wavelength) sufficient to initiate a certain photochemical reaction is delivered, the lattice is referred to herein as a lattice of “photochemical islets.”

By producing EMR-treated islets rather than continuous regions of EMR-treatment, untreated regions (or differently- or less-treated regions) surrounding the islets can act as thermal energy sinks, reducing the elevation of temperature within the EMR-treated islets and/or allowing more EMR energy to be delivered to an islet without producing a thermal islet or damage islet and/or lowering the risk of bulk tissue damage. Moreover, with respect to damage islets, it should be noted that the regenerative and repair responses of the body occur at wound margins (i.e., the boundary surfaces between damaged and intact areas) and, therefore, healing of damaged tissues is more effective with smaller damage islets, for which the ratio of the wound margin to volume is greater.

As described more fully below, the percentage of tissue volume which is EMR-treated versus untreated (or differently- or less-treated) can determine whether optical islets become thermal islets, damage islets or photochemical islets. This percentage is referred to as the “fill factor”, and can be decreased by increasing the center-to-center distance(s) of islets of fixed volume(s), and/or decreasing the volume(s) of islets of fixed center-to-center distance(s).

Because untreated tissue volumes act as a thermal sink, these volumes can absorb energy from treated volumes without themselves becoming thermal or damage islets. Thus, a relatively low fill factor can allow for the delivery of high fluence energy to some volumes while preventing the development of bulk tissue damage. Finally, because the untreated tissue volumes act as a thermal sink, as the fill factor decreases, the likelihood of optical islets reaching critical temperatures to produce thermal islets or damage islets also decreases (even if the EMR power density and total exposure remain constant for the islet areas).

Finally, as described in detail below, the present invention also depends, in part, upon the application of discoveries relating to the EMR and thermal energy absorption, transfer, and dissipation properties of tissue. Based, in part, upon these discoveries, the invention provides improved devices and systems for producing lattices of EMR-treated islets in tissues, and improved cosmetic and medical applications of such devices and systems in dermatology, dentistry, ophthalmology, gynecology, otorhinolaryngology and internal medicine in combination with endoscope and catheter techniques. Although the devices, systems and methods of the invention are described in detail for dermatological applications, they can be used for treatment of any tissue surface or subsurface areas to which EMR can be delivered.

References and Definitions.

The patent, scientific and medical publications referred to herein establish knowledge that was available to those of ordinary skill in the art at the time the invention was made. The entire disclosures of the issued U.S. patents, published and pending patent applications, and other references cited herein are hereby incorporated by reference.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the subject matter which is the invention, the following definitions are provided for certain terms which are used in the specification and appended claims.

Numerical Ranges. As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous. Finally, the variable can take multiple values in the range, including any sub-range of values within the cited range.

Or. As used herein, unless specifically indicated otherwise, the word “or” is used in the inclusive sense of “and/or” and not the exclusive sense of “either/or.”

Skin Structure

Although the devices and systems of the invention, and the general methods of the invention, can be practiced with many tissues of the body, currently the most common applications of EMR-treatment to tissues are in the field of dermatology. Therefore, the structure of the skin, including its constituent tissues, is described below in some detail, and the remainder of the disclosure will use the skin as an example. In addition, certain applications will be described which are uniquely adapted to the skin (e.g., tattoo removal, permeation of the stratum corneum). It should be understood, however, that the general methods are applicable to other tissues, and that one of ordinary skill in the art can adapt the teachings of the disclosure to other organs and tissues with merely routine experimentation.

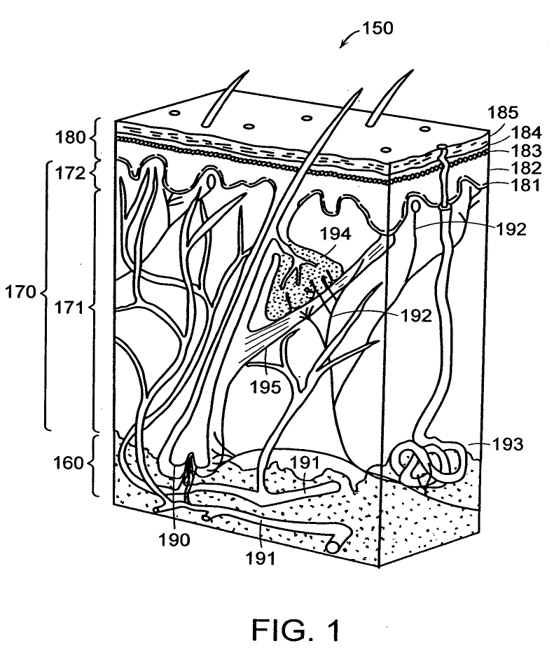

The skin is the largest organ in the human body, consisting of several layers of distinct tissues with distinct properties, and ranges in thickness from approximately 0.5 mm to approximately 4 mm. FIG. 1 illustrates a typical cross section of skin 150, showing various layers with differing cellular and intercellular structures.

The skin lies on top of the superficial fascia or subcutaneous tissue 160, a layer of fatty tissue that overlies the more densely fibrous fascia.

Above the subcutaneous tissue is the dermis 170, which comprises fibroelastic connective tissue, and ranges in thickness from approximately 0.3 mm on the eyelids to approximately 3.0 mm on the back. The dermis is highly vascularized and includes a variety of sensory nerves with temperature, pressure and pain receptors that are organized into small nerve bundles that ascend along with the blood vessels and lymphatic vessels to form a network of interlacing nerves beneath the epidermis, i.e., the superficial nerve plexus of the papillary dermis. Some of the nerves appear to penetrate the epidermis for short distances. The dermis includes two layers: a reticular layer 171 and a papillary layer 172. The reticular layer 171 includes cells in a matrix of dense, coarse bundles of collagenous fibers. The papillary layer 172 includes cells in a matrix of loose collagenous and elastic fibers, with elevations or papillae which project toward the epidermis. Cell types in the dermis include fibroblasts, mast cells and macrophages.

The epidermis 180 comprises the outermost stratified layers of the skin, and ranges in thickness from approximately 0.05 mm on the eyelids to approximately 1.5 mm on the palms and soles. The epidermis is avascular and consists largely of epithelial cells which mature as they pass from the innermost layer of columnar cells to the outermost layer of tile-like squamous cells, with the cells becoming increasingly flattened and keratinized as they progress outward. The innermost layer is referred to as the stratum basale, basal cell layer, or stratum germinativum 181, and is the only layer in normal epidermis in which cell division occurs. The next layer, the stratum spinosum 182, includes prickle cells and keratinocytes, and begins the production of keratin. The next layer, the stratum granulosum 183, is a darker layer with intercellular granules and increased keratin production. In thick skin, there is an additional transitional layer, the stratum lucidum 184. Finally, the outermost layer is the stratum corneum (SC) 185, a horny layer of highly keratinized squamous cells.

The cells of the stratum corneum 185 (and the stratum lucidum 184, when present) are highly keratinized (“horny”) and surrounded by an extracellular matrix consisting largely of crystalline lipids. As a result, the stratum corneum forms a hard, resilient barrier to water transport, and is not permeable to most aqueous or organic solvents or solutes. The stratum corneum 185 is about 15 μm deep on most anatomic sites but can be in the ranges of 10-300 μm (e.g., 20 μm at the forearm and 50-60 μm at the wrist).

Also shown are typical organs and structures within the skin, including a hair follicle 190, blood vessels 191, nerve fibers 192, a sweat gland 193, a sebaceous gland 194, and an arrector pili muscle 195.

Normal skin temperature is approximately 29-37° C. When exposed to temperatures in excess of 40-43° C., the sensory nerves of the dermis will transmit a pain response in most human subjects.

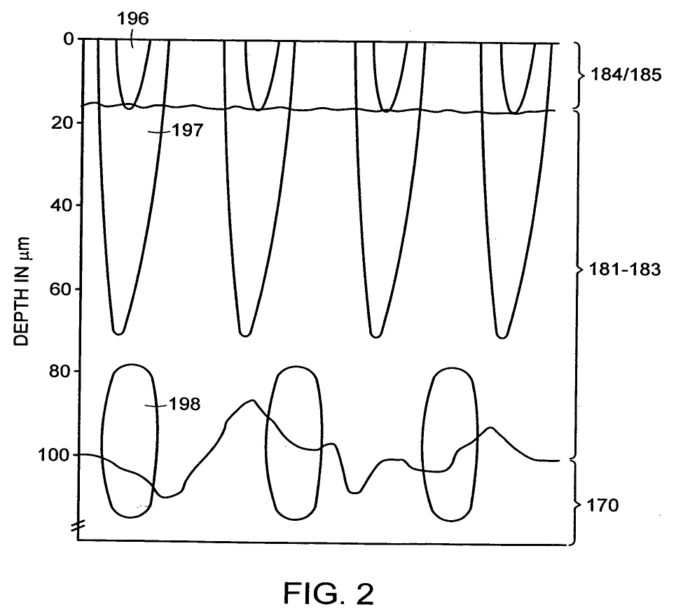

FIG. 2 is a schematic cross-sectional view of a human skin section 150. It shows depths into the skin, from the surface in μm. The stratum corneum 185 and stratum lucidum 184 are shown extending to a depth of approximately 15 μm below the skin surface. The remaining layers (i.e., layers 181-183) of the epidermis 180 extend from the stratum lucidum/corneum 184/185 to the boundary with the dermis 170 at a depth from the surface in the range of approximately 50-150 μm. Also shown are exemplary shallow islets 196 affecting the stratum lucidum/corneum 184/185, deeper islets 197 affecting the stratum lucidum/corneum 184/185 and deeper layers of the epidermis 180, and subsurface islets 198 spanning portions of the deeper epidermis 180 and upper dermis 170.

The depths shown in FIG. 2 are merely exemplary. Different locations in the typical human body have different depth profiles for the stratum corneum/lucidum, epidermis, and dermis. In addition, as described below. a great variety of other islet configurations are possible which are not shown in the figure (e.g., islets entirely in the dermis; islets entirely in the subcutaneous tissue; islets spanning the dermis and subcutaneous tissue; islets spanning portions of the epidermis, dermis and subcutaneous tissue).

Categories of EMR-Treated Islets

The present invention depends, in part, on the creation of a multiplicity of treated volumes of the skin which are separated by untreated volumes. The multiplicity of volumes can be described as defining a “lattice,” and the treated volumes, because they are separated by untreated volumes, can be described as “islets” within the skin. Depending upon the nature of the treatment, in particular the amount of energy transfer to the islets, the degree of heating of the tissue, or the wavelength(s) of the energy, four different categories of lattices can be produced: lattices of optical islets (LOI), lattices of thermal islets (LTI), lattices of damage islets (LDI), and lattices of photochemical islets (LPCI). These different categories of EMR-treated islets, devices and systems for producing such EMR-treated islets, and cosmetic and medical applications for such devices and systems are separately discussed in detail below. As used herein, the terms “treatment islet,” “islets of treatment,” and “EMR-treated islets” are used interchangeably to mean any of the categories of islets described below.

A. Optical Islets

In accordance with the present invention, EMR-treatment of completely or partially isolated volumes or islets of tissue produces a lattice of EMR-treated islets surrounded by untreated volumes. Although the islets can be treated with any form of EMR, they are referred to herein as “optical” islets for convenience, as many embodiments of the invention include the use of EMR within the ultraviolet, visible and infra-red spectrum. Other forms of EMR useful in the invention include microwave, radio frequency, low frequency and EMR induced by direct current.

As noted above, when the total energy transfer per unit cross-sectional area (i.e., fluence) or the rate of energy transfer per unit cross-sectional area (i.e., flux) becomes sufficiently high, the tissue of an optical islet will be heated, resulting in a thermal islet. If the temperature increase is sufficiently high, the tissue of a thermal islet will be damaged, resulting in a damage islet. Thus, although all thermal islets and damage islets are also optical islets, not all optical islets are thermal islets or damage islets. In some embodiments, as described below, it can be desirable to produce optical islets without producing thermal or damage islets. In such embodiments, the fill factor can be decreased in order to provide a greater volume of untreated tissue to act as a thermal sink.

As described in detail in the Examples below, a model of optical islets was developed which describes the propagation of light into skin taking into account the skin type and the characteristics of the light source. The particular approach used below is applicable to a wide range of islet dimensions (e.g., 10-30,000 μm in the lateral plane), is generally accepted in tissue optics, and is referred to as the light transport theory (Chandrasekhar (1960), Radiative Transfer (University Press, Oxford); Ishimaru (1978), Wave Propagation and Scattering in Random Media, Volume 1 (Academic Press, New York); Jacques et al.(1995), in Optical-Thermal Response of Laser-Irradiated Tissue, Welch et al., eds. (Plenum Press, New York), pp. 561-606). Briefly, the skin is considered as a multilayer structure with each layer being a turbid medium where light undergoes both absorption and multiple scattering. This approach neglects macroscopic coherence effects like diffraction and speckle formation. Several techniques may be used to solve the light transport problem in a tissue. Some of them, particularly the two-flux and diffusion approximations, break down when the input beam is sufficiently narrow or is focused into the tissue, and are not suitable for dealing with the islet formation problem. The Monte-Carlo technique described below is not subject to such limitations and is capable of modeling various tissue structures, spot profiles, wavelength spectra, and angular distributions of the incident light (Jacques et al. (1995), supra).

B. Thermal Islets

In accordance with another aspect of the present invention, EMR-treatment of isolated volumes or islets of tissue can produce a lattice of thermal islets with temperatures elevated relative to those of surrounding untreated volumes. Thermal islets result when energy is absorbed by an EMR-treated optical islet significantly faster than it is dissipated and, therefore, significant heating occurs.

Heating can result from the absorbance of EMR by water present throughout a volume of treated tissue, by endogenous chromophores present in selected cells or tissue(s) (e.g., melanin, hemoglobin), by exogenous chromophores within the tissue (e.g., tattoo ink) or, as described below, by exogenous chromophores applied to the surface of the tissue.

With respect to skin, in order to avoid causing pain to a subject, the maximal temperature of the basal membrane, which is adjacent to the nerve terminals of the papillary dermis, should not exceed 40-45° C. Assuming no active cooling of the skin surface, the temperature rise in the basal membrane, ΔT2, can be related to the temperature rise in the hyperthermic islets, ΔT1, by an approximate formula:
ΔT2=fΔT1
where f is the fill-factor of the optical lattice at the skin surface. This formula indicates that the temperature in the SC can attain relatively high values without triggering the pain response of the body if the fill factor is sufficiently low.

For example, setting ΔT2 to 12° C. and f to 0.3 yields ΔT1 of 40° C. In practice, the temperature rise ΔT1 may be limited by other factors, such as, for example, the threshold of structural damage to the SC or the desired size of the damage islets.

The thermal islet model is based, in part, on the time-dependent heat equation. Specifically, as described in more detail below, the thermal constants of the skin layers are obtained from Takata's relations (Takata et al. (1977), in Report SAM-TR-77-38 (San Antonio, Tex.: US Air Force School for Aerospace Medicine)) and are functions of the volume fraction of water in the corresponding layer. Specific effects associated with the bio-heat equation, e.g., the metabolic heat generation and the change of the blood perfusion rate while heating the living tissue, can be neglected for EMR pulses of short duration (Sekins et al. (1990), Thermal Science for Physical Medicine, in Therapeutic Heat and Cold, 4th edition, Lehmann, ed. (Baltimore: Williams & Wilkins) pp. 62-112). In practice, the EMR-heating can dominate strongly over metabolic heating and heat transfer by the blood flow. Moreover, the changes in the blood perfusion rate can occur with the delay of about 1 min with respect to the variations of the tissue temperature (Sekins et al. (1990), in Therapeutic Heat and Cold, 4th edition, Lehmann, ed. (Baltimore: Williams & Wilkins) pp. 62-112), and do not affect the islet formation dynamics unless tissues are under combined action (with EMR) of simultaneous physical factors (e.g., elevated or lowered external pressure, ultrasound, elevated or lowered skin surface temperature).

It should be emphasized that a lattice of thermal islets is a time-dependent phenomenon. If absorptive heating occurs at too great a rate or for too long a period, heat will begin to diffuse away from the EMR-treated islets and into the surrounding untreated tissue volumes. As this occurs, the thermal islets will spread into the untreated volumes and, ultimately, the thermal islets will merge and result in bulk heating. By using a sufficiently short pulse width relative to the temperature relaxation time of the target, it is possible to avoid merging or overlapping of thermal islets in a lattice.

C. Damage Islets

In accordance with another aspect of the present invention, EMR-treatment of isolated volumes or islets of tissue can produce a lattice of damage islets surrounded by volumes of undamaged tissue (or differently- or less-damaged tissue). Damage islets result when the temperature increase of an EMR-treated thermal islet is sufficient to result in protein coagulation, thermal injury, photodisruption, photoablation, or water vaporization. Depending upon the intended use, damage islets with lesser degrees of damage (e.g., protein coagulation, thermal injury) or greater degrees of damage (e.g., photodisruption, photoablation, or water vaporization) may be appropriate. As before, damage can result from the absorbance of EMR by water present throughout a volume of treated skin, by endogenous chromophores present in selected cells or tissue(s) in the skin (e.g., melanin, hemoglobin), by exogenous chromophores within the skin (e.g., tattoo ink) or, as described below, by exogenous chromophores applied to the surface of the skin.

As described in detail below, in some embodiments of the invention, the damage islets are thermal injuries with coagulation of structural proteins. Such damage can result when, for example, the light pulse duration varies from several microseconds to about 1 sec, but the peak tissue temperature remains below the vaporization threshold of water in the tissue (Pearce et al. (1995), in Optical-Thermal Response of Laser-Irradiated Tissue, Welch et al., eds. (Plenum Press, New York), pp. 561-606). The degree of damage is a function of the tissue temperature and the duration of the thermal pulse, and can be quantified by any of several damage functions known in the art. In the description below, for example, the damage function yielding the Arrhenius damage integral (Pearce et al. (1995), in Optical-Thermal Response of Laser-Irradiated Tissue, Welch et al., eds. (Plenum Press, New York), pp. 561-606; Henriques (1947), Arch. Pathol. 43:480-502) is employed. Other mechanisms and models of damage islet formation can apply to embodiments with relatively short and intense pulses, particularly in connection with photodisruption, photoablation, and water vaporization.

D. Photochemical Islets

In accordance with another aspect of the present invention, EMR-treatment of isolated volumes or islets of tissue can produce a lattice of photochemical islets surrounded by volumes of tissue in which a photochemical reaction has not been induced. The photochemical reaction can involve endogenous biomolecules or exogenous molecules. For example, exposure of the skin to certain wavelengths of EMR can result in increased melanin production and “tanning” through the activation of endogenous biomolecules and biological pathways. Alternatively, for example, exogenous molecules can be administered in photodynamic therapy, and activation of these molecules by certain wavelengths of EMR can cause a systemic or localized therapeutic effect.

Treatment Parameters.

In the practice of the invention, a variety of different treatment parameters relating to the applied EMR can be controlled and varied according to the particular cosmetic or medical application. These parameters include, without limitation, the following:

A. The Shape of EMR-Treated Islets.

The optical islets can be formed in any shape which can be produced by the devices described below, limited only by the ability to control EMR beams within the tissue. Thus, depending upon the wavelength(s), temporal characteristics (e.g., continuous versus pulsed delivery), and fluence of the EMR; the geometry, incidence and focusing of the EMR beam; and the index of refraction, absorption coefficient, scattering coefficient, anisotropy factor (the mean cosine of the scattering angle), and the configuration of the tissue layers; and the presence or absence of exogenous chromophores and other substances, the islets can be variously-shaped volumes extending from the surface of the skin through one or more layers, or extending from beneath the surface of the skin through one or more layers, or within a single layer. If the beams are not convergent, such beams will define volumes of substantially constant cross-sectional areas in the plane orthogonal to the beam axis (e.g., cylinders, rectanguloids). Alternatively, the beams can be convergent, defining volumes of decreasing cross-sectional area in the plane orthogonal to the central axis of the beams (e.g., cones, pyramids). The cross-sectional areas can be regular in shape (e.g., ellipses, polygons) or can be arbitrary in shape. In addition, depending upon the wavelength(s) and fluence of an EMR beam, and the absorption and scattering characteristics of a tissue for the wavelength(s), an EMR beam may penetrate to certain depths before being initially or completely absorbed or dissipated and, therefore, an EMR-treated islet may not extend through the entire depth of the skin but, rather, may extend between the surface and a particular depth, or between two depths below the surface.

Generally, the lattice is a periodic structure of islets in one, two, or three dimensions. For instance, a two-dimensional (2D) lattice is periodic in two dimensions and translation invariant or non-periodic in the third. The type of periodicity is characterized by the voxel shape. For example, and without limitation, there can be layer, square, hexagonal or rectangle lattices. The lattice dimensionality can be different from that of an individual islet. A single row of equally spaced infinite cylinders is an example of the 1D lattice of 2D islets (if the cylinders are of finite length this is the 1D lattice of 3D islets). The lattice dimensionality is equal to or smaller than the dimensionality of its islets (this fact follows from the fact that the lattice cannot be periodic in the dimension where its islets are translation invariant). Hence, there exists a total of 6 lattice types with each type being an allowed combination of the islet and lattice dimensionalities. For certain applications, an “inverted” lattice can be employed, in which islets of intact tissue are separated by areas of EMR-treated tissue and the treatment area is a continuous cluster of treated tissue with non treated islands.

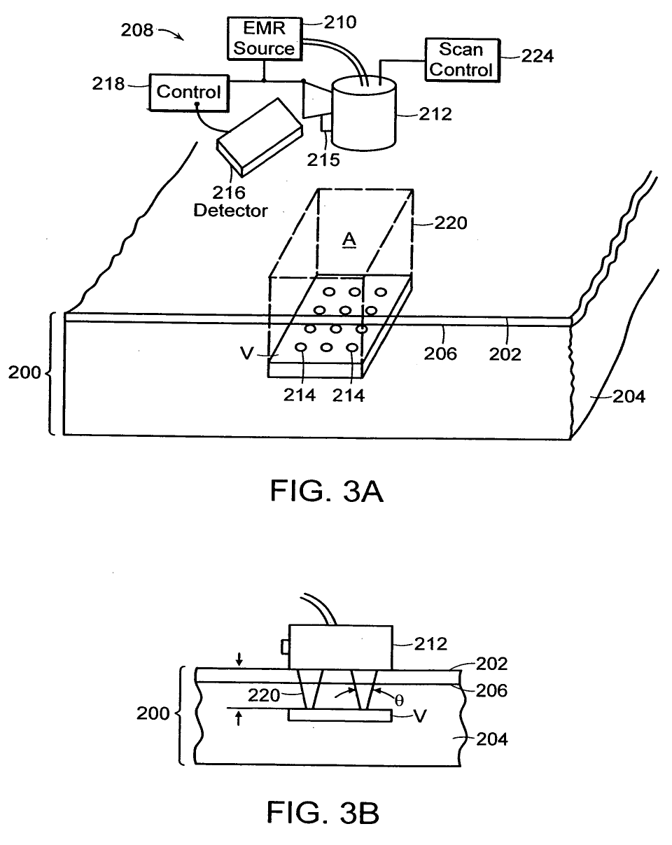

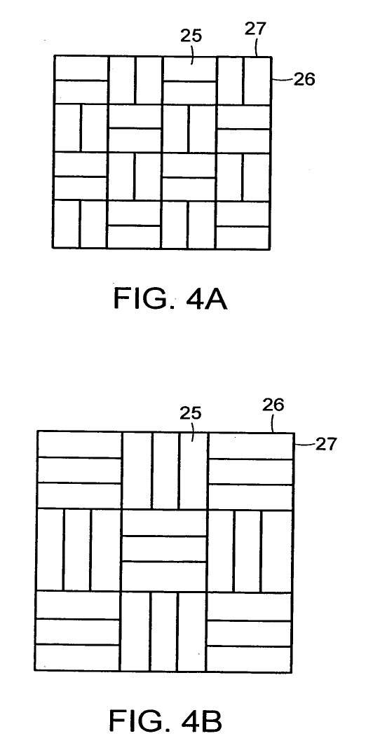

Referring to FIG. 3A, each of the treated volumes can be a relatively thin disk, as shown, a relatively elongated cylinder (e.g., extending from a first depth to a second depth), or a substantially linear volume having a length which substantially exceeds its width and depth, and which is oriented substantially parallel to the skin surface. The orientation of the lines for the islets 214 in a given application need not all be the same, and some of the lines may, for example, be at right angles to other lines (see for example FIGS. 4A and 4B). Lines also can be oriented around a treatment target for greater efficacy. For example, the lines can be perpendicular to a vessel or parallel to a wrinkle. Islets 214 can be subsurface volumes, such as spheres, ellipsoids, cubes or rectanguloids of selected thickness. The islets can also be substantially linear or planar volumes. The shapes of the islets are determined by the combined optical parameters of the beam, including beam size, amplitude and phase distribution, the duration of application and, to a lesser extent, the wavelength.

The parameters for obtaining a particular islet shape can be determined empirically with only routine experimentation. For example, a 1720 nm laser operating with a low conversion beam at approximately 0.005-2 J and a pulse width of 0.5-2 ms, can produce a generally cylindrically shaped islet. Alternatively, a 1200 nm laser operating with a highly converting beam at approximately 0.5-10 J and a pulse width of 0.5-3 sec, can produce a generally ellipsoid-shaped islet.

By suitable control of wavelength, focusing, incident beam size at the surface and other parameters, the islets, regardless of shape, can extend through a volume, can be formed in a single thin layer of a volume, or can be staggered such that adjacent islets are in different thin layers of volume. Most configurations of a lattice of islets can be formed either serially or in simultaneously. Lattices with islets in multiple thin layers in a volume can be easily formed serially, for example using a scanner or using multiple energy sources having different wavelengths. Islets in the same or varying depths can be created, and when viewed from the skin surface, the islets at varying depths can be either spatially separated or overlapping.

The geometry of the islets affects the thermal damage in the treatment region. Since a sphere provides the greatest gradient, and is thus the most spatially confined, it provides the most localized biological damage, and can therefore be preferred for applications where this is desirable.

B. The Size of EMR-Treated Islets.

The size of the individual islets within the lattices of EMR-treated islets of the invention, can vary widely depending upon the intended cosmetic or medical application. As discussed more fully below, in some embodiments it is desirable to cause substantial tissue damage to destroy a structure or region of tissue (e.g., a sebaceous gland, hair follicle, tattooed area) whereas in other embodiments it is desirable to cause little or no damage while administering an effective amount of EMR at a specified wavelength (e.g., photodynamic therapy). As noted above with respect to damage islets, however, the healing of damaged tissues is more effective with smaller damage islets, for which the ratio of the wound margin to volume is greater.

As a general matter, the size of the EMR-treated islets of the present invention can range from 1 μm to 30 mm in any particular dimension. For example, and without limitation, a lattice of substantially linear islets can consist of parallel islets have a length of approximately 30 mm and a width of approximately 10 μm to 1 mm. As another example, and without limitation, for substantially cylindrical islets in which the axis of the cylinder is orthogonal to the tissue surface, the depth can be approximately 10 μm to 4 mm and the diameter can be approximately 10 μm to 1 mm. For substantially spherical or ellipsoidal islets, the diameter or major axis can be, for example, and without limitation, approximately 10 μm to 1 mm. Thus, in some embodiments, the islets can have a maximum dimension in the range from 1 μm to 10 μm, 10 μm to 100 μm, 100 μm to 1 mm, 1 mm to 10 mm, or 10 mm to 30 mm, as well as all possible ranges within 1 μm to 30 mm.

When considering the size of the optical, thermal, damage or photochemical islets of the invention, it is important to note that the boundaries of the islets may not be clearly demarcated but, rather, may vary continuously or blend into the untreated tissue (or differently- or less-treated tissue). For example, EMR beams are subject to scattering in various tissues and, therefore, even beams of coherent light will become diffuse as they penetrate through multiple layers of cells or tissues. As a result, optical and photochemical islets typically will not have clear boundaries between treated and untreated volumes. Similarly, thermal islets typically will exhibit a temperature gradient from the center of the islet to its boundaries, and untreated tissue surrounding the islet also will exhibit a temperature gradient due to conduction of heat. Finally, damage islets can have irregular or indistinct boundaries due to partially damaged cells or structures or partially coagulated proteins. As used herein, therefore, the size of an islet within a lattice of islets, refers to the size of the islet as defined by the intended minimum or threshold amount of EMR energy delivered. As discussed in greater detail below, this amount is expressed as the minimum fluence, Fₘᵢₙ, and is determined by the nature of the cosmetic or medical application. For example, for photodynamic therapy, Fₘᵢₙ can be determined by the minimum fluence necessary to cause the desired photochemical reaction. Similarly, for increasing the permeability of the stratum corneum, Fₘᵢₙ can be determined by the minimum fluence necessary to achieve the desired SC temperature, and for destroying tissue, Fₘᵢₙ can be determined by the minimum fluence necessary to ablate the tissue or vaporize water. In each case, the size of the EMR-treated islet is defined by the size of the tissue volume receiving the desired minimum fluence.

Because of the scattering effects of tissue, the minimum size of an EMR-treated islet increases with the targeted depth in the tissue, ranging from several microns on the stratum corneum to several millimeters in subcutaneous tissue. For a depth of approximately 1 mm into a subject's tissue, the minimum diameter or width of an islet is estimated to be approximately 100 μm, although much larger islets (e.g., 1-10 mm) are possible. The size of a damage islet can be either smaller or larger than the size of the corresponding optical islet, but is generally larger as greater amounts of EMR energy are applied to the optical islet due to heat diffusion. For a minimum size islet at any particular depth in the skin, the wavelength, beam size, convergence, energy and pulse width have to be optimized.

C. The Depth of EMR-Treated Islets.

The EMR-treated islets of the invention can be located at varying points within a tissue, including surface and subsurface locations, locations at relatively limited depths, and locations spanning substantial depths. The desired depth of the islets depends upon the intended cosmetic or medical application, including the location of the targeted molecules, cells, tissues or intercellular structures.

For example, optical islets can be induced at varying depths in a tissue or organ, depending upon the depth of penetration of the EMR energy, which depends in part upon the wavelength(s) and beam size. Thus, the islets can be shallow islets that penetrate only surface layers of a tissue (e.g., 0-50 μm), deeper islets that span several layers of a tissue (e.g., 50-500 μm), or very deep, subsurface islets ((e.g., 500 μm-4 mm). Using optical energy, depths of up to 25 mm can be achieved using wavelengths of 1,000-1,300 nm. Using microwave and radio frequency EMR, depths of several centimeters can be achieved.

For thermal islets or damage islets, subsurface islets can be produced by targeting chromophores present only at the desired depth(s), or by cooling upper layers of a tissue while delivering EMR. For creating deep thermal or damage islets, long pulse widths coupled with surface cooling can be particularly effective.

D. Fill Factor of EMR-Treated Lattices

In a given lattice of EMR-treated islets, the percentage of tissue volume which is EMR-treated is referred to as the “fill factor” or f, and can affect whether optical islets become thermal islets, damage islets or photochemical islets. The fill factor is defined by the volume of the islets with respect to a reference volume that contains all of the islets. The fill factor may be uniform for a periodic lattice of uniformly sized EMR-treated islets, or it may vary over the treatment area. Non-uniform fill factors can be created in situations including, but not limited to, the creation of thermal islets using topical application of EMR-absorbing particles in a lotion or suspension (see below). For such situations, an average fill factor (f_{avg}) can be calculated by dividing the volume of all EMR-treated islets Vᵢ^{islet} by the volume of all tissue Vᵢ^{tissue} in the treatment area,

$${f}_{avg} = {\sum }_{i} \frac{{V}_{i}^{islet}}{{V}_{i}^{tissue}} .$$

Generally, the fill factor can be decreased by increasing the center-to-center distance(s) of islets of fixed volume(s), and/or decreasing the volume(s) of islets of fixed center-to-center distance(s). Thus, the calculation of the fill factor will depend on volume of an EMR-treated islet as well as on the spacing between the islets. In a periodic lattice, where the centers of the nearest islets are separated by a distance d, the fill factor will depend on the ratio of the size of the islet to the spacing between the nearest islets d. For example, in a lattice of parallel cylindrical islets, the fill factor will be:

$$f = π {( \frac{r}{d} )}^{2} ,$$

where d is the shortest distance between the centers of the nearest islets and r is the radius of a cylindrical EMR-treated islet. In a lattice of spherical islets, the fill factor will be the ratio of the volume of the spherical islet to the volume of the cube defined by the neighboring centers of the islets:

$$f = \frac{4 π}{3} {( \frac{r}{d} )}^{3} ,$$

where d is the shortest distance between the centers of the nearest islets and r is the radius of a spherical EMR-treated islet. Similar formulas can be obtained to calculate fill factors of lattices of islets of different shapes, such as lines, disks, ellipsoids, rectanguloids, or other shapes.

Because untreated tissue volumes act as a thermal sink, these volumes can absorb energy from treated volumes without themselves becoming thermal or damage islets. Thus, a relatively low fill factor can allow for the delivery of high fluence energy to some volumes while preventing the development of bulk tissue damage. Finally, because the untreated tissue volumes act as a thermal sink, as the fill factor decreases, the likelihood of optical islets reaching critical temperatures to produce thermal islets or damage islets also decreases (even if the EMR power density and total exposure remain constant for the islet areas).

The center-to-center spacing of islets is determined by a number of factors, including the size of the islets and the treatment being performed. Generally, it is desired that the spacing between adjacent islets be sufficient to protect the tissues and facilitate the healing of any damage thereto, while still permitting the desired therapeutic effect to be achieved. In general, the fill factor can vary in the range of 0.1-90%, with ranges of 0.1-1%, 1-10%, 10-30% and 30-50% for different applications. The interaction between the fill factor and the thermal relaxation time of a lattice of EMR-treated islets is discussed in detail below. In the case of lattices of thermal islets, it can be important that the fill factor be sufficiently low to prevent excessive heating and damage to islets, whereas with damage islets it can be important that the fill factor be sufficiently low to ensure that there is undamaged tissue around each of the damage islets sufficient to prevent bulk tissue damage and to permit the damaged volumes to heal.

Applications of EMR-Treated Islets

EMR-treated islets can be used in a variety of applications in a variety of different organs and tissues. For example, EMR treatments can be applied to tissues including, but not limited to, skin, mucosal tissues (e.g., oral mucosa, gastrointestinal mucosa), ophthalmic tissues (e.g., conjuctiva, cornea, retina), and glandular tissues (e.g., lacrimal, prostate glands). As a general matter, the methods can be used to treat conditions including, but not limited to, lesions (e.g., sores, ulcers), acne, rosacea, undesired hair, undesired blood vessels, hyperplastic growths (e.g., tumors, polyps, benign prostatic hyperplasia), hypertrophic growths (e.g., benign prostatic hypertrophy), neovascularization (e.g., tumor-associated angiogenesis), arterial or venous malformations (e.g., hemangiomas, nevus flammeus), and undesired pigmentation (e.g., pigmented birthmarks, tattoos).

A. Thermal Islets

In some aspects, the invention provides methods of treating tissues by creating lattices of thermal islets. These methods can be used in, for example, methods of increasing the permeability of the stratum corneum to various agents, including therapeutic agents and cosmetic agents, and methods for producing therapeutic hyperthermia. 1. Reversible Permeation of the Stratum Corneum

In one embodiment, lattices of thermal islets are produced in order to reversibly increase the permeability of the stratum corneum by heating islets of tissue to temperatures of 35-100° C. The increased permeability results from the melting of the extracellular matrix of crystalline lipids that surrounds the cells of the stratum corneum and, when present, the stratum lucidum. When this matrix melts (i.e., loses its crystalline structure), the SC becomes more permeable to molecules on the surface of the skin, allowing some molecules to diffuse inward. When the temperature of the layer returns to the normal range (i.e., 29-37° C.), the intercellular matrix recrystallizes, the SC becomes more impermeable, and any molecules which had diffused below the SC can remain there, further diffuse into surrounding tissues, or enter the systemic circulation. Thus, as used herein, the increased permeability is “reversible” because the lipid intercellular matrix recrystallizes. In different embodiments, the increase in permeability is reversed within 1 second to 2 hours after the EMR-treatment is discontinued. Thus, in some embodiments, the increase in permeability is reversed within 15 minutes, 30 minutes, 1 hour or 2 hours after the EMR-treatment is discontinued.

In these embodiments, the thermal islets define permeation pathways which can extend through or mostly through the stratum corneum and stratum lucidum layers so that a compound, for example, a cosmetic or therapeutic agent applied to the exterior surface of the skin is able to efficiently penetrate the stratum corneum/stratum lucidum. This penetration can be superficial and remain just below or within the stratum corneum, or can be deeper into the interior layers of the epidermis or dermis and, possibly, into the blood stream via the vascularization in the dermis. This enables the percutaneous delivery of cosmetic or therapeutic agents locally to the epidermis and dermis. To the extent the compound diffuses away from the site of treatment, the local delivery of the compound can be greater (e.g., delivery to a joint region). Moreover, to the extent that the compound reaches the vasculature of the dermis, delivery can be systemic.

In some embodiments, the compound is a therapeutic agent. Examples of therapeutic agents include, without limitation, a hormone, a steroid, a non-steroidal anti-inflammatory drug, an anti-neoplastic agent, an antihistamine and an anesthetic agent. Specific examples include, without limitation, hormones such as insulin and estrogen, steroids such as prednisolone and loteprednol, non-steroidal anti-inflammatory drugs such as ketorolac and diclofenac, anti-neoplastic agents such as methotrexate, and antihistamines such as histamine H1 antagonists, chlorpheniramine, pyrilamine, mepyramine, emedastine, levocabastine and lidocaine.

In other embodiments, the compound is a cosmetic agent. Examples of cosmetic agents include, without limitation, pigments (including both naturally occurring and synthetic chromophores, dyes, colorants or inks) reflective agents (including light-scattering compounds), and photoprotectants (including sunscreens). Such cosmetic agents can be used to add coloration to the skin, or to mask existing coloration (e.g., birthmarks, pigmented lesions, tattoos) by adding differently colored pigments or reflective agents. The invention provides improved methods of applying cosmetic agents because (a) the agents are contained within the stratum corneum and will not be smeared, or rubbed or washed off, and (b) the agents will remain within the stratum corneum until the cells of that layer are replaced through the normal process of outgrowth from the stratum basale (e.g., approximately 21-28 days). Thus, a single application of a cosmetic agent can last for several weeks, which can be advantageous relative to cosmetics which must be applied daily. Conversely, the application of the cosmetic agent is limited to several weeks, which can be advantageous relative to tattoos which are usually permanent unless removed by photobleaching or tissue ablation. In one embodiment, pigments for a desired temporary tattoo can be applied to the skin (e.g., by a film, brush, printing), the stratum corneum can be EMR-treated to increase permeability, and the pigments can diffuse into the skin to create the temporary tattoo. In other embodiments, an artificial tan can be created by delivering a colorant or, conversely, a tan can be prevented by delivering a sunscreen into the skin.

The increased permeability of the stratum corneum can be made painless or less painful for a subject by using lattices of thermal islets (or damage islets) rather than a continuous area of heating. Because the entire area and thickness of the skin is not heated, a 40-43° C. isotherm can be terminated near the epidermis/dermis boundary instead of deeper in the dermis. Therefore, nerve endings found in papillary dermis are not exposed to the 40-43° C. temperatures associated with a pain response. As a result, the enhanced permeability paths defined by the thermal islets can be created without pain even though the SC has been exposed to temperatures significantly higher than 40-43° C.

A significant (orders of magnitude) increase in permeability of the stratum corneum occurs when the temperature of the extracellular lipids of the SC is raised to the transition temperature, Tₘ, at which the lipid state changes from the mesomorphic (liquid crystal) state to the liquid state (Tₘ=64° C. for rat SC, see Ogiso et al. (1996), Biochim. Biophys. Acta 1301(1-2):97-104). Simple estimates of the required heat flux to achieve this temperature, and thereby reversibly melt the lipid layers of the stratum corneum, can be made as follows.

For example, the thickness of the SC can be chosen to be d=15 μm, such as can be found on the volar forearm, for the purposes of this calculation. The stratum corneum (SC) is known to be composed of a mixture of water, lipids and proteins with the following approximate weights: W1=20% water, W2=50% lipids, and W3=30% protein. The lipids of the SC are composed of the following: ceramides (50%), cholesterol (28%), free fatty acids (17%), and cholesterol sulfate (5%). The thermal parameters of the SC are determined to be the weighted sum of the corresponding parameters of the constituents with the appropriate weight factors W1, W2, and W3:

| | | | | Thermal |
|---|---|---|---|---|
| | Weight | | Specific heat, | conductivity, |
| Constituent | factor | Density, g/cm3 | J/(g K) | W/(cm K) |
| Water | 0.2 | 1 | 4.18 | 0.0058 |
| Protein | 0.3 | 1.3 | 1.55 | 0.00027 |
| Lipids | 0.5 | 0.31 (fat) | 0.975 | 0.0022 |
| Whole SC | 1 | ρ = 0.745 | c = 1.788 | κ = 2.341E−3 |
| (Avg) | | | | |

A typical initial SC temperature is T₀=30 C. The latent heat of fusion, λ, (for melting) for the SC lipids is assumed to be similar in value to that known for the lipid DPPC (dipalmitoylphosphatidylcholine). This parameter is λ=14500 J/mol=2 J/gm, where the molecular weight is 734 gm/mole. Assuming the adiabatic mode (neglect heat loss) and temperature equilibration among the constituents, the threshold fluence for melting the lipid, F, may be evaluated as follows:
Fₘ=[(Tₘ−T₀)·c+λ]·ρ·d

Using the estimates of the parameters above, the value for the required fluence to melt the lipids of the SC is Fₘ=0.07 J/cm². This fluence may be achieved in a variety of ways as discussed herein. For example, EMR may be absorbed directly and converted to heat by one or more of the constituents acting as endogenous chromophores of the SC, or EMR may be absorbed by exogenous chromophores on the skin surface (e.g., carbon dots). Note that the relative contribution of energy to actually melt the lipids is small (˜3%) and that most of the energy is needed to bring the SC from the ambient temperature, T₀, to the melting point, Tₘ.

$$\frac{λ}{[ ( Tm - T0 ) \cdot c ]} = 0.033 ,$$

The thermal relaxation time, TRT, of the SC is estimated as follows:

$$\begin{matrix}κ := 2.341 \times {10}^{- 3} \cdot \frac{watt}{cm \cdot K} {ms}_{} : = {10}^{- 3} \cdot s \\ TRT := \frac{{d}^{2} \cdot ρ \cdot c}{2 \cdot κ} TRT = 0.64 ms\end{matrix}$$

As an example, a heat flux of ˜1 kWcm⁻² for 70 μs will satisfy this condition. Note that if the melting point temperature needs to be maintained for a time exceeding the TRT, then the required heat flux must balance the heat loss once the required temperature is reached.

The size of the enhanced permeability paths can range from the diameter of an intercellular lipid space (e.g., 1 μm) or the thickness of a horny cell (e.g., 0.5 μm) at one extreme, to about the SC thickness (e.g., 10-500 μm). Typically, however, the enhanced permeability paths are about 20 μm to 1 mm in diameter and less than 50 μm in depth to avoid damage to the viable epidermal layers, as well as to reduce or eliminate pain and discomfort. Nonetheless, for some embodiments, thermal islets can extend into deeper layers of the epidermis and dermis to denature them and stimulate blood microcirculation for faster drug absorption in the body. Targeting deeper tissues with higher temperatures, however, could necessitate pain control for the patient.

Generally, the spacing of thermal islets should be as dense as possible to maximize the permeability and thus delivery efficiency. However, if the paths are too dense, then the depth-temperature selectivity is impacted. For example, if the spacing were zero, then heat would only effectively diffuse downward rather than radially, making it difficult to heat the stratum corneum sufficiently to produce enhanced permeability paths while preventing injury and pain to the deeper epidermal and dermal layers. Thus, generally, the fill factor is less than 30%, but greater than 1%, although it is not excluded that higher or lower percentage fill factors can be used for this application.

2. Thermal Islets in Deep Tissues

In accordance with the present invention, and as more fully described below, thermal islets can be produced which span from a tissue surface to deeper layers of the tissue, or which are present entirely in subsurface layers (see, e.g., FIG. 2, islet 198). Such thermal islets can be used for applications such as thermally-enhanced photobiomodulation, photobiostimulation and photobiosuspension, as well as the creation of damage islets, as described below.

C. Damage Islets

In some aspects, the invention provides methods of treating tissues by creating lattices of damage islets. These methods can be used in, for example, skin rejuvenation, tattoo removal (e.g., killing cells containing ink particles, ablation of tattoo ink particles), acne treatment (e.g., damaging or destroying sebaceous glands, killing bacteria, reducing inflammation), pigmented lesion treatment, vascular lesion treatment, and nevus flammeus (“port wine stain”) removal (e.g., reducing pathological vasculature), among others. Lattices of damage islets can also be used to increase the permeability of the stratum corneum. The time for recovery or healing of such damage islets can be controlled by changing the size of the damage islets and the fill factor of the lattice.

1. Tissue Remodeling

In one embodiment, the invention provides methods of tissue remodeling based on controlled tissue damage.

One embodiment of tissue remodeling is skin “rejuvenation,” a complex process involving one or more of (a) reduction in skin dyschromia (i.e., pigment non-uniformities), (b) reduction in telangiectasia (i.e., vascular malformations), (c) improvement in skin texture (e.g., reduction of rhytides and wrinkles, skin smoothing, pore size reduction), and (d) improvement in skin tensile properties (e.g., increase in elasticity, lifting, tightening). Techniques used for skin rejuvenation can be divided into three broad classes: ablative, non-ablative and fractional (including the lattices of islets of the present invention).

In the ablative resurfacing approach, the full thickness of the epidermis and a portion of upper dermis are ablated and/or coagulated. The ablative techniques typically deliver more pronounced clinical results, but entail considerable post-operative recovery time and care, discomfort, and risk of infection. For example, laser skin resurfacing (e.g., using a CO₂ laser an with absorption coefficient of ˜900 cm⁻¹, or an Er:YAG laser with an absorption coefficient of ˜13,000 cm⁻¹) requires weeks of recovery time, followed by a period of up to several months during which the treated skin is erythematous.

In the non-ablative approach, the zone of coagulation is shifted deeper into the tissue, with the epidermis being left intact (e.g., using lasers with absorption coefficients of 5-25 cm⁻¹). The non-ablative techniques entail considerably less post-operative recovery time and care, discomfort, and risk of infection.

The fractional approach is also non-ablative but, instead of coagulating the entire treatment area or damage zone, entails partial or fractional damage of the treatment area. That is, a lattice of damage islets is created within the treatment area.

The present invention provides methods of skin rejuvenation in which thermal and damage islets can be relatively deep in the dermis and hypodermis (e.g., depths>500 μm from the skin surface). In order to prevent epidermal damage, active or passive cooling of the epidermis can be employed.

2. Lifting and Tightening Skin

The creation of lattices of damage islets can result in skin lifting or tightening as a result of (a) shrinkage of collagen fibrils subjected to elevated temperatures (immediate effect) or (b) coagulation of localized areas in the dermis and hypodermis (immediate to short-term effect).

3. Smoothing Skin Texture

The creation of lattices of damage islets can result in smoother skin texture as a result of coagulation of localized areas in the dermis and hypodermis (immediate to short-term effect). This technique also can be used for texturing tissues or organs other than the dermis/epidermis (e.g., lip augmentation).

4. Promoting Collagen Production

The creation of lattices of damage islets can result in the promotion of collagen production as a result of the healing response of tissues to thermal stress or thermal shock (medium- to long-term effect).

5. Removing Tattoos

The creation of lattices of damage islets can be used to remove tattoos by killing the cells containing the tattoo ink particles (typically cells of the upper dermis). After these cells are killed, the tattoo ink is cleared away from the tissue site by normal scavenging processes. Alternatively, or in addition, lattices of damage islets can be used to remove tattoos by selecting the wavelength(s) of the EMR treatment to cause selective absorption of the EMR energy by the tattoo ink particles. In some embodiments, the pulse width of the incident pulse is chosen to match the thermal relaxation time of the ink particles. The absorption of the EMR energy by the tattoo ink particles can cause the cells to be heated and killed; can cause the ink particles to undergo photobleaching or be broken into smaller molecules which are removed by normal processes; or can otherwise cause the ink to be destroyed.

6. Increasing Permeability of the Stratum Corneum

The creation of lattices of damage islets can be used in order to increase the permeability of the stratum corneum by heating islets of tissue to temperatures higher than 100° C. to create small holes in SC. Thus, in these embodiments, the EMR treatment coagulates, ablates, vaporizes, or otherwise damages or removes portions of the SC, including the crystalline intercellular lipid structure or cells, to form a lattice of damage islets through the SC. This method increases the permeability of the SC for a longer period of time than the thermal islet methods described above because the damaged areas or holes can remain in the SC until that layer of cells is replaced through the normal process of outgrowth from the stratum basale (e.g., approximately 21-28 days).

7. Treating Acne

The creation of lattices of damage islets can be used to treat acne by selecting the wavelength(s) of the EMR treatment to cause selective absorption of the EMR energy by sebum, or targeting the lattice to sebaceous glands, in order to selectively damage or destroy the sebaceous glands. The EMR treatment can also be targeted to bacteria within acne sores.

8. Treating Hypertrophic Scars

The creation of lattices of damage islets can be used to treat hypertrophic scars by inducing shrinkage and tightening of the scar tissue, and replacement of abnormal connective tissue with normal connective tissue.

9. Reducing Body Odor

The creation of lattices of damage islets can be used to treat body odor by selectively targeting eccrine glands, thereby reducing the production of eccrine sweat or altering its composition.

10. Removing Warts and Calluses

The creation of lattices of damage islets can be used to treat warts and calluses by selectively targeting the pathological tissue to kill cells or cause tissue peeling. The pathological tissue can be replaced with normal tissue by normal biological processes.

11. Treating Psoriasis

The creation of lattices of damage islets can be used to treat psoriasis by using EMR of appropriate wavelength to selectively target psoriasis plaques, thereby stopping or reversing plaque formation. The pathological tissue can be replaced with normal tissue by normal biological processes.

12. Improving Wound and Burn Healing

The creation of lattices of damage islets can be used to decrease the time needed for the healing of wounds or burns (including frostbite) by increasing the wound or burn margin without substantially increasing the volume.

13. Reducing Cellulite or Fat Volume

The creation of lattices of damage islets can be used to reduce cellulite by changing the mechanical stress distribution at the dermis/hypodermis border. Alternatively, or in addition, lattices of damage islets can be used to reduce fat in the hypodermis (subcutaneous tissue) by heating and damaging fatty cells inside islets.

14. Decreasing Body Hair

The creation of lattices of damage islets can be used in order to decrease the amount or presence of body hair by targeting lattices of damage islets to hair follicles in the skin. The methods can selectively target melanin or other chromophores present in hair or hair follicles, or may non-selectively target water in the hair follicle.

15. Ablation or Welding of Internal Epithelia

The creation of lattices of damage islets can be used in order to damage or destroy internal epithelia to treat conditions such a benign prostatic hyperplasia or hypertrophy, or restenosis. The methods can also be used to weld tissues together by creating damage areas at tissue interfaces.

16. Creation of Identification Patterns

The creation of lattices of damage islets can be used in order to create identification patterns in tissues which result from the ablation of tissue or other structures, or which result from the tissue healing process. For example, patterns can be created in hair shafts by “etching” the hair with a lattice of damage islets. Alternatively, dermal, epidermal or other epithelial tissues can be patterned using the healing process to create defined areas with altered appearances.

D. Photochemical Islets

In some aspects, the invention provides methods of treating tissues by creating lattices of photochemical islets. These methods can be used in, for example, activating EMR-dependent biological responses (e.g., melanin production or “tanning”) and photodynamic therapy (e.g., psoralen therapy for vitiligo or hypopigmentation). For example, vitiligo, white stretch marks (i.e., striae alba), and hypo-pigmentation can be treated by creating photochemical islets which, with or without photodynamic agents, increase the production of pigmentation in the treated areas. In particular, by targeting the stratum basale, proliferation and differentiation of melanocytes can be promoted.

Products and Methods for Producing Lattices of EMR-Treated Islets

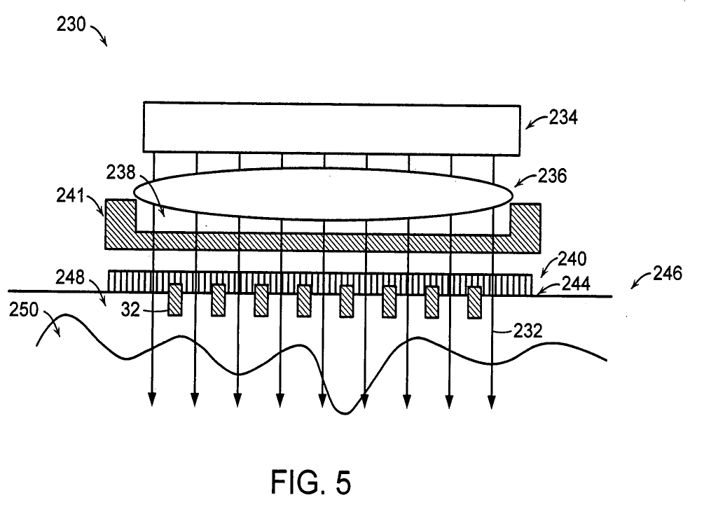

FIG. 5 shows a broad overview schematic of an apparatus 100 that can be used in one embodiment of the invention to produce islets of treatment in the patient's skin. For this apparatus 230, optical energy 232 from a suitable energy source 234 passes through optical device 236, filter 238, cooling mechanisms 240, 242, and cooling or heating plate 244, before reaching tissue 246 (i.e., the subject's skin). Each of these components is described in greater detail below. Generally, however, the EMR from the energy source 234 is focused by the optical device 236 and shaped by masks, optics, or other elements in order to create islets of treatment on the subject's skin. In some embodiments of the invention, certain of these components, such as, for example, filter 328 where a monochromatic energy source is utilized or optics 236, may not necessarily be present. In other embodiments, the apparatus may not contact the skin. In yet another embodiment, there is no cooling mechanism 4 such that there is only passive cooling between the contact plate and the skin.

A suitable optical impedance matching lotion or other suitable substance would typically be applied between plate 244 and tissue 246 to provide enhanced optical and thermal coupling. Tissue 246, as shown in FIG. 5, is divided into an upper region 248, which, for applications where radiation is applied to the skin surface, would be the epidermis and dermis, and a lower region 250, which would be a subdermal region in the previous example. Region 250, for instance, can be the hypodermis.

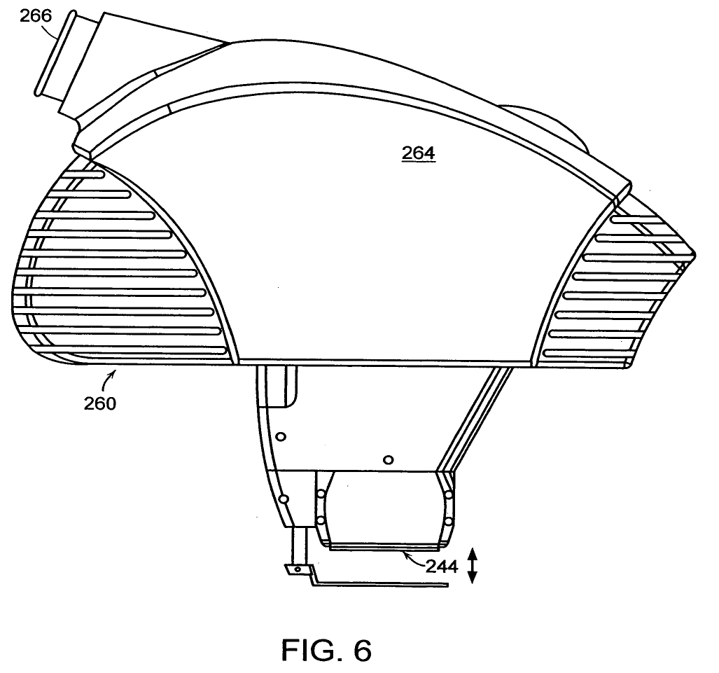

FIG. 6 shows a hand held device 260 which can contain the components of apparatus 230 set forth in connection with FIG. 5. In particular, the housing 264 of hand held device 260 can contain the energy source 264, optical device 236, filter 238, and the cooling mechanism 240 and cooling plate 244 (only cooling plate 244 is shown in FIG. 6). When in use, optical energy passes through the cooling plate 244 to contact the patient's skin. In some embodiments, the housing 264 can also support a button to activate the energy source.

The hand held device 260 of FIG. 6 also includes a connection 266 for an umbilical cord or cable connection to a control or base unit (not shown) that can communicate through control signals with the hand held device 260. The control unit can include, for example, a supply of coolant for the cooling mechanism 244. In another embodiment, the control unit can include power settings and the like for the energy source (not shown in FIG. 6) within the hand held device 260. In addition, the control unit can include a microcomputer and controller to control certain features of the invention, as will be described below in greater detail. The cable connecting the control unit to the connection 266 of the hand held device 260 can include supply lines for coolant and wires for control and power of the hand held device 260. In another embodiment, the energy source may be contained in the base unit with the energy being delivered to the hand held device through the umbilical cord. For example, optical energy may be delivered through an optical fiber in the umbilical cord. In another embodiment, all components are contained in the hand held device such that there is no base unit.

FIGS. 3A and 3B show another schematic representation of a system 208 for creating islets of treatment. FIGS. 3A and 3B show a system for delivering optical radiation to a treatment volume V located at a depth d in the patient's skin and having an area A. FIGS. 3A and 3B also show treatment or target portions 214 (i.e., islets of treatment) in the patient's skin 200. A portion of a patient's skin 200 is shown, which portion includes an epidermis 202 overlying a dermis 204, the junction of the epidermis and dermis being referred to as the dermis-epidermis (DE) junction 206. The treatment volume may be at the surface of the patient's skin (i.e., d=0) such that islets of treatment are formed in the stratum corneum. In addition, the treatment volume V may be below the skin surface in one or more skin layers or the treatment volume may extend from the skin surface through one or more skin layers.

The system 208 of FIGS. 3A and 3B can be incorporated within a hand held device, such as device 260 depicted in FIG. 6. System 208 includes an energy source 210 to produce electromagnetic radiation (EMR). The output from energy source 210 is applied to an optical system 212, which is preferably in the form of a delivery head in contact with the surface of the patient's skin, as shown in FIG. 3B. The delivery head can include, for example, a contact plate or cooling element 216 that contacts the patient's skin, as is also shown in FIG. 6 (with numeral 244). The system 208 can also include detectors 216 and controllers 218. The detectors 216 can, for instance, detect contact with the skin and/or the speed of movement of the device over the patient's skin and can, for example, image the patient's skin. The controller 218 can be used, for example, to control the pulsing of an EMR source in relation to contact with the skin and/or the speed of movement of the hand piece.

Throughout this specification, the terms “head”, “hand piece” and “hand held device” may be used interchangeably.

Each of these components is discussed in greater detail below.

A. Electromagnetic Radiation Sources

The energy source 210 may be any suitable optical energy source, including coherent and non-coherent sources, able to produce optical energy at a desired wavelength or a desired wavelength band or in multiple wavelength bands. The exact energy source 210, and the exact energy chosen, may be a function of the type of treatment to be performed, the tissue to be heated, the depth within the tissue at which treatment is desired, and of the absorption of that energy in the desired area to be treated. For example, energy source 210 may be a radiant lamp, a halogen lamp, an incandescent lamp, an arc lamp, a fluorescent lamp, a light emitting diode, a laser (including diode and fiber lasers), the sun, or other suitable optical energy source. In addition, multiple energy sources may be used which are identical or different. For example, multiple laser sources may be used and they may generate optical energy having the same wavelength or different wavelengths. As another example, multiple lamp sources may be used and they may be filtered to provide the same or different wavelength band or bands. In addition, different types of sources may be included in the same device, for example, mixing both lasers and lamps.

Energy source 210 may produce electromagnetic radiation, such as near infrared or visible light radiation over a broad spectrum, over a limited spectrum, or at a single wavelength, such as would be produced by a light emitting diode or a laser. In certain cases, a narrow spectral source may be preferable, as the wavelength(s) produced by the energy source may be targeted towards a specific tissue type or may be adapted for reaching a selected depth. In other embodiments, a wide spectral source may be preferable, for example, in systems where the wavelength(s) to be applied to the tissue may change, for example, by applying different filters, depending on the application. Acoustic, RF or other EMF sources may also be employed in suitable applications.

For example, UV, violet, blue, green, yellow light or infrared radiation (e.g., about 290-600 nm, 1400-3000 nm) can be used for treatment of superficial targets, such as vascular and pigment lesions, fine wrinkles, skin texture and pores. Blue, green, yellow, red and near IR light in a range of about 450 to about 1300 nm can be used for treatment of a target at depths up to about 1 millimeter below the skin. Near infrared light in a range of about 800 to about 1400 nm, about 1500 to about 1800 nm or in a range of about 2050 nm to about 2350 nm can be used for treatment of deeper targets (e.g., up to about 3 millimeters beneath the skin surface)—(See Table 1B).

1. Coherent Light Sources.

The energy source 210 can be any variety of a coherent light source, such as a solid-state laser, dye laser, diode laser, fiber laser, or other coherent light source. For example, the energy source 210 can be a neodymium (Nd) laser, such as a Nd:YAG laser. In this exemplary embodiment, the energy source 210 includes a neodymium (Nd) laser generating radiation having a wavelength around 1064 nm. Such a laser includes a lasing medium, e.g., in this embodiment a neodymium YAG laser rod (a YAG host crystal doped with Nd⁺³ ions), and associated optics (e.g., mirrors) that are coupled to the laser rod to form an optical cavity for generating lasing radiation. In other embodiments, other laser sources, such as chromium (Cr), Ytterbium (Yt) or diode lasers, or broadband sources, e.g., lamps, can be employed for generating the treatment radiation.

Lasers and other coherent light sources can be used to cover wavelengths within the 100 to 100,000 nm range. Examples of coherent energy sources are solid state, dye, fiber, and other types of lasers. For example, a solid state laser with lamp or diode pumping can be used. The wavelength generated by such a laser can be in the range of 400-3,500 nm. This range can be extended to 100-20,000 nm by using non-linear frequency converting. Solid state lasers can provide maximum flexibility with pulse width range from femtoseconds to a continuous wave.

Another example of a coherent source is a dye laser with non-coherent or coherent pumping, which provide wavelength-tunable light emission. Dye lasers can utilize a dye dissolved either in liquid or solid matrices. Typical tunable wavelength bands cover 400-1,200 nm and a laser bandwidth of about 0.1-10 nm. Mixtures of different dyes can provide multi wavelength emission. Dye laser conversion efficiency is about 0.1-1% for non-coherent pumping and up to about 80% with coherent pumping. Laser emission could be delivered to the treatment site by an optical waveguide, or, in other embodiments, a plurality of waveguides or laser media could be pumped by a plurality of laser sources (lamps) next to the treatment site. Such dye lasers can result in energy exposure up to several hundreds of J/cm², pulse duration from picoseconds to tens of seconds, and a fill factor from about 0.1% to 90%.

Another example of a coherent source is a fiber laser. Fiber lasers are active waveguides a doped core or undoped core (Raman laser), with coherent or non-coherent pumping. Rare earth metal ions can be used as the doping material. The core and cladding materials can be quartz, glass or ceramic. The core diameter could be from microns to hundreds of microns. Pumping light could be launched into the core through the core facet or through cladding. The light conversion efficiency of such a fiber laser could be up to about 80% and the wavelength range can be from about 1,100 to 3,000 nm. A combination of different rare-earth ions, with or without additional Raman conversion, can provide simultaneous generation of different wavelengths, which could benefit treatment results. The range can be extended with the help of second harmonic generation (SHG) or optical parametric oscillator (OPO) optically connected to the fiber laser output. Fiber lasers can be combined into the bundle or can be used as a single fiber laser. The optical output can be directed to the target with the help of a variety of optical elements described below, or can be directly placed in contact with the skin with or without a protective/cooling interface window. Such fiber lasers can result in energy exposures of up to about several hundreds of J/cm² and pulse durations from about picoseconds to tens of seconds.

Diode lasers can be used for the 400-100,000 nm range. Since many photodermatology applications require a high-power light source, the configurations described below using diode laser bars can be based upon about 10-100 W, 1-cm-long, cw diode laser bar. Note that other sources (e.g., LEDs and microlasers) can be substituted in the configurations described for use with diode laser bars with suitable modifications to the optical and mechanical sub-systems.

Other types of lasers (e.g., gas, excimer, etc.) can also be used.

2. Non-Coherent Light Sources

A variety of non-coherent sources of electromagnetic radiation (e.g., arc lamps, incandescence lamps, halogen lamps, light bulbs) can be used in the invention for the energy source 210. There are several monochromatic lamps available such as, for example, hollow cathode lamps (HCL) and electrodeless discharge lamps (EDL). HCL and EDL could generate emission lines from chemical elements. For example, sodium emits bright yellow light at 550 nm. The output emission could be concentrated on the target with reflectors and concentrators. Energy exposures up to about several tens of J/cm², pulse durations from about picoseconds to tens of seconds, and fill factors of about 1% to 90% can be achieved.

Linear arc lamps use a plasma of noble gases overheated by pulsed electrical discharge as a light source. Commonly used gases are xenon, krypton and their mixtures, in different proportions. The filling pressure can be from about several torr to thousands of torr. The lamp envelope for the linear flash lamp can be made from fused silica, doped silica or glass, or sapphire. The emission bandwidth is about 180-2,500 nm for clear envelope, and could be modified with a proper choice of dopant ions inside the lamp envelope, dielectric coatings on the lamp envelope, absorptive filters, fluorescent converters, or a suitable combination of these approaches.

In some embodiments, a Xenon-filled linear flash lamp with a trapezoidal concentrator made from BK7 glass can be used. As set forth in some embodiments below, the distal end of the optical train can, for example, be an array of microprisms attached to the output face of the concentrator. The spectral range of EMR generated by such a lamp can be about 300-2000 nm, energy exposure can be up to about 1,000 J/cm2, and the pulse duration can be from about 0.1 ms to 10 s.

Incandescent lamps are one of the most common light sources and have an emission band from 300 to 4,000 nm at a filament temperature of about 2,500 C. The output emission can be concentrated on the target with reflectors and/or concentrators. Incandescent lamps can achieve energy exposures of up to about several hundreds of J/cm² and pulse durations from about seconds to tens of seconds.

Halogen tungsten lamps utilize the halogen cycle to extend the lifetime of the lamp and permit it to operate at an elevated filament temperature (up to about 3,500 C), which greatly improves optical output. The emission band of such a lamp is in the range of about 300 to 3,000 nm. The output emission can be concentrated on the target with reflectors and/or concentrators. Such lamps can achieve energy exposures of up to thousand of J/cm² and pulse durations from about 0.2 seconds to continuous emission.

Light-emitting diodes (LEDs) that emit light in the 290-2,000 nm range can be used to cover wavelengths not directly accessible by diode lasers.

Referring again to FIGS. 3A and 3B, the energy source 210 or the optical system 212 can include any suitable filter able to select, or at least partially select, certain wavelengths or wavelength bands from energy source 210. In certain types of filters, the filter may block a specific set of wavelengths. It is also possible that undesired wavelengths in the energy from energy source 210 may be wavelength shifted in ways known in the art so as to enhance the energy available in the desired wavelength bands. Thus, filter may include elements designed to absorb, reflect or alter certain wavelengths of electromagnetic radiation. For example, filter may be used to remove certain types of wavelengths that are absorbed by surrounding tissues. For instance, dermis, hypodermis and epidermis tissues are primarily composed of water, as is much of the rest of the human body. By using a filter that selectively removes wavelengths that excite water molecules, the absorption of these wavelengths by the body may be greatly reduced, which may contribute to a reduction in the amount of heat generated by light absorption in these molecules. Thus, by passing radiation through a water-based filter, those frequencies of radiation that may excite water molecules will be absorbed in the water filter, and will not be transmitted into tissue. Thus, a water-based filter may be used to decrease the amount of radiation absorbed in tissue above the treatment region and converted into heat. For other treatments, absorption of the radiation by water may be desired or required for treatment.

B. Optical System

Generally, optical system 212 of FIGS. 3A and 3B functions to receive radiation from the source 210 and to focus/concentrate such radiation to one or more beams 222 directed to a selected one or more treatment or target portions 214 of volume V, the focus being both to the depth d and spatially in the area A (see FIG. 3B). Some embodiments of the invention use such an optical system 212, and other embodiments do not use an optical system 212. In some embodiments, the optical system 212 creates one or more beams which are not focused or divergent. In embodiments with multiple sources, optical system 212 may focus/concentrate the energy from each source into one or more beams and each such beam may include only the energy from one source or a combination of energy from multiple sources.

If an optical system 212 is used, the energy of the applied light can be concentrated to deliver more energy to target portions 214. Depending on system parameters, portions 214 may have various shapes and depths as described above.

The optical system 212 as shown in FIGS. 3A and 3B may focus energy on portions 214 or a selected subset of portions 214 simultaneously. Alternatively, the optical system 212 may contain an optical or mechanical-optical scanner for moving radiation focused to depth d to successive portions 214. In another alternative embodiment, the optical system 212 may generate an output focused to depth d and may be physically moved on the skin surface over volume V, either manually or by a suitable two-dimensional or three-dimensional (including depth) positioning mechanism, to direct radiation to desired successive portions 214. For the two later embodiments, the movement may be directly from portion to portion to be focused on or the movement may be in a standard predetermined pattern, for example a grid, spiral or other pattern, with the EMR source being fired only when over a desired portion 214.

Where an acoustic, RF or other non-optical EMR source is used as energy source 210, the optical system 212 can be a suitable system for concentrating or focusing such EMR, for example a phased array, and the term “optical system” should be interpreted, where appropriate, to include such a system.

C. Cooling Elements.

As set forth above, the system 208 can also include a cooling element 215 to cool the surface of the skin 200 over treatment volume V. As shown in FIGS. 3A and 3B, a cooling element 215 can act on the optical system 212 to cool the portion of this system in contact with the patient's skin, and thus the portion of the patient's skin in contact with such element. In some embodiments of the invention intended for use on the stratum corneum, the cooling element 215 might not be used or, alternatively, might not be cooled during treatment (e.g., cooling only applied before and/or after treatment). In some embodiments, cooling can be applied fractionally on a portion of the skin surface (cooling islets), for example, between optical islets. In some embodiments, cooling of the skin is not required and a cooling element might not be present on the hand piece. In other embodiments, cooling may be applied only to the portions of tissue between the treatment islets in order to increase contrast.

The cooling element 215 can include a system for cooling the optical system (and hence the portion in contact with the skin) as well as a contact plate that touches the patient's skin when in use. The contact plate can be, for example, a flat plate, a series of conducting pipes, a sheathing blanket, or a series of channels for the passage of air, water, oil or other fluids or gases. Mixtures of these substances may also be used, such as a mixture of water and methanol. For example, in one embodiment, the cooling system can be a water-cooled contact plate. FIG. 6, for example, shows a cooling plate 244 that is in contact with the person's skin when in use. In another embodiment, the cooling mechanism may be a series of channels carrying a coolant fluid or a refrigerant fluid (for example, a cryogen), which channels are in contact with the patient's skin 200 or with a plate of the apparatus 208 that is in contact with the patient's skin. In yet another embodiment, the cooling system may comprise a water or refrigerant fluid (for example R134A) spray, a cool air spray or air flow across the surface of the patient's skin 200. In other embodiments, cooling may be accomplished through chemical reactions (for example, endothermic reactions), or through electronic cooling, such as Peltier cooling. In yet other embodiments, cooling mechanism 215 may have more than one type of coolant, or cooling mechanism 215 and/or contact plate may be absent, for example, in embodiments where the tissue is cooled passively or directly, for example, through a cryogenic or other suitable spray. Sensors or other monitoring devices may also be embedded in cooling mechanism 215 or other portions of the hand held device, for example, to monitor the temperature, or determine the degree of cooling required by the patient's skin 200, and may be manually or electronically controlled.

In certain cases, cooling mechanism 215 may be used to maintain the surface temperature of the patient's skin 200 at its normal temperature, which may be, for example, 37 or 32° C., depending on the type of tissue being heated. In other embodiments, cooling mechanism 215 may be used to decrease the temperature of the surface of the patient's skin 200 to a temperature below the normal temperature of that type of tissue. For example, cooling mechanism 215 may be able to decrease the surface temperature of tissue to, for example, a range between 25° C. and −5° C. In other embodiments, a plate can function as a heating plate in order to heat the patient's skin. Some embodiments can include a plate that can be used for cooling and heating.

A contact plate of the cooling element 215 may be made out of a suitable heat transfer material, and also, where the plate contacts the patient's skin 200, of a material having a good optical match with the tissue. Sapphire is an example of a suitable material for the contact plate. Where the contact plate has a high degree of thermal conductivity, it may allow cooling of the surface of the tissue by cooling mechanism 215. In other embodiments, contact plate may be an integral part of cooling mechanism 215, or may be absent. In some embodiments of the invention, such as shown in FIGS. 3A and 3B, energy from energy source 210 may pass through contact plate. In these configurations, contact plate may be constructed out of materials able to transmit at least a portion of energy, for example, glass, sapphire, or a clear plastic. In addition, the contact plate may be constructed in such a way as to allow only a portion of energy to pass through contact plate, for example, via a series of holes, passages, apertures in a mask, lenses, etc. within the contact plate. In other embodiments of the invention, energy may not be directed through the cooling mechanism 215.

In certain embodiments of the invention, various components of system 208 may require cooling. For example, in the embodiment shown in FIGS. 3A and 3B, energy source 210, optics 212, and filter may be cooled by a cooling mechanism (not shown). The design of cooling mechanism may be a function of the components used in the construction of the apparatus. The cooling element 215 for the patient's skin 200 and the cooling element for the components of the system 208 may be part of the same system, separate systems or one or both may be absent. Cooling mechanism for the components of the system 208 may be any suitable cooling mechanism known in the art. Cooling of the components may be accomplished through convective or conductive cooling. In some embodiments, the cooling element can prevent optics 212 from overheating due absorption of EMR.

D. Devices for Producing a Multiplicity of Treated Islets

A number of different devices and structures can be used to spatially modulate and/or concentrate EMR in order to generate islets of treatment in the skin. For example, the devices can use reflection, refraction, interference, diffraction, and deflection of incident light to create treatment islets. A number of these devices are briefly summarized below, with a more detailed explanation of the devices in the remainder of the specification, and in particular in connection with the section entitled Devices and Systems for Producing Islets of Treatment, Example 4. Methods for generating islets of treatment, and numerous other devices and methods for creating islets of treatment are set forth throughout this specification. In addition, although some devices and methods for generating islets of treatment are briefly set forth below, the invention is not limited to these particular methods and devices.

Splitting of EMR by reflection of the light can be obtained using specular or diffuse reflection of the light from surfaces with refractive indices higher than 1. Splitting of EMR by refraction can be obtained using refraction on angular or curved surfaces. Diffraction splitting is based on the fact that light can bend around edges. Deflection splitting can be achieved when light propagates inside a media with a non-even distribution of refractive indices.

1. Blocking Portions of the EMR Beam

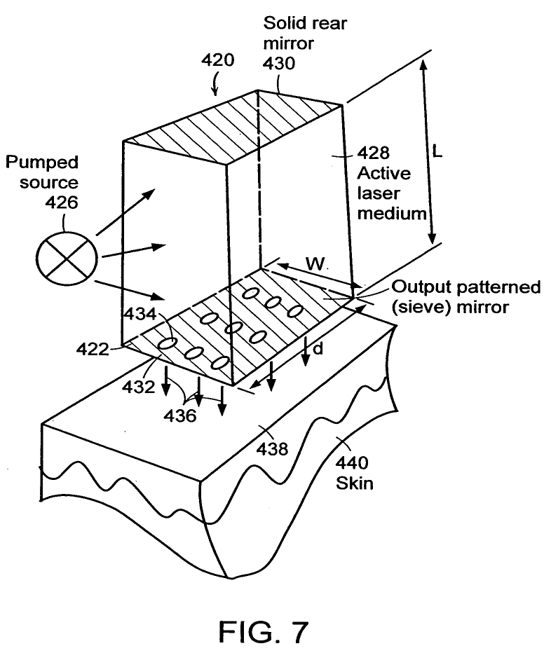

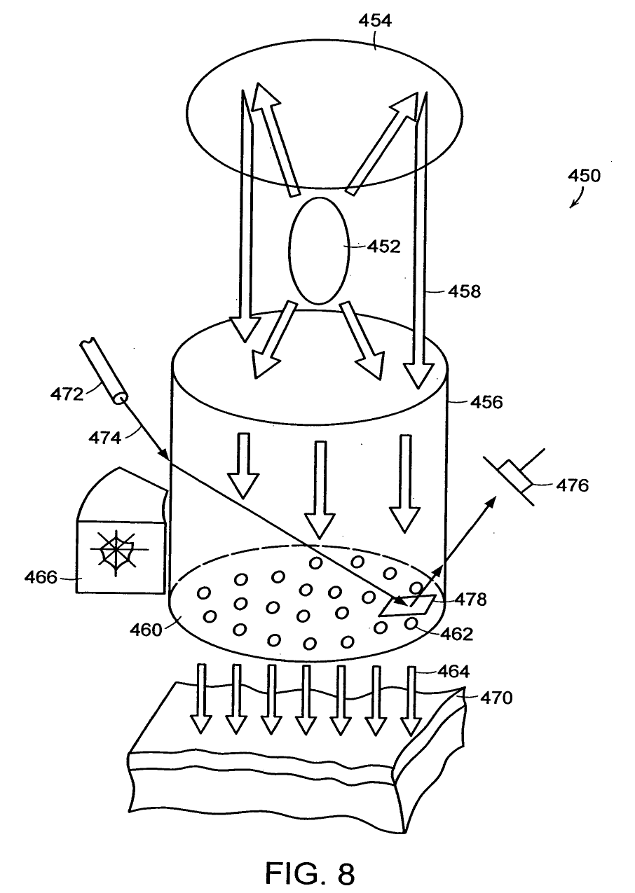

In some embodiments, a mask can be used to block portions of the EMR generated by the EMR source from reaching the tissue. The mask can contain a number of holes, lines, or slits, which function to spatially modulate the EMR to create islets of treatment. FIGS. 7 and 8 illustrate two embodiments of the invention in which the islets of treatment are formed generally through the use of a mirror containing holes or other transmissive portions. Light passes through the holes in the mirror and strikes the patient's skin, creating islets of treatment. Light reflected by the mirror stays in the optical system through a system of reflectors and may be redirected through the holes to improve efficiency. One effective mask is a contact cooling mask (i.e., it contacts the skin during treatment) with a high reflection and minimum absorption for masking light.

2. Focusing, Directing, or Concentrating the EMR Beam

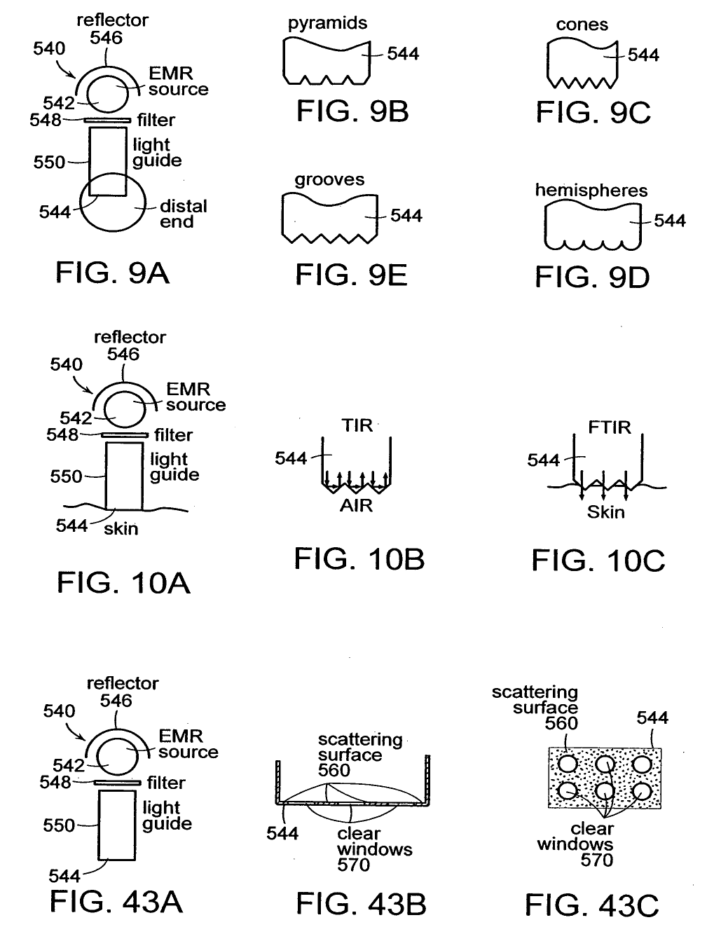

In some embodiments, spatial modulation and concentration of the EMR can be achieved by shaping an end portion of a light guide with prisms, pyramids, cones, grooves, hemispheres, or the like in order to create output light spatial modulation and concentration, and therefore to form islets of treatment in a patient's skin. For example, FIGS. 9A through 10A depict such embodiments. Numerous exemplary types of imaging optics and/or diffractive optics that can also be used in this embodiment of the invention are set forth in the section entitled Devices and Systems for Creation of Islets (Example 2) below.

In addition, in some embodiments, such as that of FIG. 10A-10C, the end of the light guide can be shaped in order to introduce light total internal reflection (TIR) when the distal end of the device is in contact with air, while allowing EMR to pass through when the distal end is in contact with a lotion or skin surface.

Alternatively, some embodiments can use spatially modulated phase arrays to introduce phase shifts between different portions of the incident beam. As a result of interference between the said portions, amplitude modulation is introduced in the output beam.

3. Arrays of EMR Sources

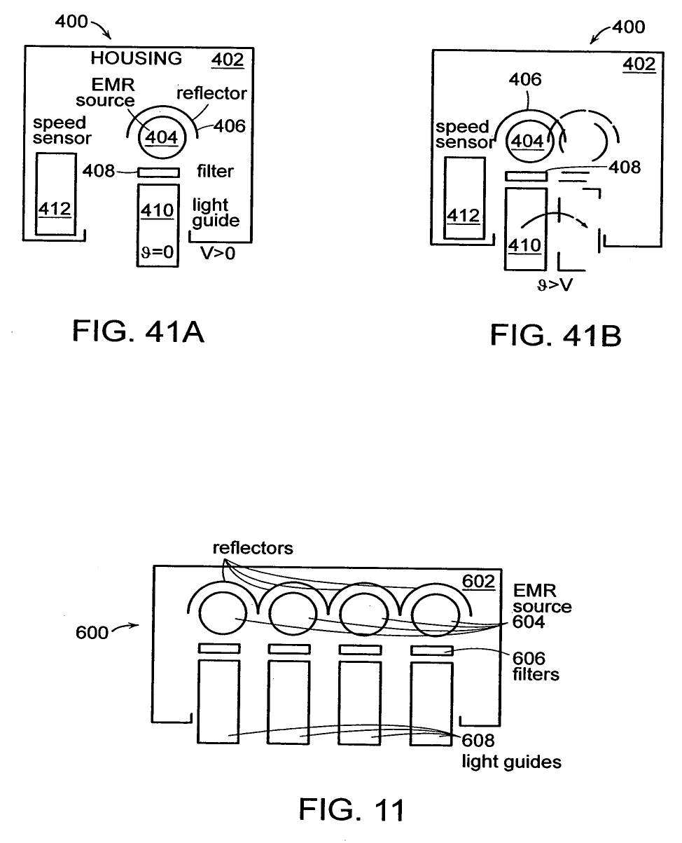

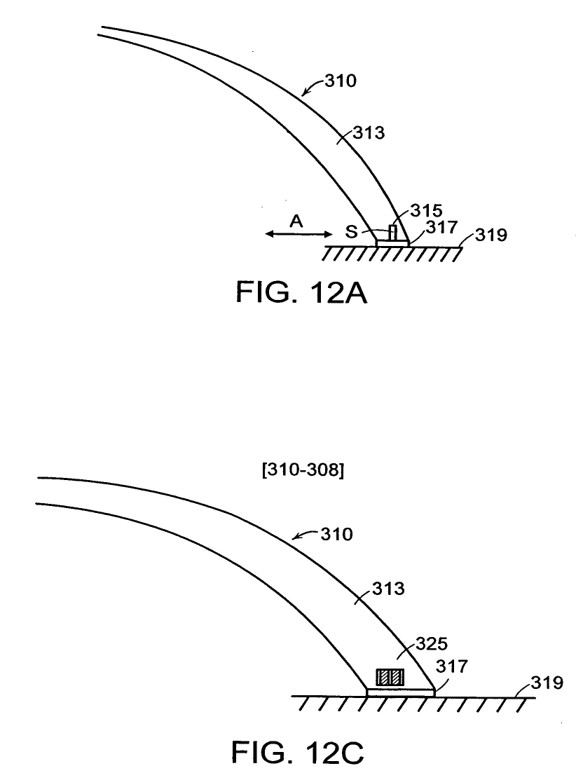

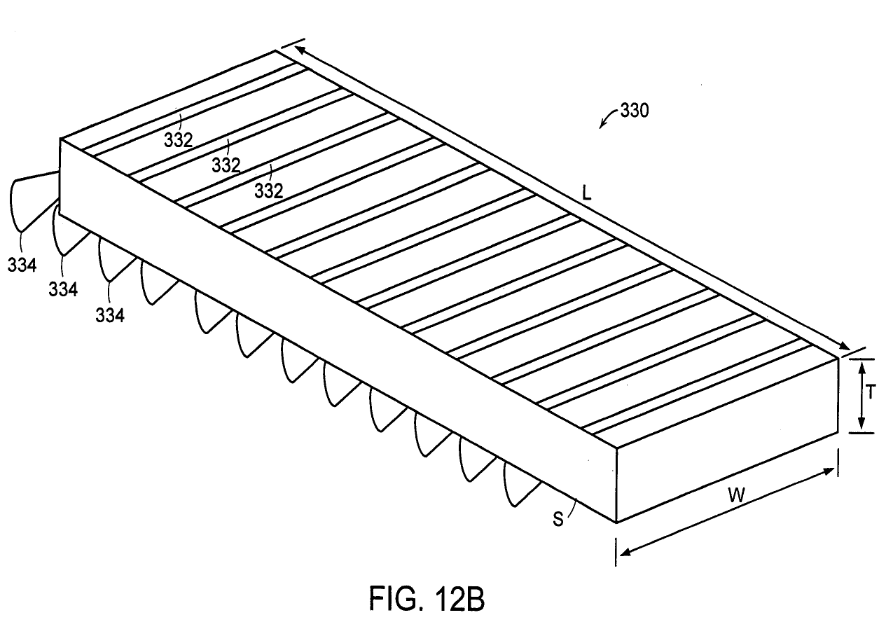

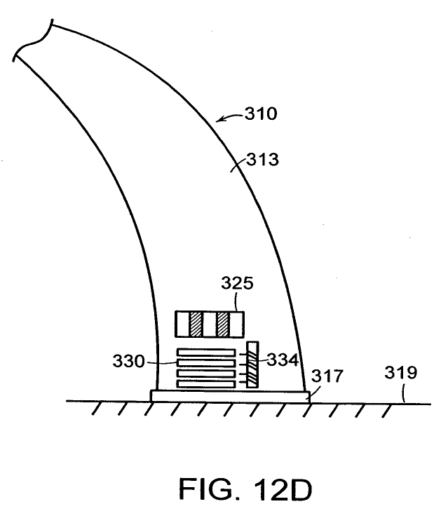

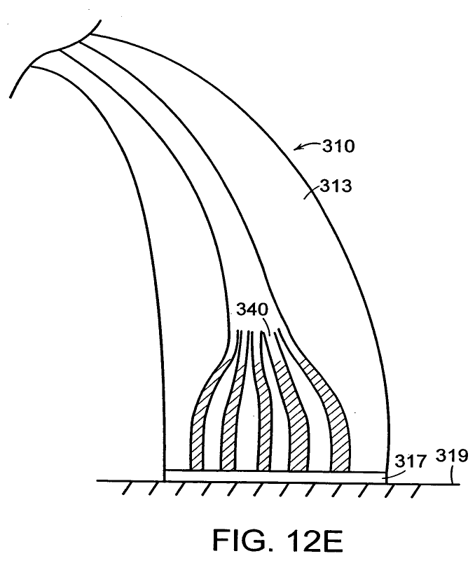

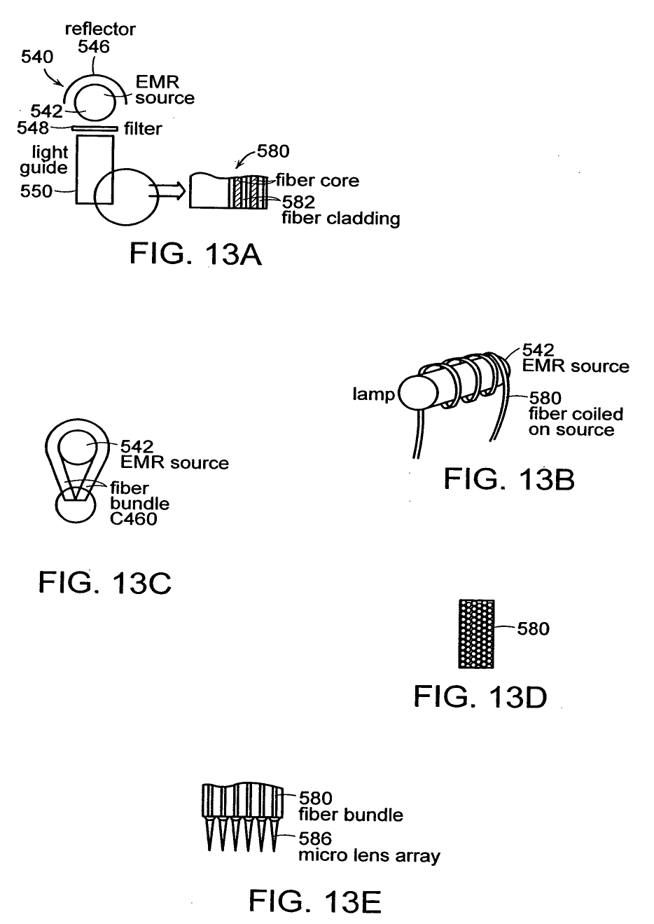

Instead of splitting the EMR into multiple beams, one can use a plurality of light sources or a single light source with a serial or parallel optical multiplexer to form islets of treatment in the patient's skin. For example, the embodiment of FIG. 11 uses a line or array of non-coherent EMR sources to create islets of treatment. Other embodiments of the invention, such as that shown in FIG. 12C, use an array of diode laser bars in order to form islets of treatment. Still other embodiments, use a bundle of optical fibers to deliver spatially modulated EMR to the patient's skin. FIGS. 12E, 13B-D, and 14A are exemplary embodiments that use a bundle of optical fibers.

4. Pulsing the EMR Source

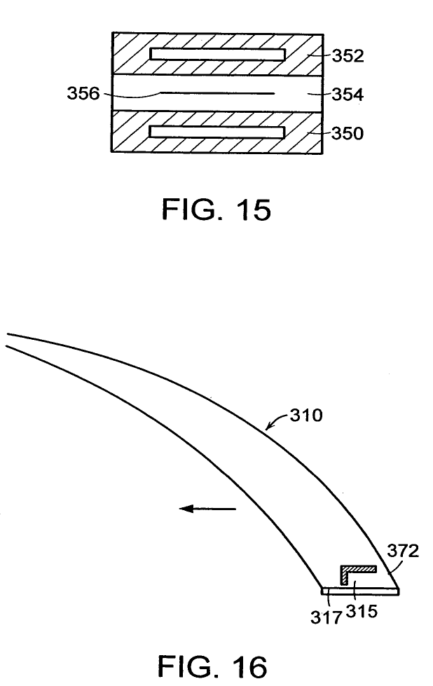

In some embodiments, the invention can include a sensor for determining the speed of movement of the hand piece across the target area of the patient's skin. The hand piece can further include circuitry in communication with the sensor for controlling the optical energy in order to create islets of treatment. The circuitry can control, for example, pulsing of the optical energy source based on the speed of movement of the head portion across the skin in order to create islets of treatment. In another embodiment, the circuitry can control movement of the energy source, a scanner or other mechanism within the apparatus based on the speed of movement of the head portion across the skin in order to expose only certain areas of the skin to the EMR energy as the head is moved over the skin in order to create islets of treatment. FIGS. 15 and 16 are exemplary embodiments according to this aspect of the invention.

5. Lattices of Exogenous Chromophores

In other embodiments, spatially selective islets of treatment can be created by applying to the skin surface a desired pattern of a topical composition containing a preferentially absorbing exogenous chromophore. The chromophore can also be introduced into the tissue with a needle, for example, a micro needle as used for tattoos. In this case, the EMR energy may illuminate the entire skin surface where such pattern of topical composition has been applied. Upon application of appropriate EMR, the chromophores can heat up, thus creating islets of treatment in the skin. Alternatively, the EMR energy may be focused on the pattern of topical composition. A variety of substances can be used as chromophores in the invention including, but not limited to, carbon, metals (Au, Ag, Fe, etc.), organic dyes (Methylene Blue, Toluidine Blue, etc.), non-organic pigments, nanoparticles (such as fullerenes), nanoparticles with a shell, carbon fibers, etc. The desired pattern can be random and need not be regular or pre-determined. It can vary as a function of the skin condition at the desired treatment area and be generated ad hoc.

In some embodiments, the invention provides a film or substrate material with a lattice of dots, lines or other shapes, either on the surface of the film or embedded within the film, in which the dots, lines or other shapes include a chromophore appropriate to the EMR source. The dots, lines or other shapes may be the same or different sizes and different shapes may be included on the film.

The dots, lines or other shapes may be formed from a material that can be glued, welded or otherwise attached to the stratum corneum to create islets, and such attachment may be sufficient to allow the film to be removed from the skin while leaving the dots, lines or other shapes on the skin. For example, the dots, lines or other shapes may be formed of an ultraviolet curing compound such that when the film is applied to the skin and ultraviolet light is applied to the film, the dots, lines or other shapes are attached to the skin and the film may be removed prior to EMR energy being applied. In other cases, the dots, lines or other shapes may be formed of a suitable phase-changing material (e.g., albumin), which can be used for welding. In other cases, the film is not removed and the EMR energy is applied through the film.

In other methods, the dots, lines or other shapes may be manually applied to the skin individually or by spraying or other techniques. In other embodiments, the hand piece may apply the shapes to the skin prior to applying the EMR energy. As one example, the shapes may be contained in a lotion, gel, powder or other topical composition that is applied to the skin manually prior to using the hand piece to apply the EMR energy. Alternatively, the lotion is dispensed by the hand piece onto the skin prior to the hand piece delivering EMR energy. As another example, a film containing the shapes may be applied to the skin manually or by the hand held device (as for example a tape dispenser).

6. Creating Thermal Lattices Using Patterned Cooling

Some embodiments can produce thermal (and damage) lattices (or treatment islets) by employing uniform EMR beams and spatially modulated cooling devices. The resulting thermal lattice in such cases will be inverted with respect to the original cooling matrix.

E. Controllers and Feedback Systems

Some embodiments of the invention can also include speed sensors, contact sensors, imaging arrays, and controllers to aid in various functions of applying EMR to the patient's skin. System 208 of FIG. 3A includes an optional detector 216, which may be, for example, a capacitive imaging array, a CCD camera, a photodetector, or other suitable detector for a selected characteristic of the patient's skin. The output from detector 216 can be applied to a controller 218, which is typically a suitably programmed microprocessor or other such circuitry, but may be special purpose hardware or a hybrid of hardware and software. Control 218 can, for example, control the turning on and turning off of the light source 210 or other mechanism for exposing the light to the skin (e.g., shutter), and control 218 may also control the power profile of the radiation. Controller 218 can also be used, for example, to control the focus depth for the optical system 212 and to control the portion or portions 214 to which radiation is focused/concentrated at any given time. Finally, controller 218 can be used to control the cooling element 215 to control both the skin temperature above the volume V and the cooling duration, both for pre-cooling and during irradiation.

F. Creation of Lattices Using Non-Optical EMR Sources

The lattices of the invention can also be produced using non-optical sources. For example, as noted above, microwave, radio frequency and low frequency or DC EMR sources can be used as energy sources to create lattices of EMR-treated islets. In addition, for treating tissue surfaces, the tissue surface can be directly contacted with heating elements in the pattern of the desired lattice.

The following examples illustrate some preferred modes of practicing the present invention, but are not intended to limit the scope of the claimed invention. Alternative materials and methods may be utilized to obtain similar results.

### EXAMPLE 1

Computational and Theoretical Models of Islets and Islet Formation

The optical, thermal and damage islets models described above were analyzed using computational models. To get a three-dimensional optical islet below the skin surface and limited from all sides, the beam can be focused into the skin. Three dimensional thermal or damage islets below the skin surface can be produced using three dimensional optical islets or using skin surface cooling in combination with optical beams with converted, diverged or collimated beams. On the other hand, two-dimensional and one-dimensional islets below or including the skin surface and three-dimensional islets including the skin surface can be obtained using a collimated beam incident normal to the skin surface. For this reason, the effects of both collimated and focused beams were considered. Furthermore, the procedures emphasized here are those where the thermal and damage islets appear due to the light absorption by the tissue water rather than by other chromophores (i.e., melanin and hemoglobin). This mechanism is characteristic for treatment in the near infrared (NIR) range. As a standard example, type II skin per Fitzpatrick's classification (Fitzpatrick (1998), Arch. Dermatol. 124:869-71) was used and the wavelength of light was assumed to be 800 nm or longer. The light pulses were generally assumed to be rectangular.

To handle the periodicity of the islets, periodic boundary conditions for light and temperature were applied at the relevant interfaces between the voxels (i.e., the periodically repeated cells that comprise the lattice, where each cell includes an islet and a portion of the space surrounding the islet). More precisely, the voxel interfaces were considered as the heat insulating surfaces showing perfect light reflection. This technique allows evaluation of solutions for light transport and heat equations within one voxel only, which can then be propagated periodically to the whole lattice.

A. Computational Model of Skin.

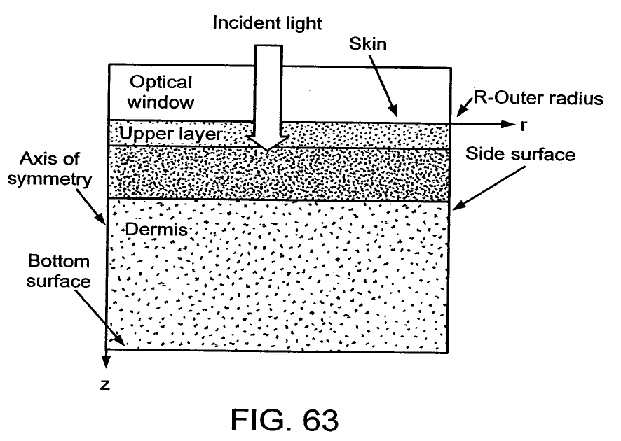

Skin was approximated by a planar four-layer structure exhibiting cylindrical symmetry as shown in FIG. 63. The particular layers included into the model were the upper layer incorporating the stratum corneum and the 3 upper layers of epidermis: the basal layer of epidermis, the reticular dermis with the upper vessel plexus, and the dermis.

In the visible and NIR spectral ranges, the absorption coefficient of each layer includes contributions from the three basic chromophores: blood, melanin, and water. The corresponding expression can be written as:
μaₖ=BₖCₖ(λ)μab(λ)+(1−Bₖ−Wₖ)·μaT(λ)+MₖμaM(λ)+WₖμaW(λ), (A1)
where k=1 . . . 4 is the layer number, Mₖ, Bₖ and Wₖ are the volume fractions of melanin, blood and water in the layer (factor Mₖ is unity for the melanin containing layers including the upper and basal layers and Mₖ is zero for the other layers), Cₖ is the correction factor, μab(λ), μaM(λ), μaW(λ) and μaT(λ) are the absorption coefficients of blood, melanin, water, and the background tissue absorption, respectively. The latter absorption coefficient is suggested to be wavelength independent and equal to 0.015 mm⁻¹. This value was obtained from the comparison of the measured and calculated spectra of the skin reflection near 800 nm, where the absorption of the main three chromophores is very small.

The correction factors are the numbers from zero through unity taking into account the fact that blood is confined to the vessels rather than being distributed homogeneously in the tissue bulk. If the vessel is thick enough, the light cannot penetrate to its inner part and, therefore, the interior of the vessel does not work as an absorber. If this is the case the correction factor is appreciably smaller than unity. Conversely, for very thin vessels the correction factor is close to unity. It follows that the correction factor depends on the mean vessel diameter and the blood absorption coefficient at the particular wavelength. To evaluate these factors, numeric data from (Verkruysse et al. (1997), Physics in Medicine and Biology 42: 51-65) were used.

Several publications address the absorption spectrum of blood (see, e.g., Roggan et al. (1999), Biomedical Optics 4: 36-46; Yaroslavsky et al. (1996), Proc. SPIE 2678: 314-24; Svaasand et al. (1995), Lasers in Medical Science 10: 55-65). The generally accepted relation is:
μab(λ)=(1−H)·μaW(λ)+H·(OS·μaHbO₂(λ)+(1−OS)·μaHb(λ)), (A2)
where H is the hematocrit (i.e. the percentage of blood volume occupied by red blood cells), OS is the oxygen saturation, μaHb(λ) and μaHbO₂(λ) are the wavelength dependent absorption coefficients of hemoglobin and oxyhemoglobin, respectively. In this invention, typical values of 0.4 for the hematocrit and 0.8 for the OS were used, the latter being the average value for the venous (0.7) and arterial (0.9) blood. The absorption spectra of hemoglobin and oxyhemoglobin, in turn, may be approximated by sums of the Gaussian bands. The intensities and widths of the bands can be found in (Douven et al. (2000), Proc SPIE 3914: 312-23).

Being the turbid medium, blood affects the scattering coefficient of the layer where it is present. The effect of blood on the total scattering coefficient is introduced by the relation (Douven et al. (2000), Proc SPIE 3914: 312-23):
μsₖ(λ)=BₖCₖμsb(λ)+(1−Bₖ)·μsTₖ(λ), (A3)
where the total scattering coefficient of blood is given by

$$\begin{matrix}μ sb ( λ ) = μ s0 \cdot H \cdot ( 1 - H ) \cdot ( 1.4 - H ) \cdot ( \frac{685 nm}{λ} ) , \\ μ s0 = 440.72 \cdot {mm}^{- 1} ,\end{matrix} \qquad ( A4 )$$

and the anisotropy factor of the blood scattering is assumed constant over the visible and NIR wavelength ranges:
gb=0.995. (A5)

The total scattering coefficient of the bloodless tissue, μsTₖ, falls with the increase of wavelength. There are several empirical relations reported in the literature to describe this dependence (Douven et al. (2000), Proc SPIE 3914: 312-23; Jacques (1996) In Advances in Optical Imaging and Photon Migration eds. Alfano et al. 2: 364-71). These relations break down above 1000 nm where the decrease of the scattering coefficient becomes very slow (Troy et al. (2001), Journal of Biomedical Optics 6: 167-176). To cover both the visible and NIR ranges, the expression for the total scattering coefficient of the bloodless tissue was rearranged in the following way:

$$μ {sT}_{k} ( λ ) = \{ \begin{matrix}μ {s0}_{k} \cdot ( \frac{577 nm}{λ} ) , & λ < 950 nm , \\ const ( λ ) , & λ \geq 950 nm ,\end{matrix} \qquad ( A6 )$$

where μs0ₖ are the scattering coefficients at the reference wavelength 577 nm listed in Table 1.

The expression for the anisotropy of scattering was constructed to include the contribution from blood in the same manner as expression (A3):

$${g}_{k} ( λ ) = \frac{{B}_{k} {C}_{k} μ sb ( λ ) gb + ( 1 - {B}_{k} ) \cdot μ {sT}_{k} ( λ ) \cdot gT ( λ )}{μ {s}_{k}} , \qquad ( A7 )$$

where gT(λ) is the anisotropy factor of the bloodless tissue. The latter factor is an increasing function of wavelength below 1000 nm and measurements using the integrated sphere technique suggest that gT(λ) does not exceed 0.9 for 1000 nm<λ<1900 nm (Troy et al. (2001), Journal of Biomedical Optics 6: 167-176). Therefore, to describe the wavelength dependence of the anisotropy factor of the bloodless tissue, the corresponding expression from (Tsai et al. (1999), Proc. SPIE 3601: 327-334) from above was limited at gT(λ)=0.9 yielding:

$$gT ( λ ) = \{ \begin{matrix}\begin{matrix}0.7645 + \\ 0.2355\end{matrix} \cdot [ 1 - exp ( - \frac{λ - 500 nm}{729.1 nm} ) ] , & λ < 1125 nm , \\ 0.9 , & λ \geq 1125 nm .\end{matrix} \qquad ( A8 )$$

Melanin is confined entirely to the epidermis with its total concentration depending on the skin type. In the context of the four-layer model used in this invention, there are two layers containing melanin: the upper and basal layers. The partitioning of melanin between the two layers depends on the skin type as well. For light skin melanin is confined mainly to the basal layer, while for dark skin the distribution of melanin in the epidermis is somewhat more homogeneous. The fraction of melanin in the basal layer was assumed to be 50% for skin types V and VI and 70% for the other skin types (Fitzpatrick (1998), Arch. Dermatol. 124: 869-71). The total amount of melanin is characterized by the optical density (OD) of the epidermis, that is, the product of the melanin absorption coefficient and the epidermal thickness. In the model described by this invention, the total OD is the sum of contributions from the upper and basal layers. Despite the OD variability due to many factors, for instance, tanning, the typical OD values listed in Table 1 were used here. These values are pertinent to the reference wavelength λₜₕ=800 nm.

Melanin OD in the infrared range can be described by the following relation:

$$OD ( λ ) = \{ \begin{matrix}OD ( 800 nm ) \cdot exp ( - \frac{λ - 800 nm}{182 nm} ) , & λ \leq {λ}_{tr} = 1000 nm , \\ OD ( 1000 nm ) \cdot {( \frac{λ}{1000 nm} )}^{- 2.14} , & λ > {λ}_{tr} .\end{matrix} \qquad ( A9 )$$

The absorption spectrum of water in the visible and near-IR may be found in the literature (Hale et al. (1973), Applied Optics 12: 555-63; Querry et al. (1978), Applied Optics 17: 3587-92). The volume fractions of water in the skin layers are listed in Table 1. The indices of refraction of the layers were assumed to be constant through the visible and NIR ranges and are listed in Table 1.

Thermal parameters of the skin layers were evaluated by applying Takata's relations (Takata et al. (1977) Laser induced thermal damage in skin. Report SAM-TR-77-38 Brooks Air Force Base (TX: US Air Force School for Aero-space Medicine)) yielding the density, the specific heat, and the thermal conductivity of a soft tissue as a function of the water content. The values obtained in this way are listed in Table 1 together with the thermal parameters of the sapphire window.

Degrees of damage were quantified by comparing the fractions of the undamaged and coagulated tissues at a particular site. Let c(t) be the fraction of the undamaged tissue at time t, so that c(0)=1. The fraction of the coagulated tissue is given by 1−c(t). The kinetic model of the tissue damage yields relation

$$\frac{ⅆ}{ⅆ t} Ω ( t ) = F ( T ( t ) ) ,$$

where Ω(t)≡ln(c(0)/c(t)), and F(T) is a function of the absolute temperature (in Kelvin) called the damage function (Pearce et al. (1995), In Optical-thermal response of laser-irradiated tissue eds. Welch et al. (NY and London: Plenum Press) pp. 561-606). The damage function used in this invention was (Pearce et al. (1995) In Optical-thermal response of laser-irradiated tissue eds. Welch et al. (NY and London: Plenum Press) pp. 561-606; Henriques (1947), Arch. Pathol. 43: 480-502; Henriques et al. (1947), Am. J. Pathol. 23: 53149; Moritz et al. (1947), Am. J. Pathol. 23:695-720; Wright (2003), J. Biomech. Eng. 125: 300-04):

$$F ( T ) = A \cdot exp ( - \frac{{E}_{a}}{R \cdot T} ) , \qquad ( A10 )$$

where R=8.31 J/(mole·K) is the universal gas constant, A is the rate constant, and Eₐ is the activation energy of the coagulation process. Given the damage function (A10), the Arrhenius damage integral was obtained:

$$Ω ( t ) = A \cdot {\int }_{0}^{t} exp ( - \frac{{E}_{a}}{R \cdot T ( {t}^{′} )} ) ⅆ {t}^{′} , \qquad ( A11 )$$

which is a measure of the damage degree (Pearce et al. (1995) In Optical-thermal response of laser-irradiated tissue eds. Welch et al. (NY and London: Plenum Press) pp. 561-606). The apparent inconvenience in using this measure is that the Arrhenius integral tends to infinity when the tissue becomes fully coagulated, i.e., c(t)→0. The more practical measure of the damage degree used here is the relative fraction change of the undamaged tissue: Ω₁=[c(0)−c(t)]/c(0)=1−exp(−Ω). The latter parameter is always positive and never exceeds unity. Clearly, Ω₁=0 indicates the absence of damage while Ω₁=1 means that the tissue is fully coagulated. It is worth noting that parameters Ω₁ and Ω are very close to each other when the damage degree is small as compared to unity. The parameter values used in the simulations here were: A=3.1·10⁹⁸ s^{—1} and Eₐ=6.28·10⁵ J/mole (Pearce et al. (1995) In Optical-thermal response of laser-irradiated tissue eds. Welch et al. (NY and London: Plenum Press) pp. 561-606).

B. Theoretical Model of Islet Lattice Relaxation.

The theory of selective photothermolysis considers the thermal relaxation time (TRT) of an individual target as the characteristic time required for an overheated target to come to the thermal equilibrium with its environment. It is suggested that the TRT is d²/(8α), d²/(16α), and d²/(24α) for the planar (one-dimensional), cylindrical (two-dimensional), and spherical (three-dimensional) targets, with d being the target width (one-dimensional) or diameter (two or three-dimensional).

This definition can be extended to an islet lattice. Significantly, if the lattice is very sparse, i.e., the fill factor is much smaller than 1, the LTRT can be almost equal to the TRT of an individual islet. It can be expected, however, that dense lattices will come to an equilibrium faster than the sparse ones, as well as that the LTRT will be determined predominantly by the dimensionality of the lattice, its fill factor, and the islet TRT.

A precise definition of LTRT was formulated as follows: let the islets be heated to temperature T₀ at time zero with the tissue temperature in between them being T_{b}<T₀. If no external action occurs, the temperature gradients in the lattice will decay in time and the lattice will approach the thermal equilibrium at stationary temperature Tₛₜ=T_{b}+(T₀−T_{b})·f. Since the stationary temperature cannot be reached for a finite time, the LTRT can be defined as the time needed for the islets to cool down to the intermediate temperature

$${T}_{1} = {T}_{st} + ( {T}_{0} - {T}_{st} ) \cdot {ⅇ}^{- 1} = {T}_{b} + ( {T}_{0} - {T}_{b} ) \cdot \frac{1 + f \cdot ( e - 1 )}{e} ,$$

with e being the natural logarithm base.

The LTRT is dependent on the lattice fill factor, f, which can be illustrated by first considering the particular case of the two-dimensional lattice. Disregarding the effect of the precise voxel and islet shapes, it can be assumed that the islet and the voxel are infinite cylinders of radii r₀ and R=r₀/\sqrt{f}, respectively. Apparently, the cylindrical pattern cannot be translated in space to form a lattice. However, it is unlikely that the transformation of the actual voxel into the cylinder of the same cross-sectional area can change the LTRT appreciably. The significance of this transformation is that it decreases the dimensionality of the problem to 1. The time-dependent heat equation within the cylindrical voxel was solved mathematically by applying a periodic (symmetry) boundary conditions on its outer surface.

Therefore, the heat equation, the initial condition, and the boundary conditions in the cylindrical frame can be written as follows:

$$\begin{matrix}ρ c \frac{\partial }{\partial t} T ( r , t ) = \frac{κ}{r} \frac{\partial }{\partial r} ( r \frac{\partial }{\partial r} T ( r , t ) ) , & ( A12 ) \\ T ( r , 0 ) = {T}_{0} \cdot \{ \begin{matrix}1 , r \leq {r}_{0} , \\ 0 , r > {r}_{0} ,\end{matrix} & ( A13 ) \\ \frac{\partial }{\partial t} T ( 0 , t ) = \frac{\partial }{\partial t} T ( R , t ) = 0 , & ( A14 )\end{matrix}$$

where ρ, c, and κ are the density, the specific heat, and the thermal conductivity of the tissue. It is suggested that T_{b}=0, which does not limit the generality of the analysis. Introducing the dimensionless time τ=t/TRT and the dimensionless coordinate ξ=r/r₀ (where TRT=d₀²/(16α)=r₀²/(4α) is the TRT of the cylindrical islet and α=κ/(ρc) is the thermal diffusivity) the following equations were obtained:

$$\begin{matrix}\frac{\partial }{\partial τ} T ( ξ , τ ) = \frac{1}{4 ξ} \frac{\partial }{\partial ξ} ( ξ \frac{\partial }{\partial ξ} T ( ξ , τ ) ) , & ( A15 ) \\ T ( ξ , 0 ) = {T}_{0} \cdot \{ \begin{matrix}1 , ξ \leq 1 , \\ 0 , ξ > 1 ,\end{matrix} & ( A16 ) \\ \frac{\partial }{\partial τ} T ( 0 , τ ) = \frac{\partial }{\partial τ} T ( \sqrt{{f}^{- 1}} , τ ) = 0. & ( A17 )\end{matrix}$$

Equations (A15)-(A17) can be solved numerically to evaluate the LTRT, that is the time when the temperature at the voxel center reduces to

$$T ( 0 , τ ) = {T}_{1} = {T}_{0} \cdot \frac{f + 1}{2} .$$

It is worth noting that set (A15)-(A17) is linear with respect to temperature and the LTRT does not depend on the initial temperature thereof. Consequently, the ratio of the LTRT to the islet TRT depends on the lattice fill factor only. Apparently, this simplification comes from the assumptions made for reducing the dimensionality of the problem.

C. Lattice Temperature Relaxation Time (LTRT).

To obtain the lattices of the thermal islets (LTI), a corresponding lattice of optical islets (LOI) has to be created first. The next step is to make the pulse width short enough to avoid overlapping of the adjacent thermal islets. It should be emphasized that LTI is a time-dependent structure and the latter requirement implies that the islets should not overlap at the time instant when the temperature reaches its maximum.

The limitation on the pulse width may be specified in the context of the theory of selective photothermolysis (Anderson et al. (1983), Science 220: 524-26; Altshuler et al. (2001), Lasers in Surgery and Medicine 29: 416-32). In its original formulation this theory deals with isolated targets inside tissue. It points out that the selective heating of a target is possible if the pulse width is smaller than some time interval characteristic for the target and referred to as the temperature relaxation time (TRT). The TRT, in essence, is the cooling time of the target, which is the time required by an instantly heated target to cool to 1/e of its initial temperature. This concept is applicable easily to the individual islets. It may be pointed out that the TRT of the planar islet (layer of the tissue, one-dimensional) is d²/(8α) with d and α being the target width and the thermal diffusivity of the tissue, respectively. For the cylindrical (two-dimensional) and spherical (three-dimensional) targets the corresponding relations read: d²/(16α) and d²/(24α) with d being the islet diameter (Altshuler et al. (2001), Lasers in Surgery and Medicine 29: 416-32). This concept was generalized to periodic lattices of the optical islets as discussed below.

It is postulated that the lattice temperature dynamics depends on the relation between the islet and voxel areas rather than by the precise islet and voxel shapes. This should be valid if the voxels are not very anisotropic, i.e., long in one direction and short in the others. The anisotropic lattices, in turn, may be considered as the lattices of smaller dimensionality. In particular, the lattice dimensionality is reduced from 2 to 1 if the voxels are very long and narrow rectangles: it is possible to switch from such rectangles to the infinitely long stripes of the same width making up a one-dimensional lattice.

Thermal dynamics of LTI depends on the method of the LOI introduction into the skin. First method is a “sequential method” or “sequential LOI”. In this case in every time instant just one (or several distant) optical islet is being created in the tissue. Laser beam scanners can be used to create sequential LOI. Second method is “parallel method” or “parallel LOI”. In this case, a multitude of optical islets are created in the tissue simultaneously during the optical pulse. Thermal interaction between islets in the sequential LOI is minimal. For parallel LOI, thermal interaction between different islets can be very significant. To evaluate the lattice thermal relaxation time (LTRT), for parallel LOI, the same reasoning used to find the TRT of an individual islet is followed. The islets are heated instantly to temperature T₀ keeping the space outside them at the constant background temperature T_{b}<T₀. By letting the islets cool through the conduction of heat to the surrounding tissue, the lattice will approach thermal equilibrium at the stationary temperature
Tₛₜ=T_{b}+(T₀−T_{b})·f, (A22)
which depends on the fill factor. The LTRT may be defined as the characteristic cooling time when the islet temperature (more precisely, the maximum temperature within the islet) reaches the intermediate value between the initial and stationary temperatures:

$${T}_{1} = {T}_{st} + ( {T}_{0} - {T}_{st} ) \cdot {ⅇ}^{- 1} = {T}_{b} + ( {T}_{0} - {T}_{b} ) \cdot \frac{1 + f \cdot ( e - 1 )}{e} . \qquad ( A23 )$$

Using this definition the LTRT of a very sparse lattice equals the TRT of an individual islet. For such a lattice each islet cools independently on the others. For denser lattices, however, the temperature profiles from different islets overlap causing the LTRT to decrease. This cooperative effect was studied by evaluating the LTRT to TRT ratio as a function of the fill factor for the particular case of the lattice of the cylindrical islets, as described herein. The LTRT decreases monotonically with the growth of the fill factor. Therefore, the denser is the islet lattice the smaller is the time while the lattice relaxes by coming down to the thermal equilibrium with the surrounding tissue. When the fill factor approaches unity, the LTRT approaches some limit close but somewhat larger than the TRT. The distinction is due to some disagreement between the definition of LTRT used here and the conventional definition of TRT. The real temperature decay is not exponential due to the heating of the surrounding tissues. Therefore, the time necessary for the target to decrease its temperature to 1/e of its initial value is always larger than TRT and this time is the actual upper limit of LTRT (the LTRT approaches this limit when the fill factor is zero).

As a rough estimate of the dependence of the LTRT to TRT ratio on the fill factor, a simple relation may be used:

$$\frac{LTRT}{TRT} \approx \frac{1}{3 \cdot f} , \qquad ( A24 )$$

providing a good fit of the numeric data for f>0.1. Actually, equation (A24) means that the LTRT is proportional to the time interval, Δ²/(2·α), while the heat front covers the distance between the islets Δ=d/\sqrt{f}. If the voxel size is very large compared to the islet diameter, the contrast of the thermal lattice may become small before the heat front covers distance Δ. Therefore, equation (A24) overestimates the LTRT appreciably if f<0.1.

D. Light Fluence Parameters for Islet Formation in a Tissue.

In order to get isolated islets, the incident fluence has to be bounded from both above and below: Fₘᵢₙ<F<Fₘₐₓ. The meaning of the latter expression is that the fluence has to be large enough to provide the desired effect within the islets but should be insufficient to cause the same effect in the whole bulk of the tissue. Practically, the right-hand-side inequality is sufficient to avoid the bulk effect in all cases while the left-hand-side warrants the formation of the islets only if the pulse width is rather short so that the relation between the delivered light energy and the attained effect is local. This means that the effect depends on the total irradiance at the same point of the tissue rather than on the average irradiance over some area. For the longer pulses, however, the dependence may become non-local due to the heat and mass transfer within the tissue (Sekins et al. (1990) In Therapeutic Heat and Cold, 4-th edition Ed. Lehmann (Baltimore: Williams & Wilkins) pp. 62-112). Therefore, the islets may not appear even if the left-hand-side inequality holds. Fₘᵢₙ can be found as a fluence needed to heat up tissue in a islet to the threshold temperature for the tissue coagulation, Tₜᵣ. If the pulse width is short enough to neglect the heat conduction, the threshold fluence for the protein coagulation is given by:
Fₘᵢₙ=ρρc(Tₜᵣ−Tᵢ)/μₐ, (A18)
where ρ is the skin density, c is its specific heat, μₐ is the skin absorption coefficient, and Tᵢ is the initial temperature. The threshold of the bulk damage Fₘₐₓ is the fluence needed to heat up tissue, both within the islets and between the islets (bulk tissue), to the threshold temperature. Because the volume of this tissue is 1/f times larger than the volume occupied by islets:
Fₘₐₓ=Fₘᵢₙ/f. (A19)

This formula is based on the assumption that the treatment is safe provided that enough intact tissue is left between the islets for assured recovery. A more conservative assumption is that, in addition to the first criterion, the treatment is safe until the temperature in the islets reaches the threshold of thermomechanical effects, Tₘₐₓ. In this case
Fₘₐₓ=Fₘᵢₙ·(Tₘₐₓ−Tᵢ)/(Tₜᵣ−Tᵢ) (A20)

The first criterion predicts a significant safety gap. For example, for f=0.25, the islets and spaces between them have equal safety margins, Fₘₐₓ/Fₘᵢₙ=4. The second criterion is more restrictive. For skin, Tₘₐₓ can be determined as the temperature of vaporization of water Tₘₐₓ=100° C. Protein coagulation temperature for ms range pulse width is Tₜᵣ=67° C. and the second criterion yields the safety margin Fₘₐₓ/Fₘᵢₙ=2.1.

A large safety margin is one of the most important features of the lattice approach. The above estimate of safety is true for periodical (regular) lattices. If lattice is irregular, islets can overlap and create large area of damage. It is the main reason why later analysis is focused on regular lattices.

Isolated islets are considered before the islet lattices. A typical method of creating a 3-dimensional (three-dimensional) optical islet is focusing light inside the skin. The optical islet of a high contrast may be obtained if the numerical aperture (NA) of the input beam is sufficiently large. However, if the NA is too large one may expect trapping and waveguide propagation of light in epidermis, which has a higher index of refraction than the underlying dermis.

E. Wavelength Dependency of Threshold Fluences.

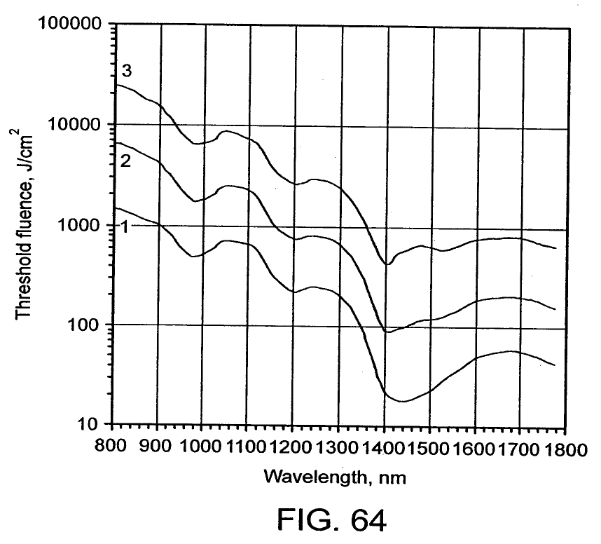

The threshold fluences for the islet treatments Fₘᵢₙ are always wavelength dependent. The particular dependence of this kind is illustrated by FIG. 64, which shows the spatially confined thermal damage of the type II skin caused by the pulses of the collimated light of diameter 0.1 mm striking the skin surface through sapphire. The pulse width was assumed to be short enough to neglect the leakage of the heat energy out of the treatment site during the pulse (the so-called adiabatic mode). If the islet is the cylinder of diameter d=0.1 mm, the temperature relaxation time is TRT=d²/(16α)≈10 ms, where α≈10⁻³ cm² s⁻¹ is the thermal diffusivity. The threshold light fluence was evaluated incident on the skin, which heated the tissue by 30° C. at the characteristic depth of 0.25 mm (curve 1), 0.5 mm (curve 2) and 0.75 mm (curve 3), respectively, and coagulated tissue up to this depth. The regions of low threshold fluence in FIG. 64 correspond to the absorption peaks of the tissue water.

The region of the low threshold fluence near 970 nm coincides with the weak water absorption peak. However, other minima are shifted from the water absorption peaks and this shift is an increasing function of the depth of damage. The reason for this is that the low threshold fluence is always a compromise between the strong absorption and low attenuation of light in the skin. Minimum threshold of damage for 0.25 mm, 0.5 mm and 0.75 mm depth was observed for 1450 nm, 1410 nm, and 1405 nm, respectively. As can be seen in FIG. 64, the threshold fluence depends on depth of tissue damage. A behavior of threshold of damage spectrum Fₜₕ(λ) is similar for all depths with an exception of the 1400-1600 nm range. In this range, damage spectrum Fₜₕ(λ) has coinciding minima for 0.25 mm and 0.5 mm depths. For a deeper damage (0.75 mm), Fₜₕ(λ) has two minima (1405 nm and 1530 nm), which are optimum wavelengths for deeper vertical cylinder type damage islets, and one maximum (1480 nm).

The important feature of plots 1-3 in FIG. 64 is the steep decrease of the threshold fluence towards the long-wavelength side that should be attributed to the decrease of the tissue scattering coefficient. Actually, the bulk scattering that causes the narrow beam to diverge while propagating into the skin reduces the tissue irradiance. For wavelengths longer than, typically, 1200 nm the scattering coefficients of the skin layers become relatively small providing the opportunity to create the cylindrical damage islets of a perfect shape at rather low fluences. The other issue is the relationship between the minima on curves 1-3 and the absorption maxima of the tissue water.

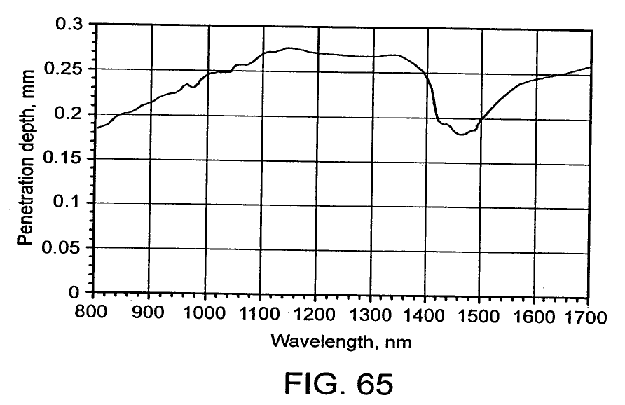

It is instructive to compare the penetration depth spectrum of FIG. 65 with the threshold fluence spectrum of FIG. 64. The comparison suggests that deeper-penetrating wavelengths may not necessarily be optimal from the viewpoint of maximizing thermal impact. Instead, the optimal wavelength for a given depth should be selected by maximizing the product of irradiance (at the depth of interest) and the absorption coefficient. For islet depths up to 0.75 mm it is reasonable to use wavelengths ranging from 1200 to 1800 nm, laying outside the strong absorption peaks of water and providing relatively low scattering of light in the tissue.

For treatment at superficial (up 0.75 mm) depth collimated beam with diameter around 0.1 can be effectively used to form LOI. To prevent stratum corneum and epidermis from damage, wavelengths with high absorption by water (around 1.45, 1.9 μm) can be used to take advantages of the low water content in stratum corneum and epidermis vs. dermis. Additionally, selective cooling of stratum corneum and epidermis can be employed. For deeper targets in the dermis and hypodermis, large sizes of optical islets have to be used.

F. Formation of Optical Islets Using the Focusing Method.

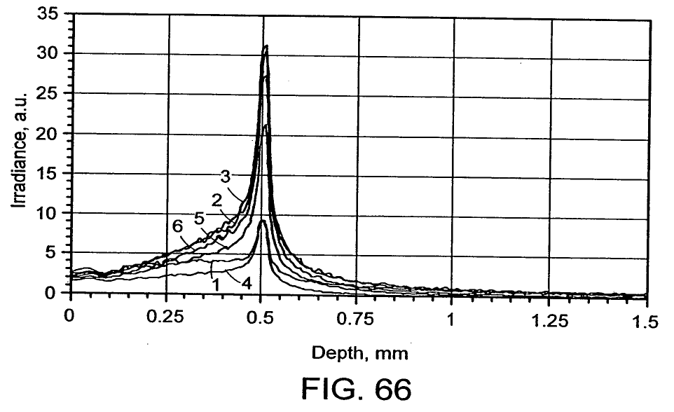

FIG. 66 illustrates the formation of the three-dimensional optical islets by the focusing method. It shows the calculated distribution of the skin irradiance on the axis of the uniform beam focused inside the type II skin (Fitzpatrick (1998), Arch. Dermatol. 124: 869-71) to the depth of 0.5 mm. The beam diameter is 1 mm so that its numerical aperture is 1. The skin irradiance was normalized to the input light fluence at the skin/sapphire boundary. Curves 1 through 6 were obtained for the specified wavelengths using the four-layer skin model described in this invention. Each curve demonstrates a sharp peak at the focusing depth—the so-called ballistic focus. This peak broadens due to multiple scattering of light on the microscopic skin heterogeneities like cell membranes, mitochondria, cell nuclei, etc. The ballistic focus itself is formed by a small portion of photons reaching the focusing depth without scattering. The contribution of the ballistic photons into the total energy balance is very small; however, these photons are concentrated in a tiny area around the focusing point forming the sharp peak of irradiance. The size of the latter area and, therefore, the height of the ballistic peak are determined by the aberrations of the ballistic light due to the macroscopic changes of the skin refraction index. The skin model described here uses different refraction indices for different layers and postulates the planar layer boundaries. Real layer boundaries are curved yielding larger aberrations than the plane boundaries. Therefore, this model may overestimate the height of the ballistic peak. The other issue is the size of the mesh elements used in the Monte-Carlo simulations. Actually, the Monte-Carlo routine of this invention evaluates the average irradiance within the voxel rather than the local irradiance at a certain point. The size of mesh elements used here was 10 μm in both directions. Smaller elements were not used because the light transport theory does not describe the microscopic oscillations of the irradiance and the voxel size has to be much larger than the wavelength.

The majority of incident photons undergo multiple scattering and do not contribute to the ballistic peak itself. However, light scattering is highly anisotropic in the NIR range. This means that the direction of the scattered photon is strongly correlated with its initial direction. For this reason, the irradiance distribution formed by the scattered light may be somewhat close to that formed by the ballistic light. Particularly, a high peak of scattered irradiance may appear around the focusing point being much wider than the ballistic peak and involving much more light energy. The composite (ballistic plus scattered) peak around the focusing point is called the “geometrical focus”. The magnitude of the irradiance maximum in the focus becomes small if the scattering coefficient is too large for a particular wavelength or the focusing is too deep.

G. Relationship Between Irradiance and Focus Depth.

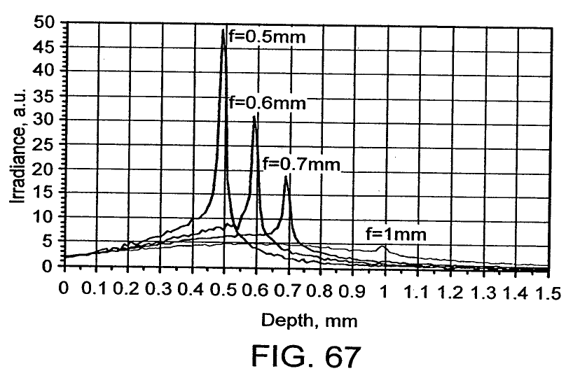

The maximum of irradiance around the focusing point decreases gradually with the increase of the focusing depth. Simultaneously, a wide peak of irradiance appears above the focusing point. The latter peak may be called “diffused focus”. This is illustrated by FIG. 67 where focusing of the 1064 nm light to depths 0.5 (1), 0.6 (2), 0.7 (3), and 1 (4) mm inside the skin through sapphire is analyzed. In the latter case, the geometrical focus can hardly be recognized whereas the “diffused” one is clearly seen.

The irradiance profile inside the skin is determined by the two competing processes: the geometrical convergence and the divergence through the multiple scattering of light in the bulk tissue. The scattering coefficient decreases gradually with the increase of the wavelength.

H. Monte-Carlo Simulations of Light Transport.

The plane or cylindrical optical islets perpendicular to the skin surface may be obtained by using a narrow collimated light beam in the skin. A beam is considered collimated in the skin if it neither converges nor diverges in a non-scattering space with the refractive index matching that of skin at the depth of treatment zₒ. Minimal diameter of collimated beam can be found from the formula (Yariv (1989) Quantum Electronics (NY: John Wiley and Sons)):
dₘᵢₙ=5(z₀λ/π)^{1/2}, (A21)
where λ is the wavelength. For typical depth zₒ=1 mm and λ1500 nm, dₘᵢₙ=0.1 mm. The spot profile may be a line (stripe) for the one-dimensional islet and some limited shape like circle or square for the two-dimensional islet. For a circular optical beam (wavelength 1200 nm) of diameter 100 μm striking the skin through sapphire, the transverse intensity profile of the beam is flat at small depths and transfers to a Gaussian when moving deeper into the skin. Therefore, the optical islet is a cylinder very sharp at the top and somewhat blurred at the bottom. It will be demonstrated below that the weak irradiance outside the original cylinder may not contribute to the tissue damage provided the pulse is short enough. This opens the opportunity of creating the damage islets of a very precise cylindrical shape.

I. Effects of Beam Diameter and Wavelength on Penetration Depth.

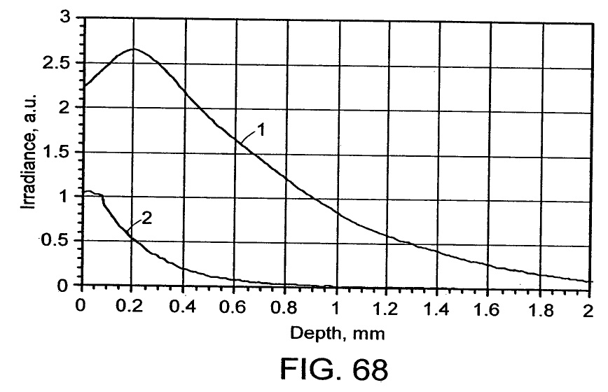

To evaluate the shape of an islet it is important to account for an effect of beam diameter on the penetration depth of light into the skin. The penetration depth is defined as the depth into the tissue where the irradiance is 1/e of the fluence incident onto the skin surface. This effect is well studied for beams wider than, typically, 1 mm (Klavuhn (2000) Illumination geometry: the importance of laser beam spatial characteristics Laser hair removal technical note No 2 (Published by Lumenis Inc)). However, if the beam is only several tens of micrometers in diameter, which is much smaller than the diffuse length of light in the skin, the propagation dynamics may be very different from that of wider beams. In particular, for such narrow beams the irradiance decreases monotonically when moving deeper into the skin along the beam axis whereas for the wider beams a subsurface irradiance maximum may occur. This is illustrated by FIG. 68, where plots 1 and 2 are for wide (diameter 10 mm) and narrow (diameter 0.1 mm) beams at wavelength 1060 nm. It should be noted herewith that the total bulk irradiance in skin is the sum of the direct and scattered components and the subsurface maximum is due to the scattered component only. When the beam diameter decreases the on-axis irradiance becomes predominantly due to the direct component and the subsurface maximum disappears.

FIG. 65 shows the wavelength dependence of the penetration depth for the uniform circular beam of incident diameter 0.1 mm. The dependence appears to be rather flat in contrast to the case of the wide beam (Jacques et al. (1995) In Optical-thermal response of laser-irradiated tissue eds. Welch et al. (NY and London: Plenum Press) pp. 561-606; Jacques (1996) In Advances in Optical Imaging and Photon Migration eds. Alfano et al. 2: 364-71; Anderson et al. (1994), Proc. SPIE MS-102: 29-35). The maximum variation of the penetration depth in the specified range is 30-35% only. The penetration depth is limited by the water absorption and the tissue scattering. Apparently, the effect of scattering is stronger for the narrow beams than for the wide ones. The tissue scattering becomes smaller with the wavelength rise while the water absorption increases. These two effects partially compensate each other and the net variations of the penetration depth are rather small.

J. Dynamics of Damage Development.

The lattices of the damage islets develop from those of the thermal islets provided certain restrictions on the pulse width and the light flux are met. The dynamics of the damage development is governed by the Arrhenius formula. The relationship between the temperature and damage islets is not straightforward. Various tissue sites may show the same peak temperature but a different damage degree, depending on the time the temperature is maintained above the activation threshold of the coagulation reaction. Moreover, if the pulse width is small the temperature islets can become very sharp at the end of the pulse. If this is the case, the steep temperature gradients may cause the islets to extend and damage the surrounding tissue after the light is off. The effect of such extension leads to onset of bulk damage when the fill factor increases beyond the safe limit.

The LOI technique has several fundamental differences and potential advantages vs. traditional treatment, which employs uniform optical beams for bulk tissue heating and damage. The following conclusions were reached from the computational and theoretical models of islets and islet formation:

(1) In addition to traditional parameters characterizing light treatment, such as the wavelength, the fluence, the pulse width and the spot size, two new important factors are introduced: the fill factor (fractional volume) and the size of islets. Furthermore, the resulting therapeutic effect can be influenced by the geometry (shape, symmetry) and dimensionality of the lattice and islets. LOI can be introduced at different depths at the tissue. For example, in the skin LOI can be localized in stratum corneum, epidermis, dermis, or hypodermis. For deep LOI, focusing technique and selective superficial cooling can be used. A suitable range of wavelengths for the LOI treatment is the near-infrared range (900-3000 nm), with water serving as the main target chromophore.

(2) The main potential advantage of the LOI approach vs. the traditional one is a significantly higher safety margin between the threshold of therapeutic effect and the threshold of unwanted side effects. The safety margin is defined as Fₘₐₓ/Fₘᵢₙ, where Fₘᵢₙ is the threshold of the desired therapeutic effect and Fₘₐₓ is the threshold of the continuous bulk damage. The theoretical upper limit for the safety margin is 1/f, where f is the fill factor of the lattice. Practically, the safety margin is determined by the expression Fₘₐₓ=Fₘᵢₙ·(Tₘₐₓ−Tᵢ)/(Tₜᵣ−Tᵢ), where Tₘₐₓ is the temperature of water vaporization, Tₜᵣ is the minimal temperature, which still provides the therapeutic effect. This margin can be up to 2 times higher than in case of traditional photothermal treatment. It should also be emphasized that the periodicity of the lattice is important for keeping the safety margin stable and for maintaining reproducibility of results.

(3) The efficacy of the lattice treatment can be increased by minimizing the size of the islets and maximizing the fill factor of the lattice. Small-size spherical or elliptical islets can be produced by using wavelengths in the 900 to 1800 nm range and focusing technique with a high numerical aperture for depth in the skin up to 0.7 mm with minimal irradiation of epidermis. The positions of the optical islets correspond to the locations of ballistic foci. For deeper focusing, the ballistic focus disappears and the maximal irradiance stabilizes at ˜0.5 mm depth (the diffuse focus).

(4) Small size column-like islets can be created in the tissue using collimated micro beams. The confocal parameter of such a beam must be longer than the depth of column in the tissue. For depths exceeding 0.5 mm, the diameter of the micro beam is generally larger than 0.1 mm. In contrast with broad beams, the depth of penetration of the micro beams is relatively insensitive to the wavelength in the range 800-1800 nm. However, the threshold fluence for tissue damage depends strongly on the wavelength. The minimal threshold fluences can be found in the range between 1380 and 1570 nm. The depth of the resulting column can be controlled by the fluence. For a superficial column with 0.25 to 0.5 mm depth, the minimal threshold fluence can be achieved in the 1400-1420 nm wavelength range and the absolute value of this fluence is between 12 and 80 J/cm². For a deeper-penetrating column of a 0.75 mm depth, the minimal threshold fluences are found at 1405 nm (400 J/cm²) and 1530 nm (570 J/cm²). In principle, a LOI can be created at a depth up to several millimeters in tissue, but in this case the size of the islets will also grow to several millimeters.

(5) The extent of the optical damage is determined by the size of the optical islets and the fluence. A damage islet is collocated with the original optical islet if the pulse width is shorter than the thermal relaxation time of the optical islet and the fluence is close to the minimal effective fluence. For higher fluences, the damage islets can grow in size even after termination of the optical pulse and, as a result, the fill factors of LTI an LDI can be higher than the fill factor of the original LOI. Islets of a lattice can be created in tissue sequentially using scanner or concurrently using lattice of optical beams. In the latter case, the optimal pulse width is shorter than the thermal relaxation time of the lattice, approximately given by LTRT=TRT/3f, where LTRT and TRT are the thermal relaxation times of the LOI and a single islet, respectively.

The concept of the lattices of optical islets can be used as a safe yet effective treatment modality in dermatology, dentistry, ophthalmology, and other biomedical applications where the target of treatment is sufficiently superficial. The same concept can be applied for other sources of energy such as microwave, radiofrequency, ultrasound, and others.

### EXAMPLE 2

Devices and Systems for Creation of Islets

One embodiment of the invention was described above in connection with FIGS. 3A and 3B. The following types of lenses and other focusing optics can be used with such an embodiment.

Lenses and Other Focusing Elements.

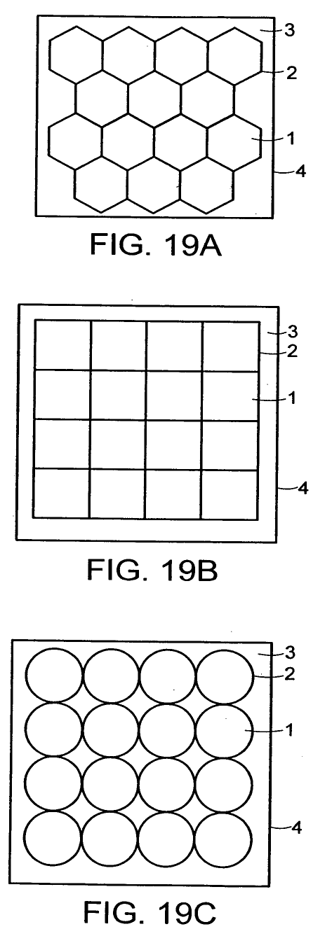

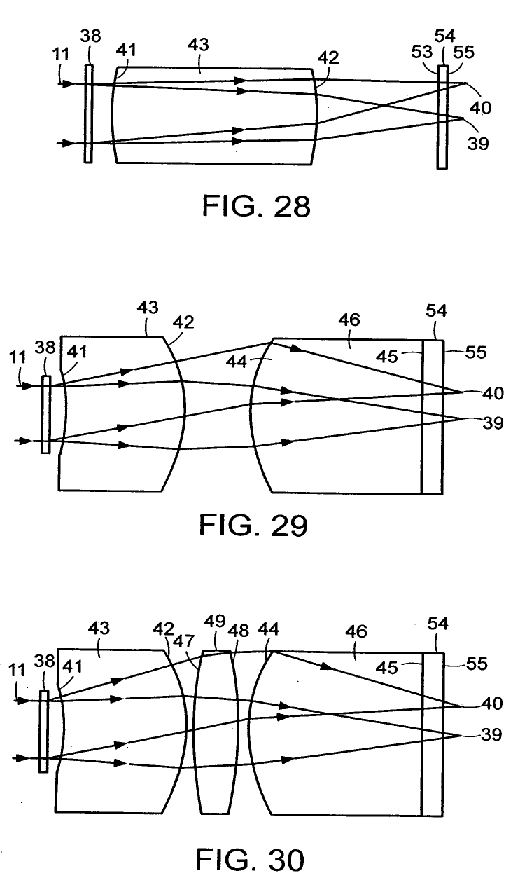

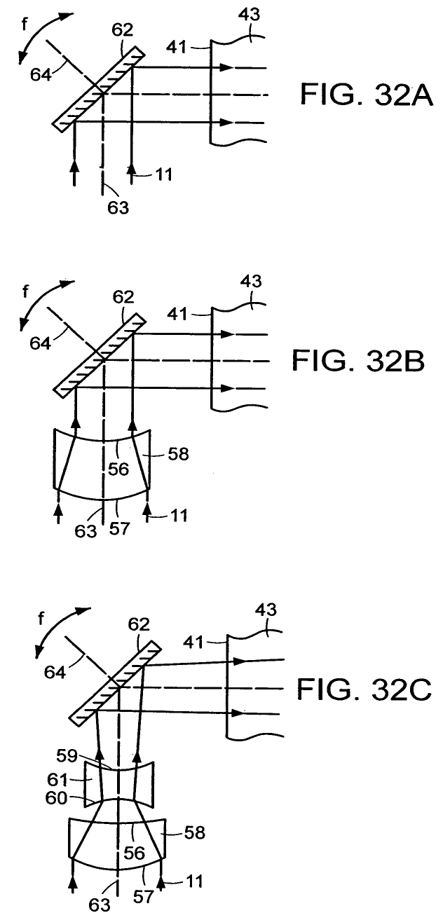

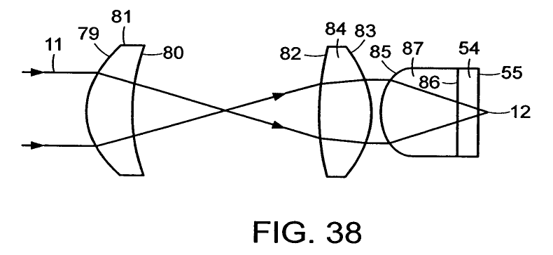

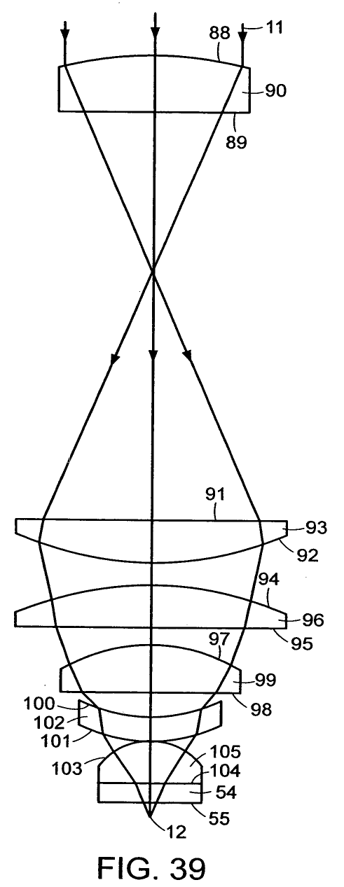

FIGS. 19A-27C illustrate various systems for delivering radiation in parallel to a plurality of target portions 214. The arrays of these figures are typically fixed focus arrays for a particular depth d. This depth may be changed either by using a different array having a different focus depth, by selectively changing the position of the array relative to the surface of the patient's skin or to target volume V or by controlling the amplitude-phase distribution of the incident radiation. FIGS. 28-31 show various optical lens arrays which may be used in conjunction with the scanning or deflector systems of FIGS. 32A-37 to move to successive one or more focused portions 214 within target volume V. Finally, FIGS. 38 and 39 show two different variable focus optical systems which may, for example, be moved mechanically or manually over the patient's skin to illuminate successive portions 214 thereon.

A. Focusing Elements

FIGS. 19A-C show a focusing element 1 on a substrate 3, the focusing element having a border which is in a hexagonal pattern (FIG. 19A), a square pattern (FIG. 19B), and a circular or elliptical pattern (FIG. 19C). Standard optical materials can be used for these elements. While the hexagonal and square patterns of FIGS. 19A and 19B can completely fill the working area of the focusing element plate 4, this is not true for the element pattern of FIG. 19C. Radiation from source 210 would typically be applied simultaneously to all of the focusing elements 1; however, the radiation could also be applied sequentially to these elements by use of a suitable scanning mechanism, or could be scanned in one direction, illuminating/irradiating for example four of the elements at a time.

B. Micro Lens Systems

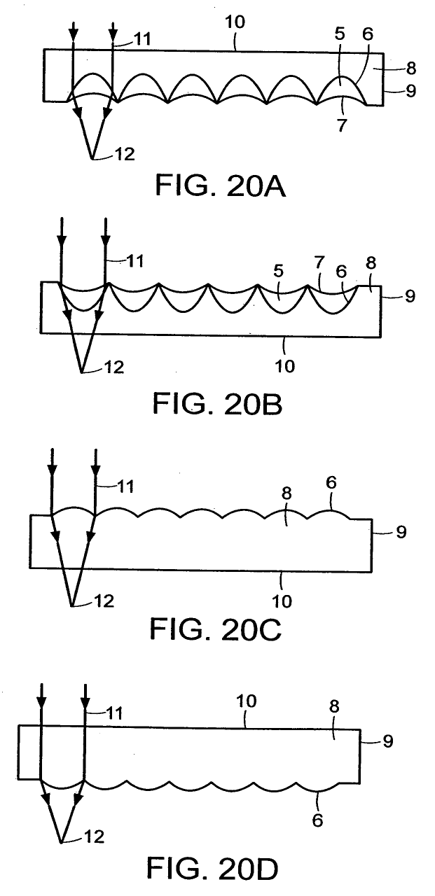

FIGS. 20A and 20B are cross-sectional views of a micro-lens system fused in a refracting material 8, for example, porous glass. The refractive index for the material of lenses 5 must be greater than the refractive index of refracting material 8. In FIG. B2, beam 11 initially passes through planar surface 10 of refracting material 8 and is then refracted both by primary surface 6 and by secondary surface 7 of each micro-lens 5, resulting in the beam being focused to a focal point 12. The process is reversed in FIG. B2A, but the result is the same. In FIGS. 20C and 20D, the incident beam 11 is refracted by a primary lens surface 6 formed of the refracting material 8. Surfaces 6 and 7 for the various arrays can be either spherical or aspherical.

C. Lenses and Lens Arrays in Immersion Materials

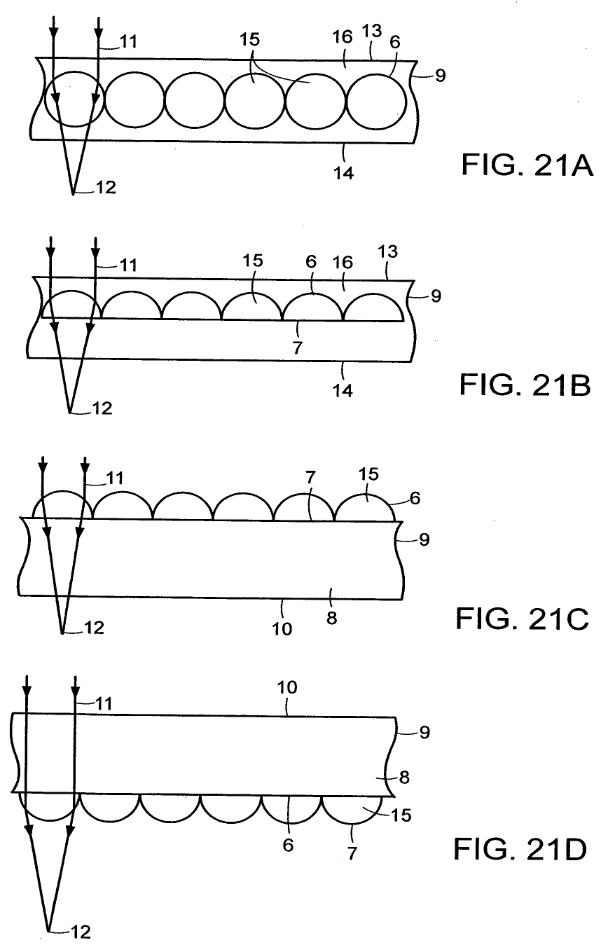

In FIGS. 21A and 21B, the lens pieces 15 are mounted to a substrate and are in an immersion material 16. The refraction index of lens pieces 15 are greater than the refraction index of immersion material 16. Immersion material 16 can be in a gas (air), liquid (water, cryogen spray) or a suitable solid gas and liquid can be used for cooling of the skin. The immersion material is generally at the primary and secondary plane surfaces, 13 and 14, respectively. The focusing depth can be adjusted by changing the refractive index of immersion material. In FIG. 21B, the primary surface 6 and secondary surface 7 of each lens piece 15 allows higher quality focusing to be achieved. For FIGS. 21C and 21D, the lens pieces 15 are fixed on a surface of a refracting material 8, the embodiment of FIG. 21D providing a deeper focus than that of FIG. 21C, or that of any of other arrays shown in FIGS. 21B-21D for a given lens 15. The lens arrays shown in FIGS. 21B-21D are preferred lens arrays for practicing the teachings of this invention.

D. Fresnel Lenses

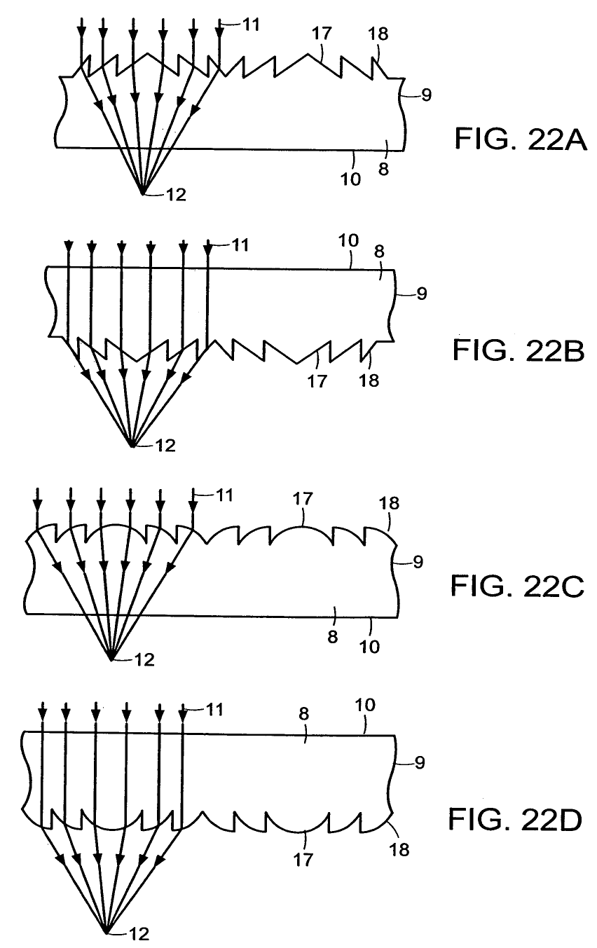

FIGS. 22A-D show Fresnel lens surfaces 17 and 18 formed on a refracting material 8. Changing the profile of Fresnel lens surface 17 and 18, the relationship between the radius of center 17 and ring 18 of the Fresnel surface, makes it possible to achieve a desired quality of focusing. The arrays of FIGS. 22C and 22D permit a higher quality focusing to be achieved and are other preferred arrays. Surfaces 17 and 18 can be either spherical or aspherical.

E. Holographic Lenses and Spatially Modulated Phase Arrays

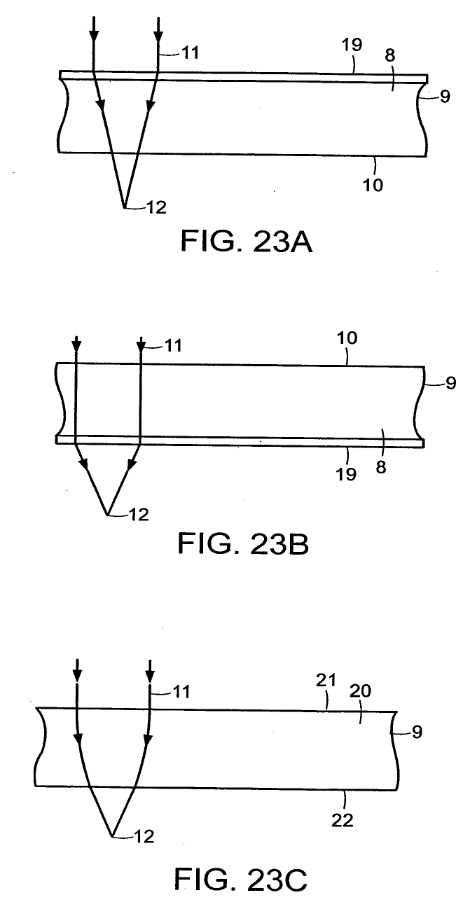

In FIGS. 23A and 23B, the focusing of an incident beam 11 is achieved by forming a holographic lens 19 on a surface of refracting material 8. Holographic lenses 19 may be formed on either of the surfaces of refracting material 8 as shown in FIGS. 23A and 23B or on both surfaces. FIG. 23C shows that the holographic material 20 substituted for the refracting material 8 of FIGS. 23A and 23B. The holographic lens is formed in the volume of material 20.

Techniques other than holography can be used to induce phase variations into different portions of the incident beam and, thus, provide amplitude modulation of the output beams.

F. Gradient Lenses

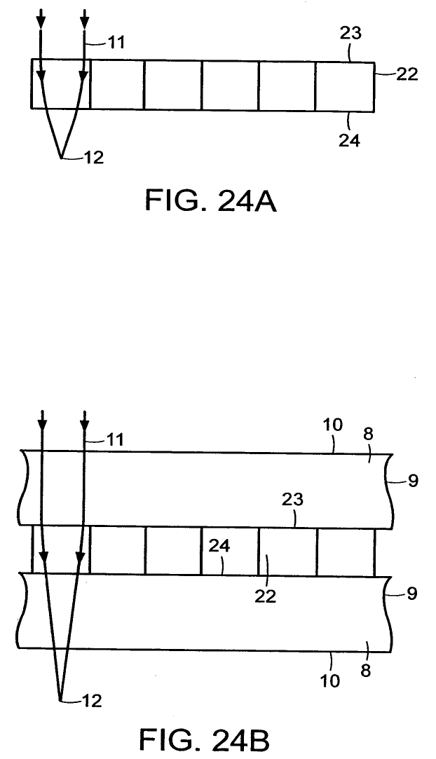

In FIGS. 24A and 24B, the focusing elements are formed by gradient lenses 22 having primary plane surfaces 23 and secondary plane surfaces 24. As shown in FIG. 24B, such gradient lenses may be sandwiched between a pair of refracting material plates 8 which provide support, protection and possibly cooling for the lenses.

G. Cylindrical Lenses

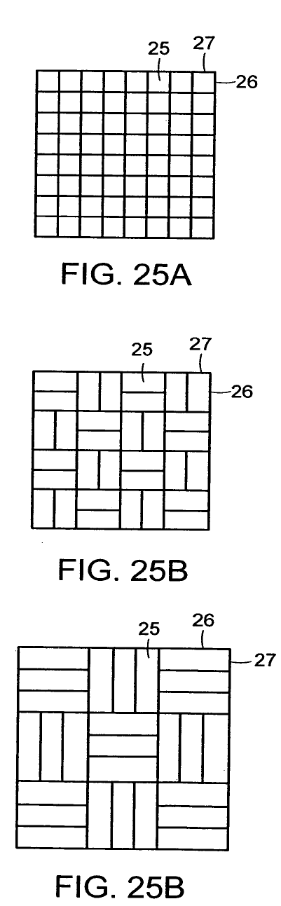

FIGS. 25A, 25B and 25C illustrate various matrix arrays of cylindrical lenses 25. The relation of the lengths 26 and diameters 27 of the cylindrical lenses 25 can vary as shown in the figures. The cylindrical lens 25 of FIGS. 25B and 25C provide a line focus rather than a spot or circle focus as for the arrays previously shown.

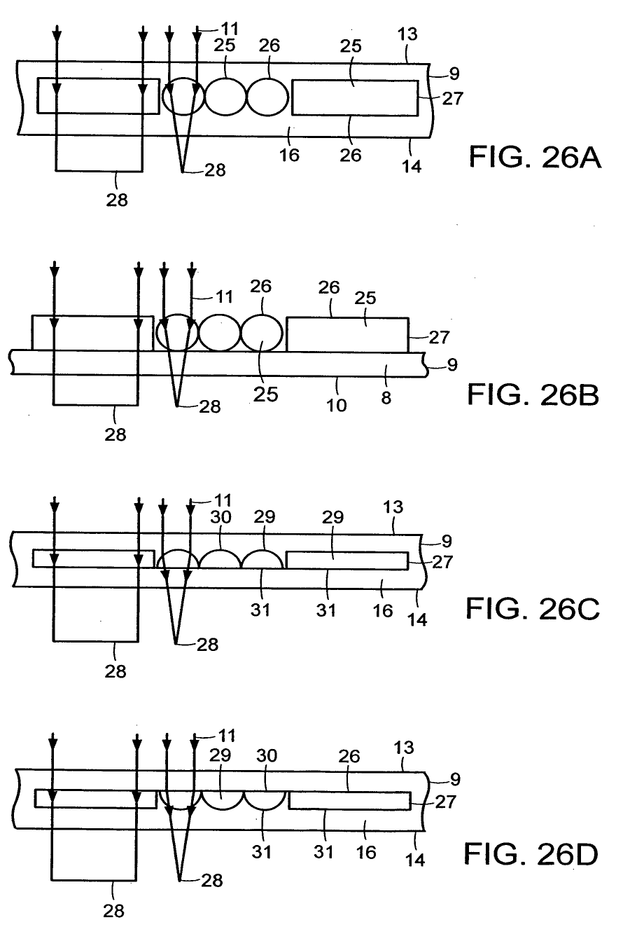

FIGS. 26A-26D are cross-sectional views of one layer of a matrix cylindrical lens system. The incident beam 11 is refracted by cylindrical lenses 25 (FIGS. 26A and 26B) or half cylinder lenses 29 (FIGS. 26C and 26D) and focus to a line focus 28. In FIGS. 26C and 26D, the cylindrical lenses 29 are in the immersion material 16. Primary working optical surface 30 and secondary optical working surface 31, which may be spherical or aspherical, allowing high quality focusing to be achieved. As shown in FIGS. 25A-26D the line focuses for adjacent lenses may be oriented in different directions, the orientations being at right angles to each other for certain of the lenses in these figures.

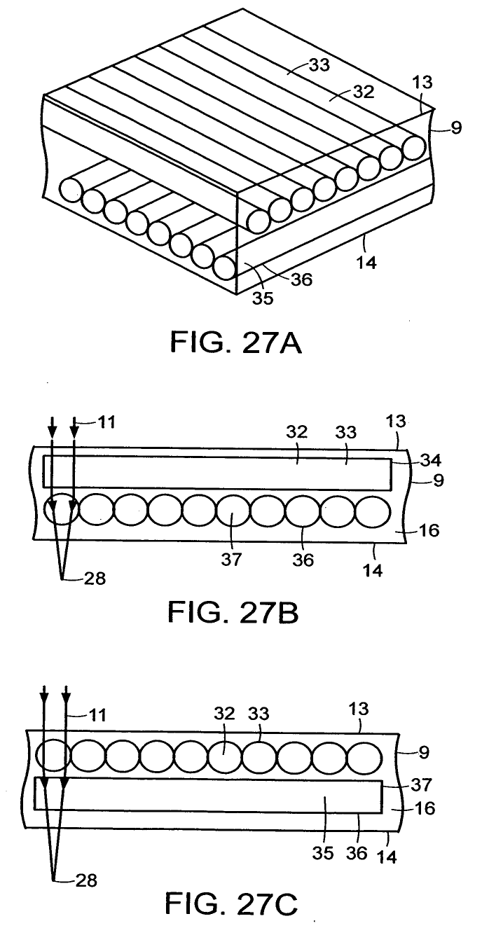

In FIGS. 27A, 27B and 27C, a matrix of focal spots is achieved by passing incident beam 11 through two layers of cylindrical lenses 32 and 35. FIGS. 27B and 27C are cross-sections looking in two orthogonal directions at the array shown in FIG. 27A. By changing the focal distance of primary layer lens 32, having a surface 33, and secondary lens 35, having a surface 36, it is possible to achieve a rectangular focal spot of a desired size. Primary layer lens 32 and secondary layer lens 35 are mounted in immersion material 16. Lenses 32 and 35 may be standard optical fibers or may be replaced by cylindrical lenses, which may be spherical or aspherical. Surfaces 34 and 37 can be of optical quality to minimize edge losses.

Described above optical system can be used with a pulse laser (0.1-100 ms) to introduce simultaneously into the skin a lattice of optical islets. For example it can be an Er:glass laser (1.56 microns wavelength) or a Nd:YAG laser (1.44 microns) with fiber delivery and imaging optics to formed uniform beam before focusing elements.

H. One, Two, and Three-Lens Objectives

FIG. 28 shows a one-lens objective 43 with a beam splitter 38. The beam 11 incident on angle beam splitter (phase mask) 38 divides and then passes through the refracting surfaces 41 and 42 of lens 43 to focus at central point 39 and off-center point 40. Surfaces 41 and 42 can be spherical and/or aspherical. Plate 54 having optical planar surfaces 53 and 55 permits a fixed distance to be achieved between optical surface 55 and focusing points 39, 40. Angle beam splitter 38 can act as an optical grating that can split beam 11 into several beams and provide several focuses.

In FIG. 29, a two lens 43,46 objective provides higher quality focusing and numerical aperture as a result of optimal positioning of optical surfaces 41, 42 and 44. All of these surfaces can be spherical or aspherical. Optical surface 45 of lens 46 can be planar to increase numerical aperture and can be in contact with plate 54. Plate 54 can also be a cooling element as previously discussed.

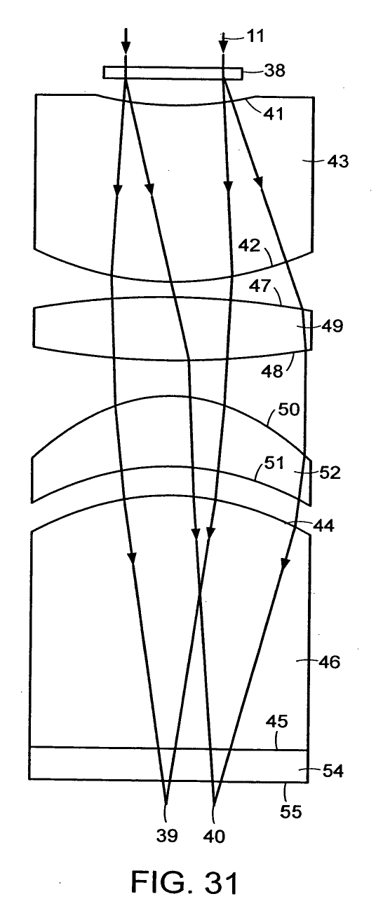

FIG. 30 differs from the previous figures in providing a three-lens objective, lenses 43, 46 and 49. FIG. 31 shows a four lens objective system, the optical surfaces 50 and 51 of lens 52 allowing an increased radius of treatment area (i.e., the distance between points 39 and 40).

I. Mirror-Containing Optical Systems

FIGS. 32A, 32B and 32C illustrate three optical systems, which may be utilized as scanning front ends to the various objectives shown in FIGS. 28-31. In these figures, the collimated initial beam 11 impinges on a scanning mirror 62 and is reflected by this mirror to surface 41 of the first lens 43 of the objective optics. Scanning mirror 62 is designed to move optical axis 63 over an angle f. Rotational displacement of a normal 64 of mirror 62 by an angle f causes the angle of beam 11 to be varied by an angle 2f. The optical position of scanning mirror 62 is in the entrance pupil of the focusing objective. To better correlate between the diameter of scanning mirror 62 and the radius of the working surface (i.e., the distance between points 39 and 40) and to increase the focusing quality, a lens 58 may be inserted before scanning mirror 62 as shown in FIG. 32B. Optical surfaces 56 and 57 of lens 58 can be spherical or aspherical. For additional aberration control, a lens 61 may be inserted between lens 58 and mirror 62, the lens 61 having optical surfaces 59 and 60.

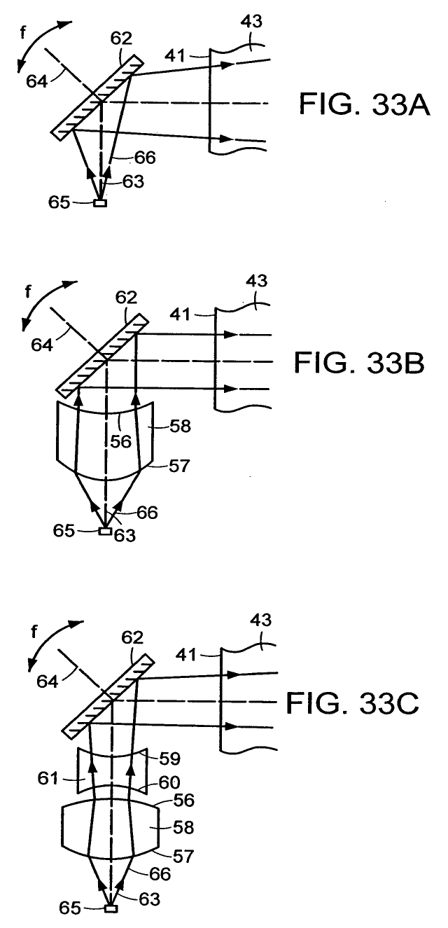

FIGS. 33A, 33B and 33C are similar to FIGS. 32A, 32B and 32C except that the light source is a point source or optical fiber 65 rather than collimated beam 11. Beam 66 from point source 65, for example the end of a fiber, is incident on scanning mirror 62 (FIG. 33A) or on surface 57 of lens 58 (FIGS. 33B and 33C).

J. Scanning Systems

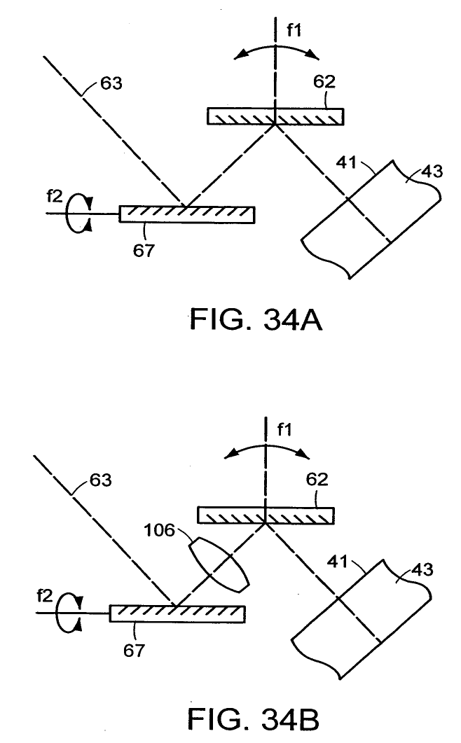

FIGS. 34A and 34B show a two mirror scanning system. In the simpler case shown in FIG. 34A, scanning mirror 67 rotates over an angle f2 and scanning mirror 62 rotates over an angle f1. Beam 63 is initially incident on mirror 67 and is reflected by mirror 67 to mirror 62, from which it is reflected to surface 41 of optical lens 43. In FIG. 34B, to increase the numerical aperture of the focusing beam, increase work area on the skin and decrease aberration between scanning mirrors 62 and 67, an objective lens 106 is inserted between the mirrors. While a simple one-lens objective 106 is shown in this figure, more complex objectives may be employed. Objective lens 106 refracts the beam from the center of scanning mirror 67 to the center of scanning mirror 62.

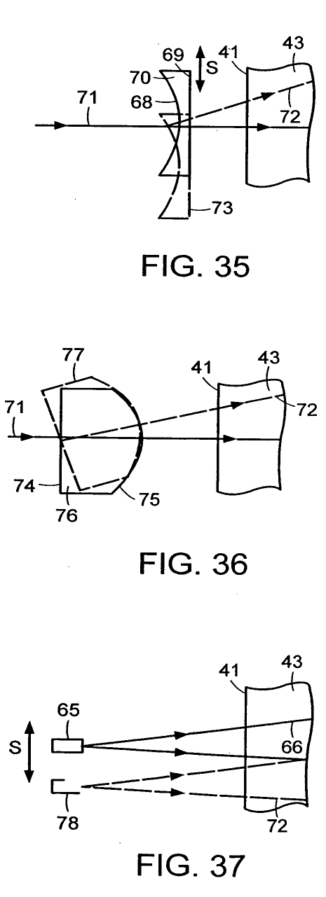

In FIG. 35, scanning is performed by scanning lens 70, which is movable in direction s. When scanning lens 70 is moved to an off center position 73, optical surface 68 refracts a ray of light along optical axis 71 to direction 72.

In FIG. 36, scanning is performed by rotating lens 76 to, for example, position 77. Surface 74 is planar and surface 75 is selected so that it does not influence the direction of refracted optical axis 72. In FIG. 37, scanning is performed by the moving of point source or optical fiber 65 in directions.

K. Zoom Lens Objectives

FIGS. 38 and 39 show zoom lens objectives to move the damage islets to different depths. In FIG. 38, a first component is made up of a single lens 81 movable along the optical axis relative to a second component, which is unmovable and consists of two lenses 84 and 87. Lens 84 is used to increase numerical aperture. To increase numerical aperture, range of back-focal distance and decrease focal spot size, optical surfaces 79, 80, 82, 83 and 85 can be aspherical. The relative position of the first and second components determines the depth of focal spot 12.

FIG. 39 shows zoom lens objectives with spherical optical surfaces. The first component is made up of a single lens 90 movable with respect to the second component along the optical axis. The second component, which is unmovable, consists of five lenses 93, 96, 99, 102, and 105. The radius of curvature of surfaces 88 and 89 are selected so as to compensate for aberrations of the unmovable second component. Again, the depth of focus may be controlled by controlling the distance between the first and second components. Either of the lens systems shown in FIGS. 38 and 39 may be mounted so as to be movable either manually or under control of control 218 to selectively focus on desired portions 214 of target volume V or to non-selectively focus on portions of the target volume.

L. Focus Depth.

While as may be seen from Table B1, depth d for volume V and the focal depth of optical system 212 are substantially the same when focusing to shallow depths, it is generally necessary in a scattering medium such as skin to focus to a greater depth, sometimes a substantially greater depth, in order to achieve a focus at a deeper depth d. The reason for this is that scattering prevents a tight focus from being achieved and results in the minimum spot size, and thus maximum energy concentration, for the focused beam being at a depth substantially above that at which the beam is focused. The focus depth can be selected to achieve a minimum spot size at the desired depth d based on the known characteristics of the skin.

M. Wavelength.

Both scattering and absorption are wavelength dependent. Therefore, while for shallow depths a fairly wide band of wavelengths can be utilized while still achieving a focused beam, the deeper the focus depth, the more scattering and absorption become factors, and the narrower the band of wavelengths available at which a reasonable focus can be achieved. Table B1 indicates preferred wavelength bands for various depths, although acceptable, but less than optimal, results may be possible outside these bands.

TABLE B1

| Depth of | | |
|---|---|---|
| damage, μm | Wavelength range, nm | NumericalAperture range |
| 0-200 | 290-10000 | <3 |
| 200-300 | 400-1880 & 2050-2350 | <2 |
| 300-500 | 600-1850 & 2150-2260 | <2 |
| 500-1000 | 600-1370 & 1600-1820 | <1.5 |
| 1000-2000 | 670-1350 & 1650-1780 | <1 |
| 2000-5000 | 800-1300 | <1 |

N. Pulse Width.

Normally the pulse width of the applied radiation should be less than the thermal relaxation time (TRT) of each of the targeted portions or optical islets 214, since a longer duration will result in heat migrating beyond the boundaries of these portions. Since the portions 214 will generally be relatively small, pulse durations will also be relatively short. However, as depth increases, and the spot sizes thus also increase, maximum pulse width or duration also increase. The pulse-widths can be longer than the thermal relaxation time of the target portion 214 if density of the targets is not too high, so that the combined heat from the target areas at any point outside these areas is well below the damage threshold for tissue at such point. Generally, thermal diffusion theory indicates that pulse width τ for a spherical islet should be τ<500 D²/24 and the pulse width for a cylindrical islet with a diameter D is τ<50 D²/16, where D is the characteristic size of the target. Further, the pulse-widths can sometimes be longer than the thermal relaxation time of the target portion 214 if density of the targets is not too high, so that the combined heat from the target areas at any point outside these areas is well below the damage threshold for tissue at such point. Also, as will be discussed later, with a suitable cooling regimen, the above limitation may not apply, and pulse durations in excess of the thermal relaxation time for a damage portion 214, sometimes substantially in excess of TRT, may be utilized.

O. Power.

The required power from the radiation source depends on the desired therapeutic effect, increasing with increasing depth and cooling and with decreasing absorption due to wavelength. The power also decreases with increasing pulse width.

P. Cooling.

Typically cooler 215 is activated before source 210 to pre-cool the patient's skin to a selected temperature below normal skin temperature, for example −5° C. to 10° C., to a depth of at least DE junction 206, and preferably to depth d to protect the entire skin region 220 above volume V. However, in accordance with the teachings of this invention, if pre-cooling extends for a period sufficient for the patient's skin to be cooled to a depth below the volume V, and in particular if cooling continues after the application of radiation begins, then heating will occur only in the radiated portions 214, each of which portions will be surrounded by cooled skin. Therefore, even if the duration of the applied radiation exceeds TRT for portions 214, heat from these portions will be contained and thermal damage will not occur beyond these portions. Further, while nerves may be stimulated in portions 214, the cooling of these nerves outside of portions 214 will, in addition to permitting tight control of damage volume, also block pain signals from being transmitted to the brain, thus permitting treatments to be effected with greater patient comfort, and in particular permitting radiation doses to be applied to effect a desired treatment which might not otherwise be possible because of the resulting pain experienced by the patient. This cooling regimen is an important feature of this invention.

Q. Numerical Aperture.

Numerical aperture is a function of the angle 9 for the focused radiation beam 222 from optical device 212. It is preferable that this number, and thus the angle 9, be as large as possible so that the energy at portions 214 in volume V where radiation is concentrated is substantially greater than that at other points in volume V (and in region 220), thereby minimizing damage to tissue in region 220, and in portions of volume V other than portions 214, while still achieving the desired therapeutic effect in the portions 214 of volume V. Higher numerical aperture of the beam increases safety of epidermis, but it is limited by scattering and absorption of higher incidence angle optical rays. As can be seen from Table B1, the possible numerical aperture decreases as the focus depth increases.

### EXAMPLE 3

Enhanced-Penetration Channels and Optical Clearance of Pig Skin In Vitro

A lattice of damage islets was created in the stratum corneum of farm pig skin using a standard flash-arc-lamp system that emits in the 650-1200 nm band (StarLux Rs™, Palomar Medical Technologies, Burlington, Mass.) and a damage islet mask consisting of carbon particles in a film which was applied to the surface of the skin. Furthermore, to determine optical clearance of treated areas of pig skin specimens, a 40% solution of glucose in water was applied to the surface of the specimen. Optical clearance refers to a change in optical properties of the tissue which makes it more transparent in the optical range by reducing light scattering. Permeation of the skin by glucose or glycerin increases the optical clearance by reducing the refractive index differences between the interstitial solution and the intercellular matrix proteins collagen and elastin.

In a first set of experiments, an approximately 4 cm² farm pig skin specimen was glued (LOCTITE 411 glue) to a rigid transparent substrate and cleaned with an alcohol wipe. The dry skin surface was divided into four 1 cm² areas. A damage islet mask was placed on the surface of the specimen and covered with a thin layer of lotion (LuxLotion™, Palomar Medical Technologies, Burlington, Mass.) to imporve optical coupling to the light source. Two of the four 1 cm² areas of the specimen received two pulses (duration 20 ms) at 36 J/cm² using the StarLux Rs hand piece. One 1 cm² area of the specimen received two pulses (duration 10 ms) at 20 J/cm². The fourth 1 cm² area of the specimen served as a non-treated control. The distances between the treated areas were approximately 1 cm. After treatment, loose carbon particles on the surface were removed, and the specimen was covered with 40% solution of glucose, and kept warm using a hair dryer. The surface of the sample was kept wet by adding fresh glucose solution.

Thin blue wires were placed under the test areas of the specimen after the treatment, and optical clearance of the tissue was assessed by observation of visual appearance of blue wires through the specimen.

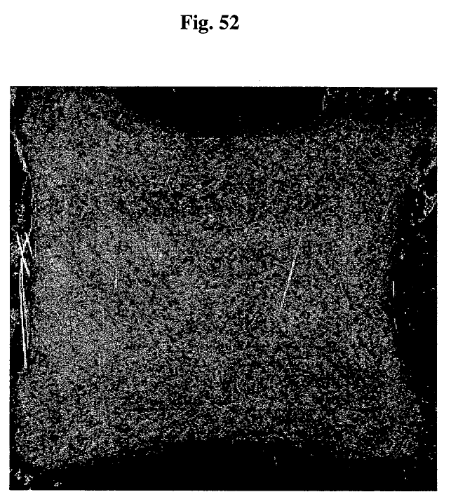

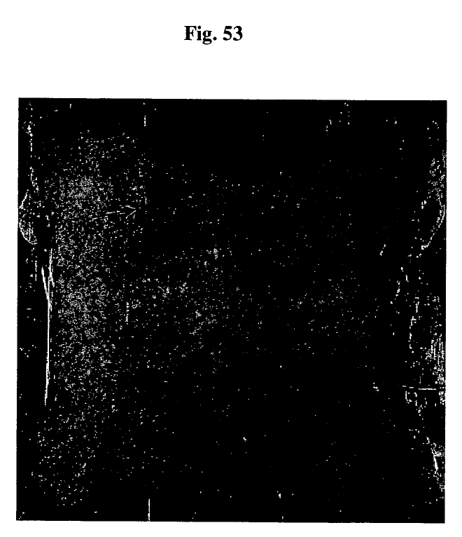

The skin specimen was photographed before the treatment (FIG. 52), immediately after the treatment, and every 15 min for 75 min after the treatment. Carbon particles were removed after the treatment, and the cleaned sample was photographed.

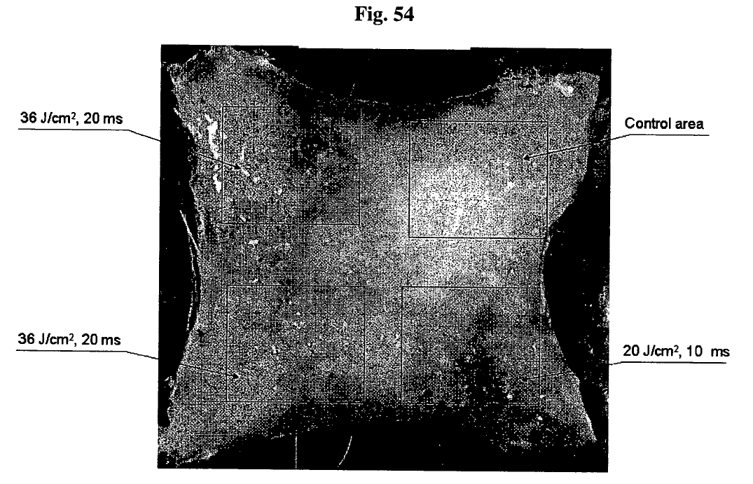

The lattice of damage islets procedure described above created damage islets in the stratum corneum of the farm pig skin specimen that were barely noticeable (FIG. 53). Maximum optical clearance was observed 60 min after the 36 J/cm² light pulses (20 ms). The 36 J/cm² (20 ms) pulses achieved noticeably better clearance than the 20 J/cm² (10 ms) pulses. No detectable clearance was observed in the control (non-treated) area of specimen. (See FIG. 54).

A lattice of thermal damage islets was created in the stratum corneum of the farm pig skin specimen in vitro. The thermal damage islets (i.e., enhanced permeability paths) allowed for superior permeation of the skin by topically applied glucose as evidenced by significantly higher optical clearance than non-treated areas.

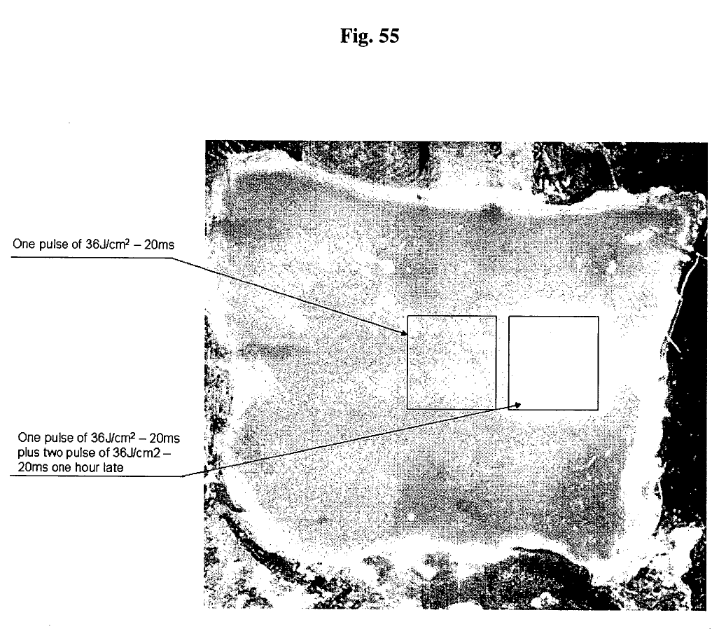

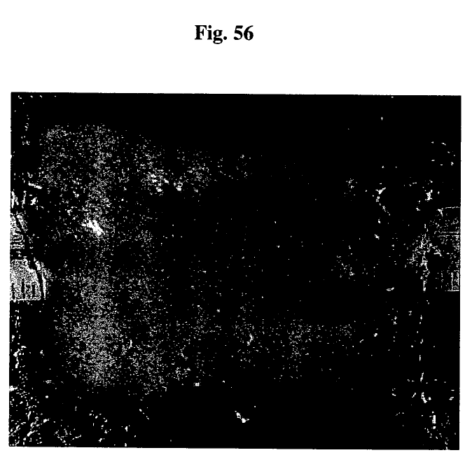

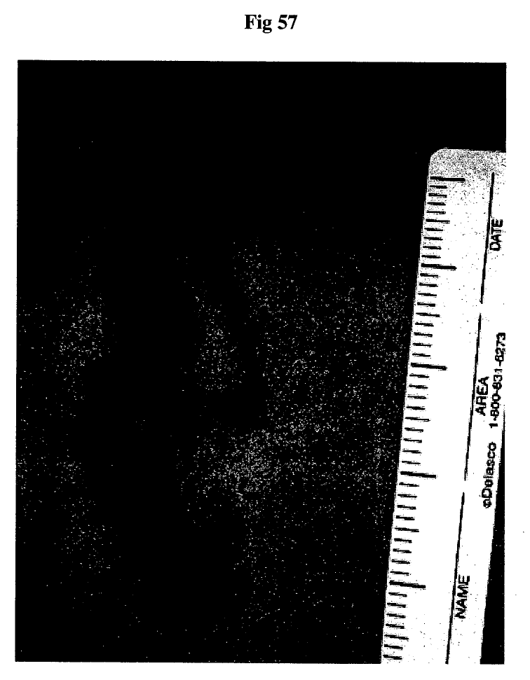

In a second set of experiments, an approximately 4 cm² farm pig skin specimen was glued to a rigid transparent substrate and cleaned with alcohol wipe, and a damage islet mask was placed on the surface of the specimen and covered with a thin layer of lotion, as described above. Two adjacent, approximately 1 cm² areas of specimen received one pulse of 36 J/cm² for 20 ms. Carbon particles were removed after treatment, and the specimen was covered with a 40% solution of glucose in water and maintained at the room temperature for 1 hr. The specimen was warmed up to approximately 40° C. for 2-3 min twice during this period. After one hour, one of the treated areas received two additional pulses of 20 ms at 36 J/cm². Carbon particles were removed and specimen was covered with 40% glucose solution and kept warm using a hair dryer. The surface of the sample was kept wet with fresh glucose solution as needed. Approximately two hours after treatment, optical clearance was assessed by visual observation and documented by photography (see FIGS. 55 and 56).

The specimen area treated with three pulses (20 ms) at 36 J/cm² showed total optical clearance, as compared to no clearance of the non-treated area. The specimen area treated with one pulse (20 ms) at 36 J/cm² showed only partial optical clearance, as compared to no clearance of the non-treated area.

Enhanced Penetration Channels and Optical Clearance of Human Skin In Vivo

A lattice of damage islets was created in the stratum corneum of a human subject in vivo using a flash-arc-lamp system (StarLux Rs™, Palomar Medical Technologies, Burlington, Mass.) and a damage islet mask, as described above. Furthermore, to determine optical clearance of treated areas of skin specimens, a 40% solution of glucose in water was applied to the surface of the specimen.

A tattoo site on a subject's right leg was cleaned with an alcohol wipe and dried. The skin area pre-treatment was photographed (FIG. 57). A flash-arc-lamp system hand piece aperture was covered with a thin layer of lotion (LuxLotion™, Palomar Medical Technologies, Burlington, Mass.) and laser treatment was applied to the selected skin area through the damage islets mask.

A pain tolerance test was performed by applying a series of pulses with incrementally increasing fluence to a selected skin site. The damage islets mask was placed on a dry skin surface and covered with a thin layer of lotion. The pain tolerance test was performed at both the tattooed and non-tattooed sites, and the maximum tolerated fluences were used for the treatments. Two pulses (10 ms) at 10 J/cm², two pulses (10 ms) at 18 J/cm², and two pulses (20 ms) at 24 J/cm² were tested at the tattoo area. Two pulses (20 ms) at 24 J/cm², two pulses (20 ms) at 30 J/cm² and three pulses (20 ms) at 36 J/cm² were tested at the tattooed and non-tattooed skin areas.

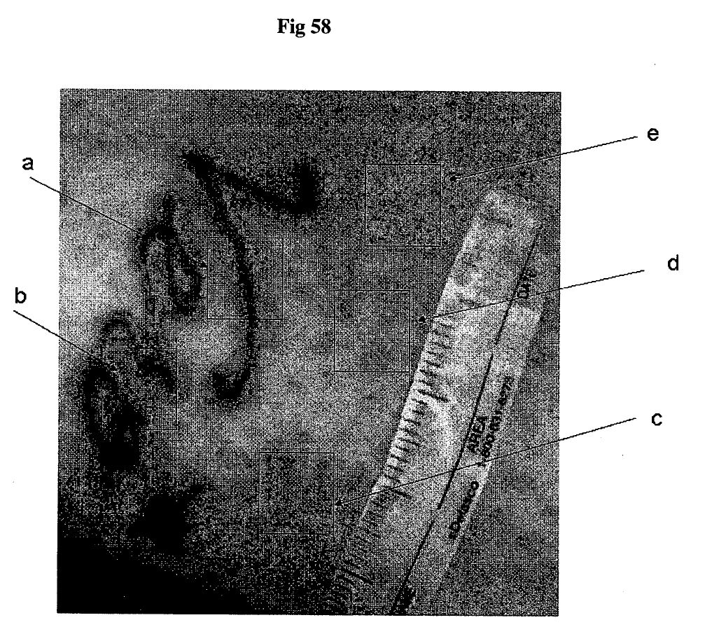

Two different tattoo sites of skin were treated with two pulses (10 ms) at 18 J/cm² two pulses (20 ms) at 24 J/cm². Three different non-tattooed skin sites were treated with two pulses (20 ms) at 30 J/cm², two pulses (20 ms) at 24 J/cm² and three pulses (20 ms) at 36 J/cm². (See FIG. 58). The selected skin sites were cleaned with alcohol wipes and photographed after each treatment.

The subject's tattooed skin area was covered with one layer of a dressing sponge soaked with a 40% solution of glucose in water and kept warm using a hair dryer. The dressing sponge was kept wet by adding fresh glucose solution every 1-2 min, and was replaced every 5 min. The treated area was photographed every 15 min for 90 min. Optical clearance and stratum corneum islets were assessed by visual observation using an optical magnifier.

The subject was provided with glycerin cream for treatment of the tested area. Photos of the treated skin site were taken 6, 9, 24 and 48 hours post treatment. After 48 hours, the skin area was again covered with one layer of dressing sponge, wet with 40% solution of glucose if water, and kept warm by using hair dryer. As before, the dressing sponge was kept wet by adding fresh glucose solution every 1-2 min, and was replaced every 5 min. The treated area was photographed every 20 min for 60 min. Optical clearance and stratum corneum islets were assessed by visual observation using optical magnifier.

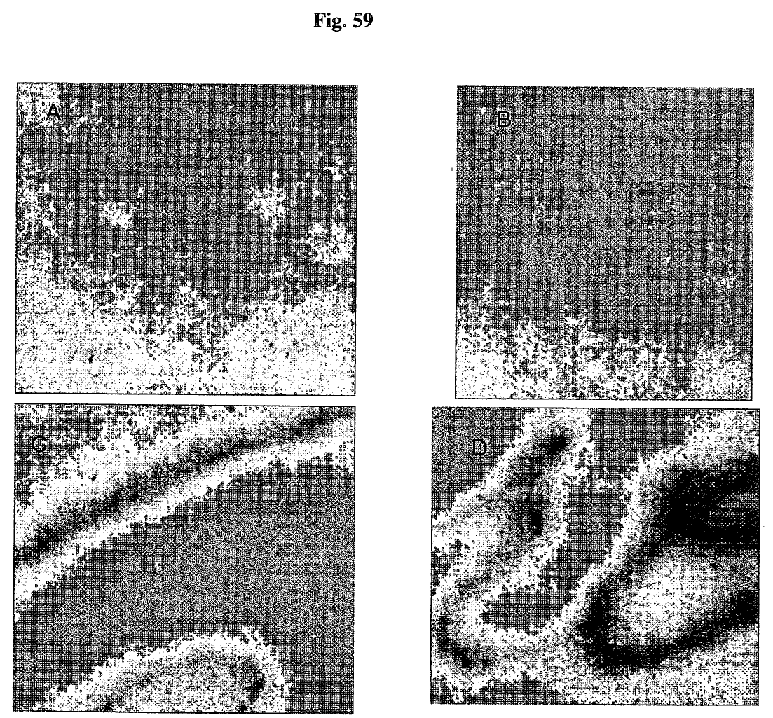

The lattice of damage islets procedure describe above created noticeable damage islets on the stratum corneum of the non-tattooed skin site of the subject after both two pulses (20 ms) at 30 J/cm², and three pulses (20 ms) at 36 J/cm² (FIGS. 59A and 59B). The tattooed area did not show any notable damage islets 90 min after exposure (FIGS. 59C and 59D). No significant optical clearance was observed at any treated areas at the 90 min time point.

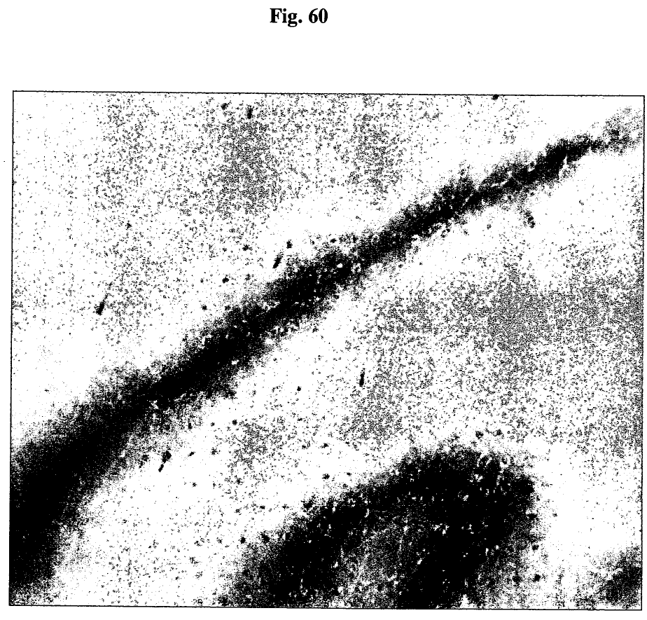

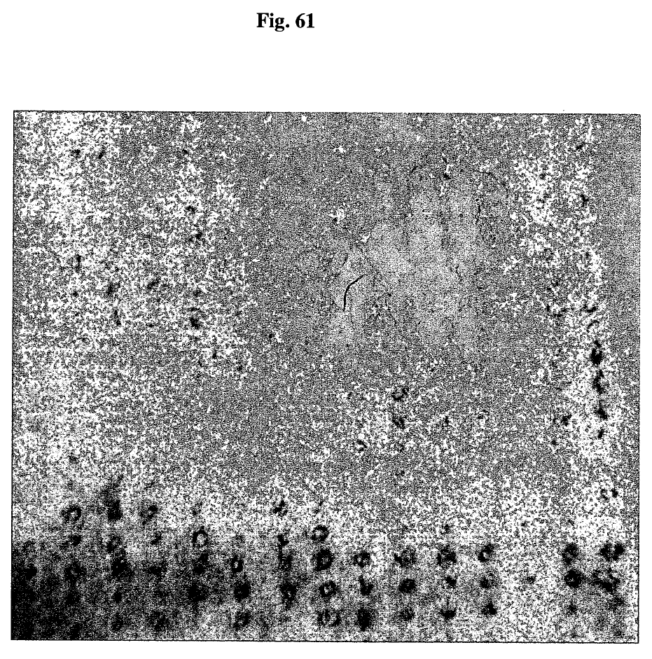

At the 6, 9, 24 and 48 hour time points, the lattice of damage islets became more detectable. The tattooed skin sites became clearly defined at 6 hours after exposure (FIG. 60), and the area treated with three pulses (20 ms) at 36 J/cm² developed edema (FIG. 61).

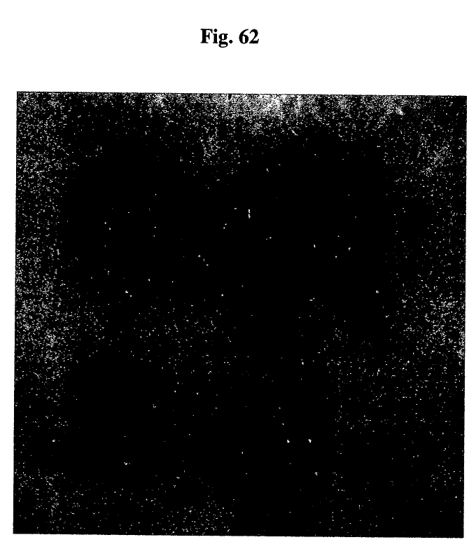

At the 48 hour post-treatment time point, the area treated with three pulses (20 ms) at 36 J/cm² was more red (FIG. 62). The redness was interpreted as enhanced optical clearance due to the application of glycerin cream by the subject, and increased visibility of the vasculature of the dermis. Treatment of the skin site with 40% glucose solution 48 hours after the EMR treatment did not cause any further improvement in optical clearance.

The lattice of damage islets procedure employing three pulses (20 ms) at 36 J/cm² for normal skin and two pulses (20) at 24 J/cm² ms for tattooed skin demonstrated a good pain tolerance margin. The method created visually noticeable damage islets in vivo at the selected human skin areas, and the damage islets became more defined over 6 hours. Treatment of damaged islets on human skin in vivo with a glycerin cream of the site subjected to three pulses (20 ms) at 36 J/cm² resulted in optical clearance manifested by increased visibility of the dermal vasculature.

### EXAMPLE 4

Devices and Systems for Producing Islets of Treatment

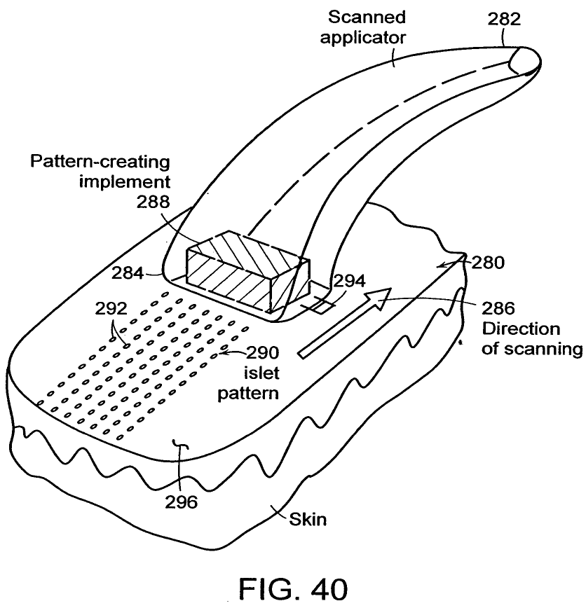

A number of different devices and structures can be used to generate islets of treatment in the skin. FIG. 40 illustrates one system for producing the islets of treatment on the skin 280. An applicator 282 is provided with a handle so that its head 284 can be near or in contact with the skin 280 and scanned in a direction 286 over the skin 280. The applicator 282 can include an islet pattern generator 288 that produces a pattern of areas of enhanced permeability in the SC or arrangement 290 of islets particles 292 on the surface of the skin 280, which when treated with EMR from applicator 210 produces a pattern of enhanced permeability. In other embodiments, the generator 288 produces thermal, damage or photochemical islets into the epidermis or dermis.

In one embodiment, the applicator 282 includes a motion detector 294 that detects the scanning of the head 284 relative to the skin surface 296. This generated information is used by the islet pattern generator 288 to ensure that the desired fill factor or islet density and power is produced on the skin surface 296. For example, if the head 284 is scanned more quickly, the pattern generator responds by imprinting islets more quickly. The following description describes this embodiment of the invention, as well as other embodiments, in greater detail. Further, the following sections elaborate on the types of EMR sources that can be used with the applicator 282 and on the methods and structures that can be used to generate the islets of treatment.

A. Hand Piece with Diode Laser Bar

Some embodiments of the invention use one or more diode laser bars as the EMR source. Because many photodermatology applications require a high-power light source, a standard 40-W, 1-cm-long, cw diode laser bar can be used in some embodiments. Any suitable diode laser bar can be used including, for example, 10-100 W diode laser bars. A number of types of diode lasers, such as those set forth above, can be used within the scope of the invention. Other sources (e.g., LEDs and diode lasers with SHG) can be substituted for the diode laser bar with suitable modifications to the optical and mechanical sub-systems.

FIG. 12A shows one embodiment of the invention using a diode laser bar. Many other embodiments can be used within the scope of the invention. In this embodiment, the hand piece 310 includes a housing 313, a diode laser bar 315, and a cooling or heating plate 317. The housing 313 supports the diode laser bar 315 and the cooling or heating plate 317, and the housing 313 can also support control features (not shown), such as a button to fire the diode laser bar 315. The housing 313 can be made from any suitable material, including, for example, plastics. The cooling plate, if used, can remove heat from the patient's skin. The heating plate, if used, can heat the patient's skin. The same plate can be used for heating or cooling, depending on whether a heat source or source of cooling is applied to the plate.

The diode laser bar 315 can be, in one embodiment, ten to fifty emitters (having widths of 50-to-150 μm in some embodiments or 100-to-150 μm in others) that are located along a 1-cm long diode bar with spacing of 50 to 900 μm. In other embodiments, greater than or less than fifty emitters can be located on the diode laser bar 315, the emitter spacing, and the length of the diode laser bar 315 can also vary. In addition, the width of the emitters can vary. The emitter spacing and the number of emitters can be customized during the manufacturing process.

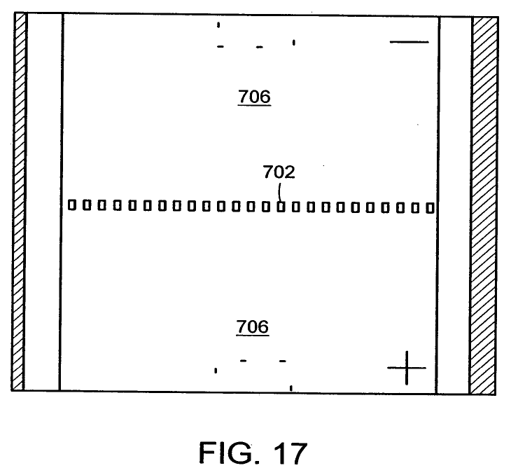

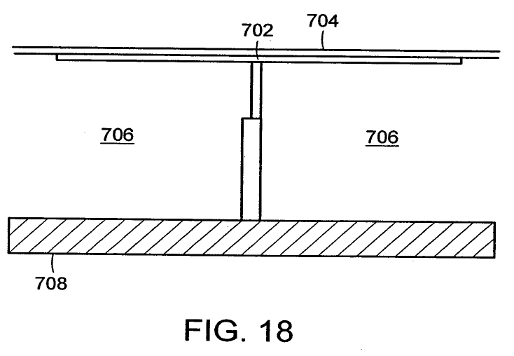

The diode laser bar 315 can be, in one embodiment, twenty-five 100-to-150 μm or 50-to-150 μm wide emitters that are located along a 1 cm long diode bar, each separated by around 50 to 900 microns in some embodiments, and approximately 500 microns in others. FIGS. 17 and 18 depict top and cross-sectional views, respectively, of such a diode laser bar assembly in this embodiment. In this embodiment, twenty-five emitters 702 are located directly beneath the surface plate 704 that is placed in contact with the skin during treatment. Two electrodes 706 are located to each side of the emitters 702. The bottom of the diode assembly contains a cooling agent 708 to control the diode laser and plate 704 temperatures.

In the embodiment of FIGS. 17 and 18, the divergence of the beam emanating from the emitters 702 is between 6 and 12 degrees along one axis (the slow axis) and between 60 and 90 degrees along the fast axis. The plate 704 may serve as either a cooling or a heating surface and also serves to locate the emitters 702 in close and fixed proximity to the surface of the tissue to be treated. The distance between the emitters 702 and the plate 704 can be between about 50 and 1000 micrometers, and more particularly between about 100 and 1000 micrometers in some embodiments, in order to minimize or prevent distortion effects on the laser beam without using any optics for low cost and simplicity of manufacture. During use, the distance between the emitters 702 and the patient's skin can be between about 50 and 1000 micrometers, and more particularly 100 and 1000 micrometers in some embodiments. In such embodiments, imaging optics are not needed, but other embodiments could include additional optics to image the emitter surfaces 702 directly onto the tissue surface. In other embodiments, greater than or less than twenty-five emitters can be located on the diode laser bar, and the length of the diode laser bar can also vary. In addition, the width of the emitters and light divergence can vary. The emitter spacing and the number of emitters can be customized during the manufacturing process.

FIG. 12B shows a perspective view of one embodiment of a diode laser bar 330 that can be used for the diode laser bar 315 in FIG. 12A. The diode laser bar 330 has length L of around 1 cm, a width W of around 1 mm, and a thickness T of around 0.0015 mm. The depiction of FIG. 12B shows 12 emitters 332, each of which emits radiation 334 as shown in FIG. 12B. The diode laser bar 330 can be placed within the device 310 of FIG. 12A so that the side S of the diode laser bar 315 is oriented as shown in FIG. 12A. The emitters, therefore, emit radiation downward toward the skin 319 in the embodiment of FIG. 12A.

Referring again to FIG. 12A, the plate 317 can be of any type, such as those set forth above, in which light from an EMR source can pass through the plate 317. In one embodiment, the plate 317 can be a thin sapphire plate. In other embodiments, other optical materials with good optical transparency and high thermal conductivity/diffusivity, such as, for example, diamond, can be used for the plate 317. The plate 317 can be used to separate the diode laser bar 315 from the patient's skin 319 during use. In addition, the plate 317 can provide cooling or heating to the patient's skin, if desired. The area in which the plate 317 touches the patient's skin can be referred to as the treatment window. The diode laser bar 315 can be disposed within the housing 313 such that the emitters are in close proximity to the plate 317, and therefore in close proximity to the patient's skin when in use.

In operation, one way to create islets of treatment is to place the housing 313, including the diode laser bar 315, in close proximity to the skin, and then fire the laser. Wavelengths near 1750-2000 nm and in the 1400-1600 nm range can be used for creating subsurface islets of treatment with minimal effect on the epidermis due to high water absorption. Wavelengths in the 290-10,000 can be used in some embodiments, while in other wavelengths in the 900-10,000 nm range can be used for creating surface and subsurface islets on the skin. Without moving the hand piece across the skin, a series of treatment islets along a line can be formed in the skin. FIG. 40 shows one possible arrangement 290 of islets on the surface of the skin 280 from the use of such a diode laser bar, where the diode laser bar 315 is pulsed as it moves over the skin in direction A of FIG. 12A.

In another embodiment, the user can simply place the hand piece in contact with the target skin area and move the hand piece over the skin while the diode laser is continuously fired to create a series of lines of treatment. For example, using the diode laser bar 330 of FIG. 12B, 12 lines of treatment would appear on the skin (one line for each emitter).

In another embodiment, an optical fiber can couple to the output of each emitter of the diode laser bar. In such an embodiment, the diode laser bar need not be as close to the skin during use. The optical fibers can, instead, couple the light from the emitters to the plate that will be in close proximity to the skin when in use.

FIG. 12C shows another embodiment of the invention, which uses multiple diode laser bars to create a matrix of islets of treatment. As shown in FIG. 12C, multiple diode laser bars can be arranged to form a stack of bars 325. In FIG. 12C, for example, the stack of bars 325 includes five diode laser bars. In a similar manner as set forth above in connection with FIG. 12A, the stack of bars 325 can be mounted in the housing 313 of a hand piece H101 with the emitters very close to a cooling plate 317.

In operation, the hand piece 310 of FIG. 12C can be brought close to the skin surface 319, such that the cooling plate 317 is in contact with the skin. The user can simply move the hand piece over the skin as the diode lasers are pulsed to create a matrix of islets of treatment in the skin. The emission wavelengths of the stacked bars need not be identical. In some embodiments, it may be advantageous to mix different wavelength bars in the same stack to achieve the desired treatment results. By selecting bars that emit at different wavelengths, the depth of penetration can be varied, and therefore the islets of treatment spot depth can also be varied. Thus, the lines or spots of islets of treatment created by the individual bars can be located at different depths.

During operation, the user of the hand piece 310 of FIG. 12A or 12C can place the treatment window of the hand piece in contact with a first location on the skin, fire the diode lasers in the first location, and then place the hand piece in contact with a second location on the skin and repeat firing.

In addition to the embodiments set forth above in which the diode laser bar(s) is located close to the skin surface to create islets of treatment, a variety of optical systems can be used to couple light from the diode laser bar to the skin. For example, with reference to FIGS. 12A and 12C, imaging optics can be used to re-image the emitters onto the skin surface, which allows space to be incorporated between the diode laser bar 315 (or the stack of bars 325) and the cooling plate 317. In another embodiment, a diffractive optic can be located between the diode laser bar 315 and the output window (i.e., the cooling plate 317) to create an arbitrary matrix of treatment spots. Numerous exemplary types of imaging optics and/or diffractive optics that can also be used in this embodiment of the invention are set forth in the section entitled Devices and Systems for Creation of Islets (Example 2) above.

Another embodiment of the invention is depicted in FIG. 12D. In this embodiment, the housing 313 of the hand piece 310 includes a stack 325 of diode laser bars and a plate 317 as in previous embodiments. This embodiment, however, also includes four diffractive optical elements 330 disposed between the stack 250 and the plate 317. In other embodiments, more or fewer than four diffractive optical elements 330 can be included. The diffractive optical elements 330 can diffract and/or focus the energy from the stack 325 to form a pattern of islets of treatment in the skin 319. In one aspect of the invention, one or more motors 334 is included in the hand piece 310 in order to move the diffractive optical elements 330. The motor 334 can be any suitable motor, including, for example, a linear motor or a piezoelectric motor. In one embodiment, the motor 334 can move one or more of the diffractive optical elements 330 in a horizontal direction so that those elements 330 are no longer in the optical path, leaving only one (or perhaps more) of the diffractive optical elements 334 in the optical path. In another embodiment, the motor 334 can move one or more of the diffractive optical elements 330 in a vertical direction in order to change the focusing of the beams.

In operation, by incorporating more than one diffractive optics 330 in the hand piece 310 along with a motor 334 for moving the different diffractive optics 330 between the stack 325 of diode laser bars and the plate 317, the diffractive optics 330 can be moved in position between the stack 325 and the cooling plate 317 in order to focus the energy into different patterns. Thus, in such an embodiment, the user is able to choose from a number of different islets of treatment patterns in the skin through the use of the same hand piece 310. In order to use this embodiment of the invention, the user can manually place the hand piece 310 on the target area of the skin prior to firing, similar to the embodiments described earlier. In other embodiments, the hand piece aperture need not tough the skin. In such an embodiment, the hand piece may include a stand off mechanism (not shown) for establishing a predetermined distance between the hand piece aperture and the skin surface.

FIG. 12E shows another embodiment of the invention. In this embodiment, optical fibers 340 are used to couple light to the output/aperture of the hand piece 310. Therefore, the diode laser bar (or diode laser bar stacks or other light source) can be located in a base unit or in the hand piece 310 itself. In either case, the optical fibers couple the light to the output/aperture of the hand piece 310.

In the embodiment of FIG. 12E, the optical fibers 340 may be bonded to the treatment window or cooling plate 317 in a matrix arrangement with arbitrary or regular spacing between each of the optical fibers 340. FIG. 12E depicts five such optical fibers 340, although fewer or, more likely, more optical fibers 340 can be used in other embodiments. For example, a matrix arrangement of 30 by 10 optical fibers could be used in one exemplary embodiment. In the depicted embodiment, the diode laser bar (or diode laser bar stacks) is located in the base unit (which is not shown). The diode laser bar (or diode laser bar stacks) can also be kept in the hand piece. The use of optical fibers 340 allow the bar(s) to be located at an arbitrary position within the hand piece 310 or, alternatively, outside the hand piece 310.

As an example of an application of a diode laser bar to create thermal damage zones in the epidermis of human skin, a diode laser bar assembly, as depicted in FIGS. 17 and 18, emitting at a wavelength λ=1.47 μm, was constructed and applied to human skin ex vivo at room temperature in a stamping mode (that is, in a mode where the assembly does not move across the skin during use). The diode bar assembly had a sapphire window, which was placed in contact with the skin and the laser was pulsed for about 10 ms. The treated skin was then sliced through the center of the laser-treated zones to reveal a cross-section of the stratum corneum, epidermis and dermis. The resulting thermal damage channels were approximately 100 μm in diameter and 125-150 μm in depth for the 10 mJ per channel treatments.

B. Hand Piece with Speed Sensor

According to one embodiment of the invention, an apparatus can include a light emitting assembly for applying optical energy to the target area of the patient's skin, a sensor for determining the speed of movement of the head portion across the target area of the patient's skin, and circuitry in communication with the sensor for controlling the optical energy in order to create islets of treatment. The circuitry can control, for example, pulsing of the optical energy source based on the speed of movement of the head portion across the skin in order to create islets of treatment. In another embodiment, the circuitry can control movement of the energy source within the apparatus based on the speed of movement of the head portion across the skin in order to treat certain areas of the skin, while not exposing other areas, in order to create islets of treatment.

FIG. 15 is a bottom view of an embodiment of the invention that includes a speed sensor for measuring the speed of movement of the hand piece across the patient's skin. The embodiment of FIG. 15 can be used, for example, in the embodiment of FIG. 12A. That is, the hand piece 310 of FIG. 12A can include a housing 310, a diode laser bar 315 (or more than one diode laser bars as in FIG. 12C), and a plate 317. FIG. 15 shows a bottom view of a hand piece in which it is equipped with a speed sensor 350, 352.

A number of types of speed sensors can be used to measure the hand piece speed relative to the skin surface. For example, the speed sensor can be an optical mouse, a laser mouse, a wheel/optical encoder, or a capacitive imaging array combined with a flow algorithm similar to the one used in an optical mouse. A capacitive imaging array can be used to measure both hand piece speed and to create an image of the treated area. Capacitive imaging arrays are typically used for thumbprint authentication for security purposes. However, a capacitive imaging array can also be used to measure the hand piece speed across the skin surface. By acquiring capacitive images of the skin surface at a relatively high frame rate (for example, 100-2000 frames per second), a flow algorithm can be used to track the motion of certain features within the image and calculate speed.

In the embodiment of FIG. 15, two capacitive imaging arrays 350, 352 are located on the bottom of the hand piece, with one on each side of the treatment window 354. The diode laser bar 356 output is directed through the treatment window, that is, through a cooling plate or the like. The orientation of the capacitive imaging arrays 350, 352 can vary in different embodiments of the invention. As the device is moved, both capacitive imaging arrays 350, 352 measure the speed of the hand piece across the patient's skin. The configuration can include circuitry that is in communication with the capacitive imaging arrays 350, 352 to measure the speed and determine an appropriate rate for firing the light source (e.g., diode laser) based on that speed. The circuitry, therefore, can also be in communication with the laser in order to pulse the laser at an appropriate speed. The speed sensor incorporated in the hand piece, therefore, can provide feedback to the laser pulse generator. In some embodiments, after an initial pulse of radiation, the pulsing of the diode laser bar 356 might not be enabled until the capacitive imaging arrays 350, 352 sense movement of the hand piece over the skin. This circuitry can be located in the hand piece in some embodiments or, in other embodiments, in a base unit. When the diode laser bar 356 is enabled for firing by the user (for example by depressing a footswitch), a laser pulse generator for the laser fires the laser at a rate proportional to the hand piece speed.

In operation, the embodiment described above can be used to create a uniform matrix of treatment islets by manually moving a hand piece that includes a single diode laser bar (or multiple diode laser bars) across the skin surface and pulsing the laser at a rate proportional to the hand piece speed. For example, decreasing the time interval between laser pulses as the hand piece speed increases can be used to keep a constant matrix of lines of islets of treatment on the skin. Similarly, increasing the time interval between laser pulses as the hand piece speed decreases can be used to keep a constant matrix of lines of islets of treatment on the skin. The treatment head, including treatment window or light aperture of the hand piece, can be rotated to vary the spacing between islets of treatment in the direction orthogonal to hand piece movement.

In addition to measuring hand piece speed, the capacitive imaging arrays 350, 352 can also image the skin after the line of islets of treatment has been created in order to view the treatment results. Acquired images can be viewed in real time during treatment. The hand piece can include, for example, a display that shows the treatment area of the skin under the cooling plate. Alternatively, the acquired images can be stored in a computer for viewing after the treatment is complete. In some embodiments, the system can be configured to display images from both sensors, so that the hand piece can be moved either forward or backward.

In the configurations discussed above, the diode laser is used at a relatively low duty cycle because the laser is turned off in between islets of treatment. In some embodiments of the invention, the diode laser can be used more efficiently by keeping the diode laser on for a longer time, for example, if the of islets of treatment are lines instead of spots. FIG. 16 depicts an example of a hand piece 310 in which the diode laser bar 315 can be mounted on a miniature linear translator 372 inside the hand piece. The hand piece 310 of FIG. 16 can be largely the same as the embodiments set forth above. That is, it can include a diode laser bar 315 adjacent a plate 317 in a hand piece. This embodiment, however, also include a miniature linear translator 372 that can move the diode laser bar 315 in the forward or backward direction within the hand piece 310. Other suitable motors, such as, for example, a piezoelectric motor or any type of linear motor, can be used instead of the miniature linear translator 372. In alternative embodiments, the diode laser bar 315 can be mounted on a cylindrical shaft that can be rotated to accomplish the same function as the linear translator 372. A single-axis galvanometer-driven mirror can also be used.

In the embodiment of FIG. 16, as the hand piece 310 is moved forward (left in the Figure), the diode laser bar 315 would be moved backward (right in the Figure) within the hand piece at the same speed. After the diode laser bar 315 reaches the rear of the hand piece 310, it would be moved to the front of the hand piece, and the cycle would be repeated. The spacing between the lines of islets of treatment can be adjusted by varying the time required to move from the rear to the front of the hand piece 310. In this embodiment, for example, a speed sensor can measure the speed of movement of the hand piece 310 across the skin. This speed sensor can be similar to those described above. Such a speed sensor can be in communication with circuitry that moves the diode laser bar 315 (through the motor 372) based on the speed of the hand piece 310 across the skin. Thus, by appropriately moving the diode laser bar 315 within the hand piece 310, a matrix of treatment islets can be created on the patient's skin.

FIGS. 41A and 41B illustrate another embodiment of the invention that includes a speed sensor. In this embodiment, the hand piece 400 includes a non-coherent EMR source 404 disposed within the housing 402 of the hand piece 400. The non-coherent EMR source 404 can be any of the types set forth above, including, for example, a linear flash lamp, an arc lamp, an incandescence lamp, or a halogen lamp. In one embodiment, the light source 404 is a Xe-filled linear flash lamp.

The hand piece 400 can also include an optical reflector 406, one or more optical filters 408, and a light duct 410 (or concentrator). The optical reflector 530 can serve to reflect and direct the light into the concentrator 410. The concentrator 410 can be made from glass BK7, and can have a trapezoidal shape. In other embodiments, the concentrator 410 can be made from different materials and its shape can vary. The concentrator 410 can be used, for example, for homogenization of the beam. In some embodiments, the optical filter 408 might not be used. If used, the filter 408 can serve to filter out certain wavelengths of light from the EMR source 404. In addition, the optical reflector 406 might not be used in some embodiments. In some embodiments, a cooling plate (not shown in FIGS. 41A and 41B) can be attached to the housing 402 or at the end of the optical path in order to cool the patient's skin.

The housing 402 can be equipped with a speed sensor 412. This speed sensor C560 can measure the speed of movement of the housing 402 with respect to the patient's skin. In the embodiment of FIGS. 41A and 41B, the housing 402 of the hand piece 400 is capable of movement independently from the light source 404 within the housing 402. That is, when the housing 402 moves with a speed V with respect to the patient's skin, the light source 404 can move within the housing 402 such that the light source 404 remains fixed with respect to the patient's skin. That is, the speed v of the light source 404 with respect to the patient's skin is approximately zero, which means that the light source 404 would move relative to the housing and within the housing at a speed of −V. In this embodiment, the light source 404 does not move and is held steady during application of radiation in order to guarantee the desired energy exposure. When treatment of the selected part of skin has been completed, the light source 404 can move within the housing 402 in order to reach its initial position. That is, the light source 404 can move forward in a leap-frog manner with a speed v>V (where both v and V are measured relative to the patient's skin) for treatment of the next part of skin. Such a leap-frog motion is set forth in FIG. 41B.

As set forth above, for synchronization of the speed V of the housing 402 and the speed v of the light source 404, the housing 402 is equipped with the speed sensor 412. The speed sensor 412 can measure the movement of the housing 402 with respect to the patient's skin and then move the light source 404 within the housing 540210 at an appropriate speed in order to remain fixed with respect to the patient's skin. The hand piece 400 or a base unit associated with the hand piece 400 can include circuitry that receives the speed of movement of the housing 402 and then sends a signal to a motor that moves the light source 404 within the housing 402 at an appropriate speed. The hand piece 400, therefore, can include a linear motor or linear translator, such as those set forth above, to move the light source 404 within the housing 402.

The description above indicates that the light source 404 is moveable within the housing 402. The reflector 406, the filter 408, and the concentrator 412, if used, can be connected to the light source 404 in some embodiments in a manner so that these components move within the housing 402 along with the light source 404. FIG. 41B depicts an embodiment in which these components move along with the light source 404.

In some embodiments using a Xe-filled linear flash lamp, the spectral range of the EMR is 300-3000 nm, the energy exposure up to 1000 J/cm², the pulse duration is from about 0.1 ms to 10 s, and the fill factor is about 1% to 90%.

Another embodiment of the invention involves the use of imaging optics to image the patient's skin and use that information to determine medication application rates, application of EMR, or the like in order to optimize performance. For instance, some medical or cosmetic skin treatments require that the medication application rate be accurately measured and its effect be analyzed in real time. The skin surface imaging system can detect the size of reversible or irreversible holes created with techniques proposed in this specification for creating treatment islets in the stratum corneum. For this purpose, a capacitive imaging array can be used in combination with an image enhancing lotion and a specially optimized navigation/image processing algorithm to measure and control the application rate.

The use of a capacitive imaging array is set forth above in connection with FIG. 15. Such capacitive image arrays can be used, for example, within the applicator 282 of FIG. 40 according to this embodiment of the invention. As set forth above, in addition to measuring hand piece speed, the capacitive imaging arrays 350, 352 (FIG. 15) can also image the skin. Acquired images can be viewed in real time during treatment via a display window of the device.

One example of a suitable capacitive sensor for this embodiment of the invention is a sensor having an array of 8 image-sensing rows by 212 image-sensing columns. Due to inherent limitations of capacitive array technology, a typical capacitive array sensor is capable of processing about 2000 images per second. To allow for processing skin images in real time, an orientation of the sensor can be selected to aid in functionality. In one embodiment, for instance, the images are acquired and processed along the columns. This allows for accurate measurement of velocity up to about 200 mm/s.

For the sensor to function reliably and accurately, the skin surface can be treated with an appropriate lotion. The selection of the lotion can be important to the light-based skin treatment and navigation sensor operation. The lotion should be optically transparent to the selected wavelength, provide image enhancement to a sensor, and function as a friction reduction lubricant.

Circuitry containing a processing algorithm or the like can be in communication with the capacitive image sensor. The capacitive sensor and its associated processing algorithm are capable of determining a type of lotion and its effect on the skin surface. This can be performed in real time by sequentially analyzing the image spectral characteristics. The processing algorithm can also perform sensor calibration, image contrast enhancement, and filtering, as well as processing and control of images of the skin surface with navigation code to aid in various applications.

Real time acquired images can be used for statistical analysis of a marker concentration in a lotion. The markers are put in a lotion to function as identifiers of a treatment area. The marker can be a chromophore itself (i.e., a chromophore that heats up upon application of irradiation) or it can be a chemical that indicates the presence of the chromophore or medication in the lotion. As one example, the marker emits or reflects light proportional to the incident light to indicate the concentration of a chromophore or medication in the lotion. The capacitive sensor, therefore, can function to determine whether the marker concentration of a given lotion is at an appropriate level. The circuitry can, for instance, send a signal to the user of the concentration of the marker. Alternatively, the circuitry can determine if the marker concentration meets a preselected set point concentration level for a certain marker. If the set point is not met, the circuitry can communication to the user to let the user know that more (or perhaps less) lotion may be needed on the patient's skin. Selected markers with the right lotion pH level can also be used as an eye safety enhancement feature for light treatment on human body.

The sensor can also function as a contact sensor. This allows for real time determination of immediate contact of a hand piece with the patient's skin. The combination of hardware and software allows this determination within one image frame. The algorithm measures in real time a skin contact and navigation parameters (position, velocity and acceleration) along the x-axis and y-axis. This enhances the safety of light treatment on human skin by allowing for the control of the velocity and the quality of skin contact. The quality of contact can be a function of lotion type and pressure applied to the treatment device.

The capacitive sensor along with image processing and special lotion can be used for detecting a skin imperfection and measuring its size in real time. The resolution of the sensor will depend on pixel size, image processing and the sub pixel sampling.

The capacitive sensor and image processing allow for determination of whether the device is operating on biological skin or some form of other surface. It is possible under proper sampling conditions to extract the type of skin the device is moving across. This is accomplished by comparing real time processed images to a stored pattern or calculated set of parameters. In addition, the combination of the capacitive sensor and image pattern recognition, navigation algorithm, and special lotion, can be used to determine the presence of skin hair and provide statistical information about the density and size of the hair.

The capacitive sensor with a combination of two types of lotion, a calibrated skin penetration lotion and image enhancing lotion, can determine the effect of skin rejuvenation on skin over a large area. This analysis can be performed in real time by treating the skin with two lotions and then moving the capacitive sensor over the skin area of interest. The real time algorithm determines the effective area of treatment and the enhancement factor above the norm.

C. Mirror with Holes

FIGS. 7 and 8 illustrate embodiments of the invention in which the islets of treatment are formed generally through the use of a mirror containing holes or other transmissive portions. Light passes through the holes in the mirror and strikes the patient's skin, creating islets of treatment. Light reflected by the mirror stays in the optical system and through a system of reflectors is re-reflected back toward the mirror which again allows light to pass through the holes. In this manner, the use of a mirror containing holes can be more efficient than the use of a mask with holes, where the mask absorbs rather than reflects light.

In the embodiment of FIG. 7, the patterned optical radiation to form the islets of treatment is generated by a specially designed laser system 420 and an output mirror 422. The laser system 420 and output mirror 422 can be contained in, for instance, a hand piece. In other embodiments, the laser system 420 can be contained in a base unit and the light passing through the holes in the mirror can be transported to the hand piece aperture through a coherent fiber optic cable. In still other embodiments, the laser can be mounted in the hand piece and microbeams from the laser can be directed to the skin with an optical system. In the illustrated embodiment, the laser system 420 comprises a pump source 426, which optically or electrically pumps the gain medium 428 or active laser medium. The gain medium 428 is in a laser cavity defined by rear mirror 430 and output mirror 422. Any type of EMR source, including coherent and non-coherent sources, can be used in this embodiment instead of the particular laser system 420 shown in FIG. 7.

According to one aspect of the invention, the output mirror 422 includes highly reflective portions 432 that provide feedback (or reflection) into the laser cavity. The output mirror 422 also includes highly transmissive portions 434, which function to produce multiple beams of light that irradiate the surface 438 of the patient's skin 440. FIG. 7 depicts the highly transmissive portions 434 as being circular shapes, although other shapes, including, for example, rectangles, lines, or ovals, can also be used. The transmissive portions 434 can, in some embodiments, be holes in the mirror. In other examples, the transmissive portions 434 include partially transparent micro mirrors, uncoated openings, or openings in the mirror 422 with an antireflection coating. In these embodiments, the rest of the output mirror 422 is a solid mirror or an uncoated surface.

In one implementation, the output mirror 422 functions as a diffractive multi-spot sieve mirror. Such an output mirror 422 can also serve as a terminal or contact component of the optical system 420 to the surface 438 of the skin 440. In other embodiments, the output mirror 422 can be made from any reflective material.

Because of the higher refractive index of the illuminated tissue of the skin 440, divergence of the beams is reduced when it is coupled into the skin 440. In other embodiments, one or more optical elements (not shown) can be added to the mirror 422 in order to image a sieve pattern of the output mirror 422 onto the surface of the skin 440. In this latter example, the output mirror 422 is usually held away from the skin surface 438 by a distance dictated by the imaging optical elements.

Proper choice of the laser cavity length L, operational wavelength X of the source 426, the gain g of the laser media 428, dimensions or diameter D of the transmissive portions 434 (i.e., if circular) in the output mirror 422, and the output coupler (if used) can help to produce output beams 436 with optimal properties for creating islets of treatment. For example, when D2/4λL<1, effective output beam diameter is made considerably smaller than D, achieving a size close to the system's wavelength λ of operation. This regime can be used to produce any type of treatment islets.

Typically, the operational wavelength ranges from about 0.29 μm to 100 μm and the incident fluence is in the range from 1 mJ/cm² to 100 J/cm². The effective heating pulse width can be in the range of less than 100 times the thermal relaxation time of a patterned compound (e.g., from 100 fsec to 1 sec).

In other embodiments, the chromophore layer is not used. Instead the wavelength of light is selected to directly create the pathways.

In one example, the spectrum of the light is in the range of or around the absorption peaks for water. These include, for example, 970 nm, 1200 nm, 1470 nm, 1900 nm, 2940 nm, and/or any wavelength>1800 nm. In other examples, the spectrum is tuned close to the absorption peaks for lipids, such as 0.92 μm, 1.2 μm, 1.7 μm, and/or 2.3 μm, and wavelengths like 3.4 μm, and longer or absorption peaks for proteins, such as keratin, or other endogenous tissue chromophores contained in the SC.

The wavelength can also be selected from the range in which this absorption coefficient is higher than 1 cm⁻¹, such as higher than about 10 cm⁻¹. Typically, the wavelength ranges from about 0.29 μm to 100 μm and the incident fluence is in the range from 1 mJ/cm² to 1000 J/cm². The effective heating pulse width is preferably less than 100× thermal relaxation time of the targeted chromophores (e.g., from 100 fsec to 1 sec).

The embodiment of FIG. 7 can be used to create islets of treatment in the stratum corneum. Controlling permeability of the stratum corneum can also be accomplished by absorption, scattering, or refractive coupling. Skin or topical cooling can be applied to prevent SC damage between the pathways and to control their size.

FIG. 8 depicts a second embodiment of a hand piece 450 that uses a mirror in order to reflect portions of EMR, while allowing certain patterns of the EMR to pass through holes in order to create islets of treatment. The embodiment of FIG. 8 includes a light source 452 and, in some embodiments, beam-shaping optics 454 and a waveguide 456. These components can be in a hand piece 450, such as those hand pieces set forth above. In other embodiments, the light source 452 can be in a base unit outside of the hand piece 450. The light source 452 can be a laser, a flashlamp, a halogen lamp, an LED, or another coherent or thermal source. In short, the light source 452 can be any type of EMR source as set forth above. The beam-shaping optics 454 can be reflective or refractive and can serve to direct EMR downward toward the output of the hand piece. The beam-shaping optics 454 can generally be disposed above and to the sides of the light source 452. The waveguide 456 can be used, for example, for homogenization of the beam 458.

The hand piece 150 of the embodiment of FIG. 8 can also include an output window 460 near the optical output from the hand piece 450. The output window 460 can be coated with a generally non-transparent coating. The coating can be, for instance, a reflective coating, such as a multi-layer dielectric coating. Such a dielectric coating can be selected to have a high reflectance over a spectral band defined by the EMR source 452. If selected to be highly reflective, such a dielectric coating will not absorb a large amount of light causing it to heat up. In addition, the window with the dielectric coating can be cooled if necessary for heat removal from the skin. Such a dielectric coating can be fabricated by vacuum deposition of one or, more likely, multiple dielectric layers. In some embodiments, the output window 460 can be made from a lattice of microlenses that serves to provide spatial modulation of the power density in the lattice of optical islets.

The coating of the output window 460 can have a number of openings (or holes or transmissive portions) 462, which reshape the output beam into a plurality of beamlets 464. The openings 464 can be coated with anti-reflective coatings, or can contain Fresnel or refractive lenses for angular beam shaping. The openings 464 do not necessarily have to be of circular shape, as depicted in FIG. 8. The shape of the openings 464 can be adjusted depending on the skin condition to be treated. For example, the openings 464 can be circular, slits, rectangles, ovals, lines, or irregular shapes. In some embodiments, the shape of the openings 464 can be changed on demand (adaptively) depending on underlying skin conditions by using, for example, an electro-optical or thermo-optical effect.

The device can contain a cooling implement 466 to provide active contact cooling to the treatment area. The cooling implement 466 can be, for example, a sapphire cooling plate that is cooled by a water manifold or the like built into the hand piece, as set forth above. In addition, any other type of cooling implement 466, such as those set forth above, can be used.

The device of the embodiment of FIG. 8 can also include a device for monitoring the temperature of the waveguide 456 and/or the patient's skin 470. The temperature monitoring can be done, for example, using an optical device. In such an embodiment, a separate optical source 472 can be used to shine a probing beam 474 onto the output window 460. The reflected light is then detected with a detector 476. When the refractive indices of the layers in the multi-layer dielectric coating (or mirror or output window 460) change as a result of temperature change, the reflection coefficient of the coating changes as well. Thus, a temperature change can be deduced from the reflection measurements. A section 478 of the output window 460 can be optically separated from the skin 470 in order to reduce background parasitic signal from the skin 470 in measuring the temperature of the output window 460. The optical source 472 and the detector 476 can be built into the hand piece.

In some embodiments, the openings 462 in the output window 460 can be coated with phase-changing material, which changes its transparency as a result of temperature change. That is, the transparency of the openings 462 decreases when the temperature increases. Thus, overheating of skin 470 can be prevented by blocking the beamlets 474 with the decreased transparency of the openings 416.

In operation, the output window 460 is brought into contact with the treatment area 470 (i.e., the patient's skin). The light source 452 is then fired to output radiation from the hand piece. The openings 462 in the output window 462 form islets of treatment on the patient's skin 470.

The device of FIG. 8 can be used either in the stamping modes or the sliding modes. A stamping mode is a mode in which the device is placed on the skin and the radiation source is activated while the device remains stationary on the skin. In the sliding mode, the device can be moved over the skin while in contact with the skin. In the stamping modes, the resulting temperature in the skin (and, possibly, the damage profile) is completely determined by the geometry of the openings and the illumination/cooling parameters. In the sliding modes, an additional degree of control is available by varying the velocity of scanning.

The device of FIG. 8 can have an optical coating (i.e., on the treatment window 460) to provide light spatial modulation. Some embodiments can use technology similar to a gradient mirror, which is a mirror with variable transmission over its radius. An embodiment including a plurality of gradient mirrors could be beneficial for enhancement of parameters of the light source (such as the effect of photon recycling) and system cooling capabilities (very thin coating thickness).

In some embodiment, the coating, (such as, for example, a multilayer dielectric high reflective coating with lattice of transparent zones) can be coated directly on the contact cooling surface of a sapphire chilled bock. In such an embodiment, the entire sapphire block can be used as a cooling area, but the irradiated area is limited by the area of the transparent zones. Such an embodiment can be effective for a combination of LOI treatment with skin upper layer protection for deep dermal or fat treatments.

In another embodiment, where a laser source is used, the laser itself can have an output that is not uniform. For example, in such an embodiment, the laser itself can be surrounded by a reflector, which can be a high reflector. The reflector surrounding the laser, and in particular at the output end of the laser, can have areas that are less reflective than other areas. That is, the reflector in such an embodiment does not have uniform reflectivity. These areas can result in increased radiation output from the laser source in discrete areas (or holes). Thus, the reflector or mirror surrounding the laser can itself generated spatially modulated light as an output. The laser source can therefore be housed in a hand piece that has the laser source output close to the output from the hand piece. The hand piece can therefore be brought into close proximity to the skin and fired to create treatment islets.

D. Skin Lifting Implement

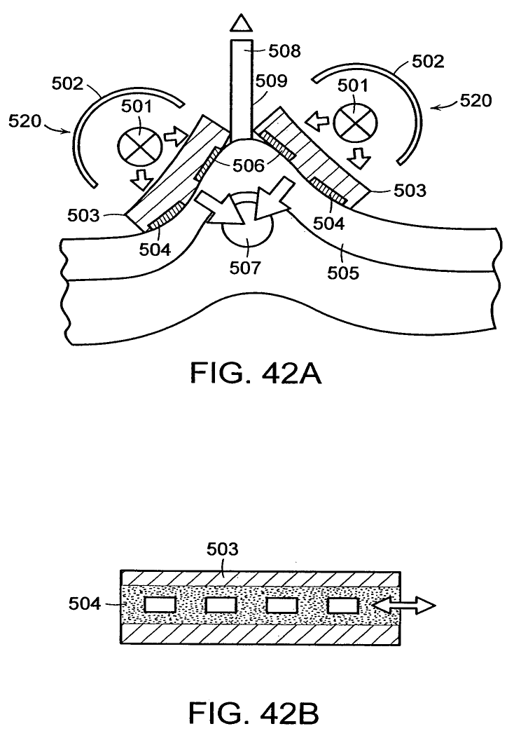

Another embodiment of the invention is illustrated in FIG. 42A. In this embodiment, a hand piece contains two light-emitting assemblies 520 that are positioned at an angle to each other. Each light-emitting assembly 520 includes a light source 501, a beam-shaping implement 502, and an output window 503. The light source 501 can be any variety of EMR source as set forth above. The beam-shaping implement 502 can be a device to reflect and focus EMR from the light source 501. The output window 503 can be a contact plate for the patient's skin that is similar to those contact or cooling plates set forth above.

The skin-lifting implement 508 is used to create a skin fold of the treatment skin area 505. The skin-lifting implement 508 can be, for example, a vacuum implement. Parameters of the illumination (wavelength spectrum, power, cooling, etc.) can be selected in such a way that beamlets 506 of EMR create an area of sufficient irradiance only in one or more limited spatial zones 507 where the beamlets 506 intersect. Thus, the dimensions of the damage zone (or areas with islets of treatment) can be controlled with high precision. The device of FIG. 42A can contain masks 504 with coatings or reflective surface in the output windows 503 similar to those set forth above in connection with FIGS. 7 and 8.

In one embodiment, the mask 504 of each assembly 520 can slide with respect to the corresponding window 503. For example, with reference to FIG. 42B, the mask 504 is movable within the window 503 so that, for example, the mask stays fixed with respect to the patient's skin for a brief period of time when the hand piece moves over the skin. The mask 504, therefore, can slide within the hand piece at a rate proportional to the speed of movement of the hand piece over the patient's skin in a manner as set forth above. Thus, the mutual positions of the beamlets 506 and, therefore, the zones of overlapping beamlets 506, can be controlled with even greater precision to create islets of treatment in the patient's skin. After a brief period of time in which the mask 504 remains fixed with respect to the patient's skin, the mask 504 leap-frogs in position within the output window 503 in order to treat a different area of the patient's skin.

Like the device of FIG. 8, the device of FIG. 42A can be used either in the stamping modes or the sliding modes.

Another implementation can be a vacuum chamber surrounding the treatment area. That is, a vacuum change can surround the distal tip of a hand piece (i.e., the portion in contact with the patient's skin). Such an implementation can be beneficial in increasing the density of treatment islets. The vacuum chamber can laterally stretch the skin and keep it stretched and in contact with distal tip during treatment. After releasing of the skin from the vacuum change the skin will reform back to its initial size with significantly denser islets.

The use of such a vacuum changer surrounding the hand piece distal tip can also increase blood circulation, which can benefit treatment of conditions where hemoglobin is a chromophore. A further increase of the vacuum force can bring the skin into direct contact with the tip of the hand piece and in the contact area internal blood pressure will be relived and blood circulation will decrease. If the chamber design allows skin to stretch laterally outside the tip area, further compression of blood vessels will increase skin transparency to certain wavelengths of light and will increase light penetration depth. Another advance of this concept is that a lower temperature and a lower energy level can be used for stretched skin in order to denature the skin. In addition, stretched skin can result in a lower scattering level and better penetration for light.

E. Hand Pieces with Non-Coherent Light Sources to Form Islets of Treatment

FIG. 9A shows another embodiment of the invention. In this embodiment, the invention is a hand piece 540 that includes an EMR source 542 and a distal end 544 shaped in a manner to create output light spatial modulation and concentration, and therefore to form islets of treatment in a patient's skin. The EMR source 542 can, in some embodiments, be any of the types of non-coherent sources set forth above, including, for example, a linear flash lamp, an arc lamp, an incandescence lamp, or a halogen lamp. In one embodiment, the light source 542 is a Xe-filled linear flash lamp.

The hand piece 540 can also include an optical reflector 546, one or more optical filters 548, and a light duct 550 (or concentrator). The optical reflector 546 can serve to reflect and direct the light into the concentrator 550. The concentrator 550 can be made from BK7 glass, and can have a trapezoidal shape. In other embodiments, the concentrator 550 can be made from different materials and its shape can vary. The concentrator 550 can be used, for example, for homogenization of the beam. In some embodiments, the optical filter 548 might not be used. If used, the filter 548 can serve to filter out certain wavelengths of light from the EMR source 542. In addition, the optical reflector 546 might not be used in some embodiments. In some embodiments, a cooling plate (not shown in FIGS. 9A-E) can be attached to the housing of the hand piece or at the end of the optical path in order to cool the patient's skin.

The distal end 544 of the concentrator 550 can include an array shaped in a manner to create output light spatial modulation and concentration, and therefore to form islets of treatment in a patient's skin. For example, the distal end 544 can include an array of pyramids (FIG. 9B), cones (FIG. 9C), hemispheres (FIG. 9D), grooves (FIG. 9E), prisms, or other structures for output light spatial modulation and concentration. The distal end, therefore, can be made from any type of array, such as micro prisms, that create output modulation and concentration to produce islets of treatment.

In the exemplary embodiment of FIGS. 9A-E using a Xe-filled linear flash lamp, the spectral range of electromagnetic radiation is about 300-3000 nm, the energy exposure is up to about 1000 J/cm², the laser pulse duration is from about 10 ps to 10 s, and the fill factor is from about 1% to 90%.

FIG. 43A shows another embodiment of the invention. In this embodiment, the invention is a hand piece 540 that includes many of the same elements as in the embodiment of FIG. 9A. That is, the embodiment of FIG. 43A can include an EMR source 542, an optical reflector 546, one or more optical filters 548, a light duct 550 (or concentrator), and a cooling plate (not pictured). Each of these components can be similar to or the same as the components set forth above in connection with FIG. 9A.

In the embodiment of FIG. 43A, the distal end 544 of the concentrator 550 can be made as an optically diffusive surface with clear (polished) spots for output light spatial modulation. For example, with reference to FIG. 43B, which shows a side and a top view of the distal end 544, the distal end 544 can include a scattering film 560 with circular openings 570. The scattering film 560 with circular openings 570 can create output modulation to produce islets of treatment on the patient's skin. In particular, the openings 570 (which can be clear, polished spots) can allow for the passage of EMR in order to create the islets of treatment.

FIG. 13A shows another embodiment of the invention. In this embodiment, the invention is a hand piece 540 that includes many of the same elements as in the embodiment of FIGS. 9A and 43A. That is, the embodiment of FIG. 13A can include an EMR source 542, an optical reflector 546, one or more optical filters 548, a light duct 550 (or concentrator), and a cooling plate (not pictured). Each of these components can be similar to or the same as the components set forth above in connection with FIG. 9A.

In the embodiment of FIG. 13A, the light guide 550 can be made from a bundle of optical fibers 580 doped with ions of rear earth metals. For example, the light guide 550 can be made from a bundle of Er³⁺ doped fiber. The active ions inside the light guide core 582 can act as fluorescent (or super fluorescent) converters to provide desired spatial modulation and spectrum conversion. Thus, the light guide 550 in the embodiment of FIG. 13A can create spatial modulation of the EMR in order to create islets of treatment.

FIGS. 13B, 13C, and 13D show embodiments in which the optical fibers 580 are wrapped around the EMR source 542 in order to couple light into the optical fibers 580. As shown in FIG. 13C, each individual fiber or group of fibers 580 can have its output directed to the skin. FIG. 13D shows a bottom view of the output from the hand piece. As shown in FIG. 13D, the fibers 580 can have an output distribution that is spatially modulated in order to create islets of treatment.

FIG. 13E shows another embodiment that uses the same general structure as the embodiments of FIGS. 13A, 13B, and 13C. In the embodiment of FIG. 13E, the output of the fiber bundle 580 (i.e., the bundle of FIGS. 13B-D) can have a distal end that is made from an array of micro lenses 586 attached to the output face of the light guide. The array of micro lenses 586 can serve to focus and concentrate the output from the fiber bundle 580 in order to create islets of damage.

FIG. 11 shows another embodiment of the invention. In this embodiment, the invention includes a hand piece 600 with multiple sets of EMR sources 604, reflectors 602, filters 606, and light guides 608. The output of each light guide can also be a cooling plate. Each of these components can be similar to or the same as the components set forth above in connection with FIG. 9A. In this embodiment, the spacing between the individual EMR sources (emitters) can provide the desired light spatial modulation in order to form islets of treatment. FIG. 11 shows four sets of EMR sources 604 and associated components. In other embodiments, however, more than or less than four sets of EMR sources 604 can be used. In addition, an array of EMR sources can be used in some embodiments. For instance, such an array could be 4 by 6, for a total array of 24 EMR sources.

F. Hand Piece with Total Internal Reflection

FIGS. 10A-10C show another embodiment of the invention in which the output EMR from the hand piece is totally internally reflected when the hand piece is not in contact with a patient's skin. When the hand piece is in contact with a patient's skin, the output EMR is spatially modulated in order to create islets of treatment in the patient's skin.

In the embodiment of FIGS. 10A-10C, the invention is a hand piece 540 that includes many of the same components as in the embodiment of FIGS. 9A-E. That is, the embodiment of FIGS. 10A-10C can include an EMR source 542, an optical reflector 546, one or more optical filters 548, a light duct 550 (or concentrator), and a cooling plate (not pictured). Each of these components can be similar to or the same as the components set forth above in connection with FIG. 9A.

The total internal reflection in the embodiment of FIGS. 10A-10C is caused by the shape of the distal end 544 of the light duct 550. The distal end 550 can be an array of prisms, pyramids, hemispheres, cones, etc . . . , such as set forth in FIGS. 10B and 10C. The array of elements have dimensions and shapes that introduce light total internal reflection (TIR) when the distal end 544 is in a contact with air, as shown in FIG. 10B. In contrast, the distal end 544 does not cause TIR (it frustrates TIR) when the distal end 544 is in a contact with a lotion or skin surface, as shown in FIG. 10C. Further, when the distal end 544 is in a contact with a lotion or skin surface, this leads to light spatial modulation and concentration of the EMR in a contact area of the patient's skin, causing islets of treatment.

In the exemplary embodiment of FIG. 10A-10C using a Xe-filled linear flash lamps, the spectral range of electromagnetic radiation is about 300-3000 nm, the energy exposure is up to about 1000 J/cm², the laser pulse duration is from about 0.1 ms to 10 seconds, and the fill factor is from about 1% to 90%.

The embodiments of FIGS. 10A, 10B, and 10C depict the use of a non-coherent light source in a hand piece. However, a mechanism can also be used to cause TIR in an embodiment using a coherent light source, such as, for example, a solid state laser or a diode laser bar. Referring to the embodiments of FIGS. 12A-E, 15 and 16, the light from the diode laser bar 315 (in FIG. 12A) can also be coupled to the skin via a total internal reflection (TIR) prism. Since the diode laser bar 315 might not be located in close proximity to the skin surface, an optical system might be required to re-image the emitters onto the skin. Thus, a distal end with prisms or the like can be used to re-image the emitters onto the skin. In one embodiment, a TIR prism can be used. When the TIR prism is not in contact with patient's skin, light from the diode laser bar would be internally reflected and no light would be emitted from the hand piece. However, when the patient's skin is coated with an index-matching lotion and the skin is brought into contact with the hand piece (and, in particular, the prism), light is coupled into the skin. Thus, in a manner similar to that described above for non-coherent light sources, TIR reflection prisms or arrays can also be used in embodiments using coherent light sources. This feature can be important for eye and skin safety.

G. Solid State Laser Embodiments

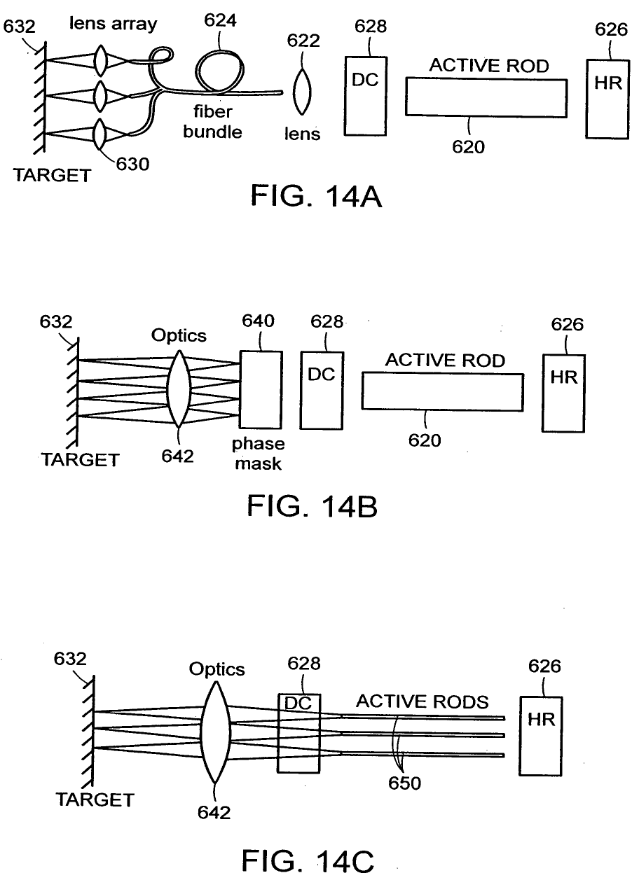

FIGS. 14A, 14B, and 14C show additional embodiments of the invention. FIG. 14A shows an embodiment in which the apparatus includes a laser source 620, focusing optics (e.g., a lens) 622, and a fiber bundle 624. The laser source 620 can be any suitable source for this application, for example, a solid state laser, a fiber laser, a diode laser, or a dye laser. In one embodiment, the laser source 620 can be an active rod made from garnet doped with rare earth ions. The laser source 620 can be housed in a hand piece or in a separate base unit.

In the exemplary embodiment as in FIG. 14A, the laser source 620 is surrounded by a reflector 626 (which can be a high reflector HR) and an output coupler 628 (OC). In other embodiments, the reflector 626 and the coupler 628 are not used. Various types and geometries of reflectors can be used for reflector 626. The fiber bundle 624 is located optically downstream from the lens 622, so that the optical lens 622 directs and focuses light into the fiber bundle 624.

In one embodiment, an optical element 630, such as a lens array, can be used to direct and output the EMR from the fiber bundle 624 in order to focus the EMR onto the patient's skin 632. The optical element 630 can be any suitable element or an array of elements (such as lenses or micro lenses) for focusing EMR. In the embodiment of FIG. 14A, the optical element 630 is a micro lens array. In other embodiments, an optical element 630 might not be used. In such an embodiment, the outputs of the fibers in the fiber bundle 624 can be connected to one side of a treatment window (such as a cooling plate of the apparatus), where the other side of the treatment window is in contact with the patient's skin 632.

In operation, the laser source 620 generates EMR and the reflector 626 reflects some of it back toward the output coupler 628. The EMR then passes through the output coupler 628 to the optical lens 622, which directs and focuses the EMR into the fiber bundle 624. The micro lens array 630 at the end of the fiber bundle 624 focuses the EMR onto the patient's skin 632.

FIG. 14B shows another embodiment of the invention. In this embodiment, the apparatus includes a laser source 620 and a phase mask 640. The laser source 620 can be any type of laser source and can be housed in a hand piece or in a separate base unit, such as in the embodiment of FIG. 14A. In one embodiment, the laser source 620 can be an active rod made from garnet doped with rare earth ions. Also like the embodiment of FIG. 14A, the laser source 620 can be surrounded by a reflector 626 (which can be a high reflector HR) and can output EMR into an output coupler 628 (OC).

The embodiment of the invention of FIG. 14B includes a phase mask 640 that is located between the output coupler 628 and an optical element 642. The phase mask 640 can include a set of apertures that spatially modulate the EMR. Various types of phase masks can be used in order to spatially modulate the EMR in order to form islets of treatment on the patient's skin 632. The optical element 642 can be any suitable element or an array of elements (such as lenses or micro lenses) that focuses the EMR radiation onto the patient's skin 632. In embodiment of FIG. 14B, the optical element 642 is a lens.

In operation, the laser source 620 generates EMR and the reflector 626 reflects some of it back toward the output coupler 628. The EMR then passes through the output coupler 628 to the phase mask 640, which spatially modulates the radiation. The optical element 642, which is optically downstream from the phase mask 640 so that it receives output EMR from the phase mask 640, generates an image of the apertures on the patient's skin.

FIG. 14C shows another embodiment of the invention. In this embodiment, the apparatus includes multiple laser sources 650 and optics to focus the EMR onto the patient's skin 632. The multiple laser sources 650 can be any suitable sources for this application, for example, diode lasers or fiber lasers. For example, the laser sources 650 can be a bundle of active rods made from garnet doped with rare earth ions. The laser sources 650 can optionally be surrounded by a reflector and/or an output coupler, similar to the embodiments of FIGS. 14A and 14B.

In the embodiment of FIG. 14C, an optical element 642 can be used for focusing the EMR onto the patient's skin 632. Any suitable element or an array of elements (such as lenses or micro lenses) can be used for the optical element 642. The optical element, for example, can be a lens 642.

In operation, the bundle of lasers 650 generate EMR. The EMR is spatially modulated by spacing apart the laser sources 650 as shown in FIG. 14C. The EMR that is output from the laser sources 650, therefore, is spatially modulated. This EMR passes through the output coupler 628 to the optical element 642, which focuses the EMR onto the patient's skin 632 to form islets of treatment.

In the exemplary embodiment of FIGS. 14A, 14B, and 14C, which each use a garnet laser rod doped with rare earth ions, the spectral range of electromagnetic radiation is about 400-3000 nm, the energy exposure is up to about 1000 J/cm², the laser pulse duration is from about 10 ps to 10 s, and the fill factor is from about 1% to 90%.

H. Consumer-Oriented Products and Methods

In another aspect, the invention can involve creating many zones of increased permeability in the SC without causing irreversible structural damage, or minimizing such damage, to the tissue. Reversible permeability is achieved by creating permeability of a topical in the SC for a limited time. Generally, this limited time corresponds to the application of EMR energy. After application of the EMR energy, the SC closes. Alternatively, permeability may remain for a period of time after application of the EMR energy. The time for permeability should be achieved in a limited time to prevent risk of infection. Using the principles of the present invention, such treatment can be made safe and painless, and thus can be practiced, for example, by members of general public, i.e., individuals with no special training. One such use is for enhancing the delivery of topical cosmetic compositions or pharmaceutical agents during in-home application.

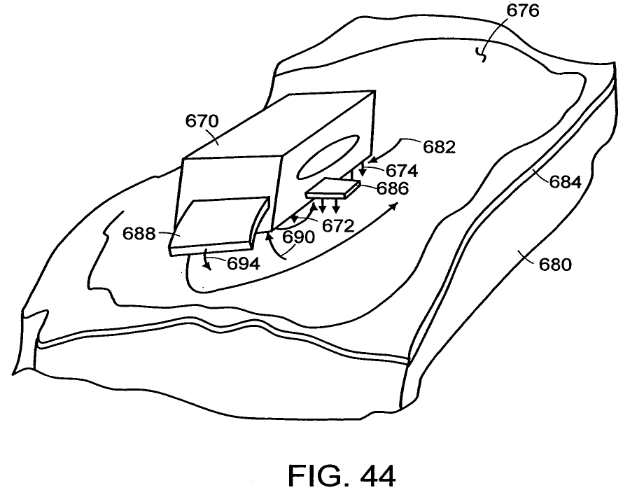

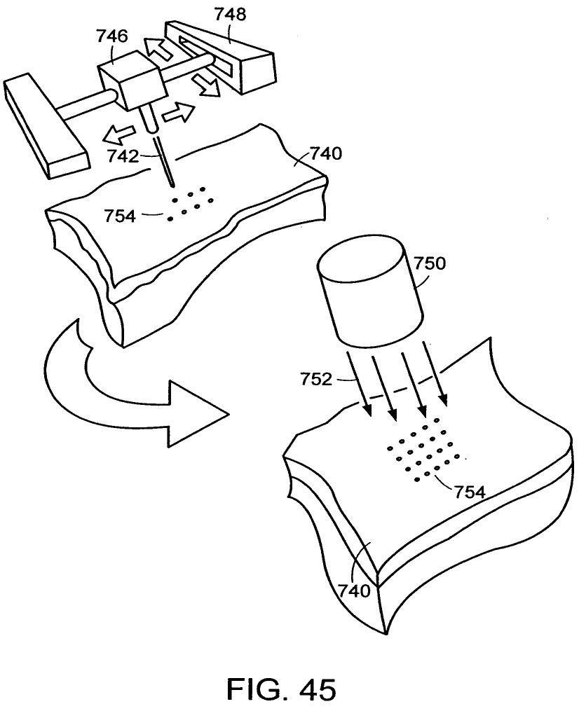

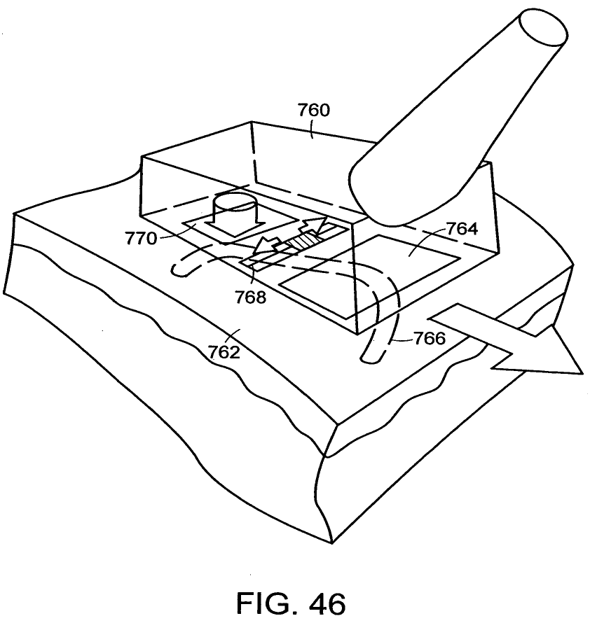

FIG. 44 is a schematic of a hand piece 670 according to this embodiment of the invention. In one example, the hand piece 670 emits a pattern 672 of beamlets 674 that irradiate the surface 676 of the skin 680. It creates thermal zones, e.g., moderate hyperthermia, in the skin to thereby create temporary permeability paths 682. The temporary permeability paths 682 can be created by inducing a series of phase transitions in the intercellular lipids connecting corneocytes of the stratum corneum layer 684. Lipids in the SC start to melt at about 35 C and completely melt at about 85 C. The hand piece 670 can also include a vibrator for skin massaging and/or an ultrasound or iontophoresis enhancer of permeability.

The hand piece 670 in one example uses an internal array of waveguides or an array of light emitting diodes (LED) or laser diodes to create the beamlet pattern 672. Suitable examples of LEDs or laser diodes are set forth above in connection with other embodiments. For example, a one-dimensional array of diode lasers or a stack of light emitting diode bars can be used. Numerous other types of EMR sources can also be used in this embodiment. In some embodiments, hand piece 670 can include multiple light sources for topical photo activation inside skin. In some embodiments, the wavelength of light is selected so that the skin is not damaged, but the SC become permeable for a limited period of time.

For controlled heating of the SC, endogenous or exogenous chromophores can used. For endogenous chromophores, water, lipids or proteins can be used. In one example, the spectrum of the light is in the range of or around the absorption peaks for water. These include, for example, 970 nm, 1200 nm, 1470 nm, 1900 nm, 2940 nm, and/or any wavelength>1800 nm. In other examples, the spectrum is tuned close to the absorption peaks for lipids, such as 0.92 μm, 1.2 μm, 1.7 μm, and/or 2.3 μm, and wavelengths like 3.4 μm, and longer for absorption peaks for proteins, such as keratin, or other endogenous tissue chromophores contained in the SC.

As a result of the phase transitions, balance between solid and liquid phases of lipids shifts towards the latter. This, in turn, leads to the development of enhanced permeability paths (not pictured) through the SC. Molecules, molecular complexes, or particles of a topical composition 694 may be discharged from (or through) an applicator 688 of the hand piece 670 or applied directly to the skin and penetrate through the paths 682 into the epidermis and dermis due to enhanced diffusion. The topical composition can be applied to the skin before, during, or after EMR treatment corresponding to the time that the SC has enhanced permeability.

In some embodiments, the bottom plate 690 of the hand piece 670 is cooled to increase skin safety and comfort as well as to accelerate restoration of the normal permeability of the SC after having delivered the composition. In other embodiments, the plate 690 is heated to facilitate the process of the pathway creation. Additional topical compound can be used after treatment to accelerate healing of SC after treatment.

In some embodiments, the thermal regimen can be reversed. For example, the hand piece 670 can create zones of hypothermia at the skin surface 676 in order to initiate the process of “freezing” of lipids in the SC. As a result, the lipid component shrinks and paths of facilitated percolation can be created. The formula above still holds, with minimal allowable temperature at the basal membrane approximately 15-18 C. The plate in such an embodiment can be heated for better skin protection and speedy restoration of the permeability.

This concept can be used for temporal delivery of cosmetic compounds into the skin, preferable into the epidermis. The compound can be removed from the skin with the growth of the epidermis. In addition, the compound can used for skin whitening or darkening, better scattering, and tattooing.

### EXAMPLE 5

Thermal Permeation of the Stratum Corneum

Lattices of thermal islets can be used to increase the permeability of the stratum corneum layer in a variety of ways, and to varying degrees, in accordance with the invention.

At temperatures in the range of 35-40° C., the outer most layers of the skin are subject to “soft hyperthermia” which is sufficient to increase the diffusion of some compounds into and through the stratum corneum and stratum lucidum. The permeability increases with “moderate hyperthermia” at temperatures in the range of 40-50° C. These temperatures are sufficient to initiate a phase change which partially melts or liquefies the typically crystalline lipid intercellular matrix of the stratum corneum and stratum lucidum. Generally, changes induced by this moderate heating, however, are reversible. After the heat source is removed, the lipid intercellular matrix recrystallizes with little or no permanent change. At temperature in the range of 50-100° C., the skin is subjected to “strong hyperthermia” which causes modification of the structure of the stratum corneum and stratum lucidum that is only partially reversible. By 85° C., lipid intercellular matrix is completely liquefied. the Heating the stratum corneum to temperatures of 100-200° C. causes evaporation of water and induces irreversible disruption of the stratum corneum to form micro gaps, but does not remove the stratum corneum. Rapid heating of the stratum corneum to temperatures greater than 200° C. causes denaturation of the proteins of horny cells and vaporization of the lipids or water of the stratum corneum structure. The resulting pressure waves from the vaporization can create holes in the stratum corneum.

Moderate and strong hyperthermia typically induce a pain response in a subject. Generally, the sensory nerves in the papillary dermis serve to sense and transmit heat, pain, and other noxious sensations. When exposed to temperatures in excess of 40-43° C., these sensory nerves will transmit a pain response in most subjects. Thus, moderate and strong hyperthermia typically require at least local anesthesia if applied uniformly or continuously on the skin surface. The local anesthesia can be achieved, for example, either by using topical formulations (e.g., lidocaine, LMX4™, Ferndale Laboratories, Inc., Ferndale, Mich.) or by pre-cooling the treatment area in order to decrease the sensitivity of the skin.

1. EMR-Absorbing Particles

In some embodiments, the invention provides a film with a lattice of EMR-absorbing particles in the form of dots, lines or other shapes, either on the surface of the film or embedded within the film. The EMR-absorbing particle arrangement can be random, or can have a regular pattern, such as a grid structure. For example, the material of the film can be a transparent, temperature-stable, preferably flexible composition with low thermal conductivity, such as an optically clear polymer; whereas the material of the EMR-absorbing particles is a substance, such as carbon, ink, or metal, which is appropriate to the EMR source. The EMR-absorbing particles can be spheres with diameters of 1-1000 μm, typically 50-500 μm. The spheres can be packed into the film with a fill factor of about 1-100%. For higher fill factors, such as about 50-100%, the film plays the additional role of protecting the skin from light. The size of a resulting thermal islet on the skin can be smaller or larger than an EMR-absorbing particle depending on particle temperature, degree of contact of the particle with the skin, and the presence of other substances (e.g., oil, lotion, vaseline) with appropriate thermal properties at the particle/skin interface that may help to conduct heat away or keep the heat of the particle confined to the particle/skin interface.

In some embodiments, the film can include additional waveguides on top of the EMR-absorbing particles. In certain embodiments, the waveguides can be cone-shaped. The purpose of the waveguides is to provide additional concentration of EMR energy into the islets. This can be achieved, for example, by using a transparent material with a refractive index higher than that of the film, and utilizing the phenomenon of the total internal reflection (TIR).

In another aspect of the invention, the film or the EMR-absorbing particles of the film can be impregnated with a cosmetic or therapeutic agent to be delivered through the stratum corneum. In these embodiments, the EMR-absorbing particles contain cavities which are filled with the agent intended for delivery, and have openings oriented towards the skin surface. Initially, the openings are closed by plugs to prevent leakage of the agent. When EMR energy is applied, the EMR-absorbing particles are heated and produce thermal islets with increased permeability in the skin. The material of the plugs is selected such that it is melted by the temperature increase, allowing the release of the agent to the thermal islets. In addition, in some embodiments, the contents of the particles can expand and form a series of jet-like streams directed toward the skin.

In one specific embodiment, a film with a pattern of carbon dots is employed. The carbon dots can be embedded in the film, or can be transferred from the film onto the skin and the film removed. For example, the carbon dots can be transferred by a first laser pulse, and then the dots on the skin can be irradiated by a second laser pulse or by irradiation from another source.

In some embodiments, the plurality of EMR-absorbing particles is exposed to EMR in the form of a uniform incident optical beam. Such a beam can be generated by, for example, a laser or flash lamp. The exposed particles absorb the radiation and release it as heat into the underlying areas of the stratum corneum, increasing the permeability of the stratum corneum and creating enhanced permeability paths for delivery of the agent.

The wavelength(s) of EMR used for exposure of the EMR-absorbing particles can be important. For example, the wavelength(s) can be in the range of approximately 290 nm to approximately 1000 μm. Generally, the wavelength(s) can be poorly absorbed in the body, particularly the skin, but well absorbed by the EMR-absorbing particles. The ratio of the absorption coefficient of the EMR-absorbing particles to the absorption coefficient of skin should be greater than 1. Thus, when irradiated, the EMR-absorbing particles will be preferentially heated and will transfer heat to the stratum corneum layer of the underlying skin. In contrast, EMR that does not strike the particles will not be absorbed efficiently by the skin and, in addition, the resulting heat will be distributed over a large depth profile within the skin, resulting in only diffuse heating, avoiding significant local heating and damage to the skin or other structures.

In some embodiments, the incident fluence is in the range of 1 mJ/cm² to 1000 J/cm². If highly absorbing particles are used, typically 1 mJ/cm² is required per 20° C. of heating of the stratum corneum.

In some embodiments, the incident radiation can be applied in a pulsed fashion to minimize damage to the epidermis and dermis. The effective heating pulse width should be less than 100 times the thermal relaxation time of the islets. Thus, pulse widths are typically in the range of 100 femto seconds to 1 second, depending on the islet size that is selected.

In addition to the use of films, as set forth above, the invention can be practiced by providing a topical composition that includes EMR-absorbing particles (e.g., chromophores) in a liquid carrier, such as a solution, suspension, cream or lotion. The topical composition can be applied to the skin, resulting in a random distribution of the EMR-absorbing particles on the surface. The density of the EMR-absorbing particles on the surface can be controlled by varying the concentration of the EMR-absorbing particles in the topical composition, or by varying the amount of the topical composition which is applied. Upon application of the EMR source, the EMR-absorbing particles can warm up, thus selectively producing thermal islets of treatment. Any of the variety of materials set forth above for EMR-absorbing particles can be used in the topical composition.

In another embodiment, a spatially selective pattern of EMR-treated islets can be created by applying to the skin surface a desired pattern of a topical composition containing a preferentially absorbing exogenous chromophore. First, a desired pattern of the composition is applied to the selected skin treatment area using a printing head mounted on a scanner. Next, an EMR delivery system delivers a beam of radiation to the treatment area, thus preferentially heating the composition. The resulting heat diffusion from the patterned chromophores of the composition yields a corresponding pattern of thermal islets. The dimensions of a thermal islet can, for example, vary between 1 μm and 3 mm, and the distance between the islets can, for example, vary between 1 and 1000 times their dimensions.

In another embodiment, instead of applying the topical composition directly to the skin surface, the composition can be applied first to an EMR-transparent film. Then, the film can be applied to the skin surface, and the radiation can be delivered through the film. The spectral composition of the incident radiation should match the absorption spectrum of the chromophore. Any of a variety of substances can be used as chromophores in this embodiment including, but not limited to, carbon, metals (e.g., Au, Ag, Fe), organic dyes (e.g., methylene blue, toluidine blue, trypan blue), non-organic pigments, and fullerenes. Fluences of the radiation can, for example, range from 0.1 to 1000 J/cm², and pulse width can, for example, range from 1 ps to 10 sec. The desired pattern need not be regular or pre-determined. It can vary as a function of the skin condition at the desired treatment area or be generated ad hoc by the physician or technician.

In another embodiment, all of the features described with respect to a film can be implemented at the distal end of a light source which is contacted to the skin.

In another embodiment, the hand piece of an EMR source can be scanned along the skin surface. A tracking/imaging device (e.g., digital camera or capacitance array) in the hand piece can detect, segment, and follow target volumes (e.g., pigmented lesions or vascular abnormalities). An EMR-absorbing particle (e.g., chromophore) dispenser in the hand piece can dispense the EMR-absorbing particles according to the tracking information, following projection of the target on the skin surface. The EMR source can then irradiate the EMR-absorbing particles dispensed in the treatment area.

Another embodiment is a dermatological delivery device that includes a substrate. According to this embodiment, the substrate has a plurality of absorbing elements, such as those set forth above, and a composition contained on at least one side of the substrate. Incident radiation from an energy source can heat up the absorbing elements so that the absorbing elements create treatment islets in the stratum corneum of a person's skin. After removal of the substrate, at least a substantial portion of the composition remains on the person's skin. That is, the composition, which can be cosmetic, therapeutic, or medical, can be attached to or disposed within or on the substrate in a manner so that at least some meaningful portion of the composition remains on the skin when the substrate is removed.

When the goal of treatment is to facilitate penetration of a cosmetic or therapeutic agent, the tracking/imaging device can be replaced with a dispenser for the agent.

2. Exothermic Compounds

In other embodiments, a film is employed which includes particles of an exothermic compound, and the particles are held in close proximity to or deposited onto the skin surface.

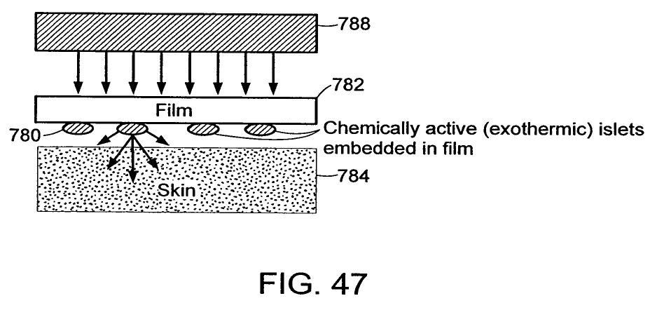

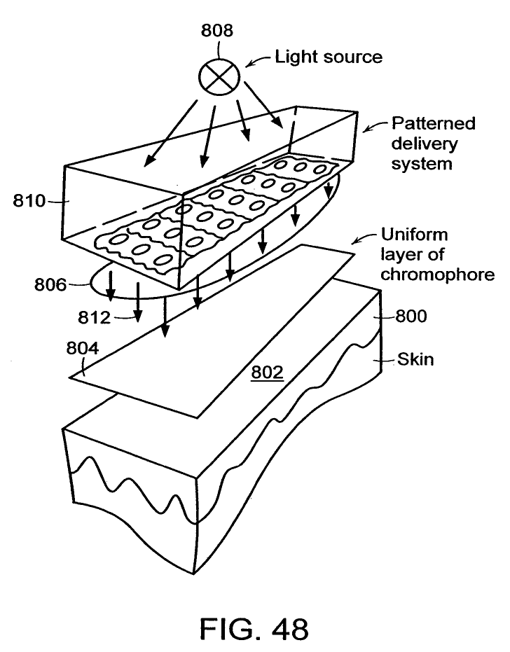

In some embodiments, small volumes of the exothermic compound 780 are attached to or embedded in a film 782 or other carrier, as shown in FIG. 47. Application of this film 782 to the surface of the skin 784 holds the compound in a heat conductive relationship with the skin 784. In certain embodiments, light or electrical discharge (as shown originating from light source 788) is used to ignite (initiate) a reaction of the exothermic compound, which leads to a controlled release of the chemical energy into the underlying stratum corneum. For example, a mixture of a light-absorbing chromophore (e.g., carbon) with an exothermic reagent (e.g., nitroglycerin) can be used. The chromophore absorbs the energy, and releases it as heat that ignites the exothermic reagent.

3. Patterned Radiation

In other embodiments, a continuous or mostly continuous coating of an EMR-absorbing compound is applied to the skin. For example, carbon paper, dye solution, or a thin layer of an EMR-absorbing lotion can be used. The EMR source must have a spectrum that matches the absorption peaks of the EMR-absorbing compound. For example, if water is used as the EMR-absorbing compound, the spectrum can include wavelengths of approximately 1.45, 1.9 and >2.3 μm.

The continuous coating is then exposed to a pattern of EMR. The EMR pattern can be produced using a source of uniform radiation, such as a laser or flash lamp, and an amplitude or phase mask or other delivery system for producing optical islets or beamlets of the pattern. Alternatively, the beamlets can be produced through multiple sources, such as multiple diode laser emitters or fiber bundles, for example. The beamlets locally heat the EMR-absorbing compound (e.g., chromophore) coating, which then creates thermal islets.

In another example, an interference pattern (e.g., Moire pattern) is created by a source at the skin surface. The patterns are designed such that the intensity at the nodes, or regions of constructive interference, exceeds a threshold for creating the permeability paths through the stratum corneum whereas the intensity between the nodes remains below the threshold.

In a particular embodiment, the patterned radiation can be a periodic lattice. The parameters of the patterned radiation are controlled by selecting the geometry of the incident beam, source settings, and properties of the EMR-absorbing compound, as well as its concentration.

### EXAMPLE 6

Rapid Acne Treatment Device

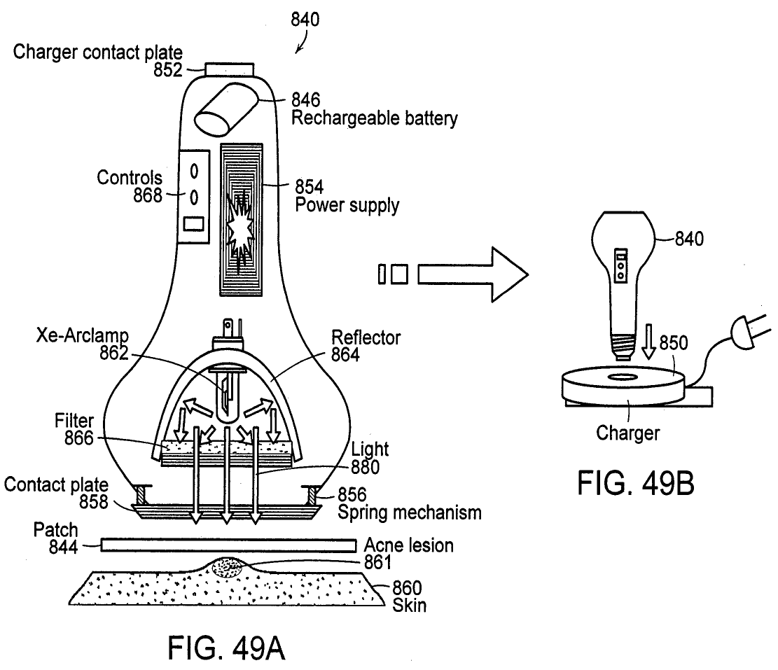

Another embodiment of the invention is shown in FIGS. 49A-B, 50, and 51A-B. The purpose of the device of this embodiment is rapid reduction of volume and redness of inflammatory acne lesions (single lesion treatment). For example, the reduction in redness and inflammation may occur within about 8-12 hrs. Although these embodiments are described for use in acne treatment, there are other possible uses of these embodiments as well.

A. Acne Treatment Device with Bulk Output

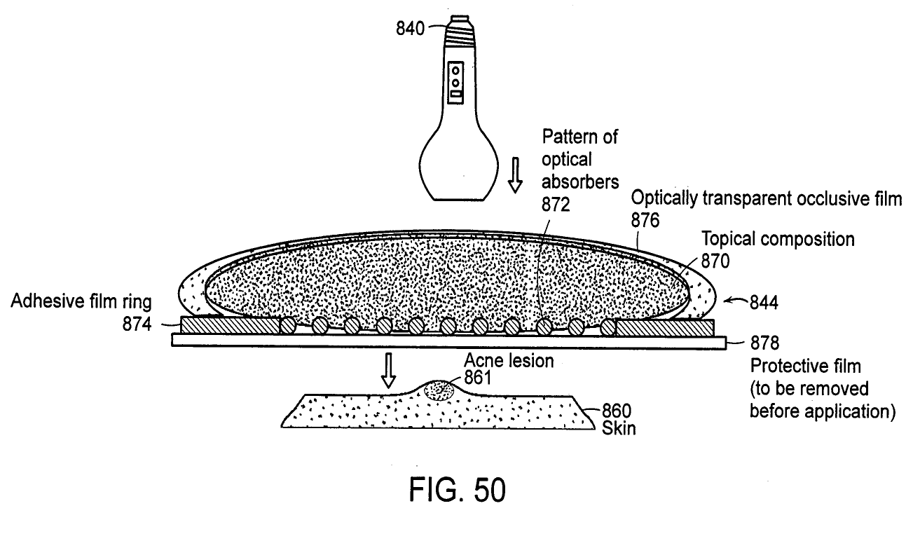

FIGS. 49A, 49B, and 50 show one embodiment of the acne treatment device. In this embodiment, the primary role of the light 842 is to facilitate delivery of a topical medication through the stratum corneum without seriously compromising the skin barrier function. Optionally, the light can also provide an additional benefit in mitigating the acne, independent of the topical medication. The system in this embodiment includes an applicator 840 to deliver light and a patch 844, which can contain a topical medication and which can also heat upon exposure to light, facilitating penetration of the stratum corneum. This medication can be result in vascular contraction, an anti-inflammatory effect, and reduction of bacterial. It can also be medication with a PDT effect.

In this embodiment, the device is a pulsed-light system, implemented as a hand-held cordless applicator 840 and a charger 850. In this embodiment, the applicator 840 can be a stand-alone device. In other embodiments, the applicator 840 can be attached to a base unit through an umbilical cord. The applicator 840 includes a rechargeable battery 846 that stores energy sufficient for a number of optical pulses, such as, for example, up to 15 optical pulses. A charger contact plate 852 on the applicator 840 engages with the charger 850 in order to recharge the rechargeable battery 846 (see FIG. 49B). The applicator 840 can also include a power supply 854.

The applicator 840 can also include a spring 856 and a contact plate 858, which together form a spring-loaded contact plate. The spring-loaded contact plate can ensure a controlled mechanical pressure of the contact plate 858 on the patient's skin 860. In addition, the spring-loaded contact plate can form a system that enables light output from the applicator 840 only when the plate 858 is in contact with patient's skin 860. For example, a sensor or the like can be included in the applicator 840 to sense when the contact plate 858 is in contact with the skin 860, and the applicator 840 can disable the light source of the applicator 840 when the contact plate 858 is not in contact with the patient's skin 860. The contact plate 858 can be made from a transparent material such as, for example, sapphire. The contact plate 858 can have other features similar to other contact plates described in this specification (such as, for example, cooling features).

In the embodiment of FIGS. 49A, 49B, and 50, the applicator 840 includes an EMR source 862 and optionally, a reflector 864 and a filter 866. The reflector 864 and filter 866, if used, can be the same as or similar to those set forth in the embodiments above. The EMR source 862 can be, for example, a Xe-flashlamp-based device, as shown in FIG. 49A. Other EMR sources 862 can be used in other embodiments. The applicator 840 can also include controls 868 to control the fluence of the light, the filtering of the light through filter 866, and other functions.

The system also includes a patch 844. The role of the patch 844 is two-fold. First, it serves as a container for the topical composition 870 for application through the skin 860. Second, it can feature a highly absorptive optical pattern 872, realized either as a net or as a set of separate “islands” (such as dots). FIG. 50 shows an enlarged patch 844 (the applicator 840 is not to scale in FIG. 50). Referring to FIG. 50, the patch 844 contains a topical medication 870, an adhesive ring 874, an external occlusive film 876, a pattern of optical absorbers 872, and a protective film 878. The topical medication 870 can be any compound, composition, or medicine intended for delivery through the skin 860. For instance, it can be a compound to treat acne.

The pattern of optical absorbers 872 can be made out of inert and biocompatible material to ensure a high absorption of light energy. For example, the optical absorbers 872 can be made from carbon powder. Numerous other types of optical absorbers 872 can also be used in place of or in addition to carbon powder. The pattern of the optical absorbers 872 can vary in different embodiments. In some embodiments, an organized matrix arrangement can be used, while in other embodiments, a less organized or random arrangement of optical absorbers 872 can be used.

The adhesive ring 874 is formed at the bottom of the patch 180 and is used for securing the patch 844 to the skin 860. The adhesive ring 874 can be shaped as a ring with an opening in the middle, although other geometries can also be used. The opening can prevent the adhesive ring 874 from interfering with the operation of the patch 844. That is, the opening will contact the skin 860 and not the adhesive ring 874, preventing the adhesive ring 874 from obstructing in the functionality of the patch 844. The adhesive ring 874 can be made from any adhesive material. In addition, although this application uses the term adhesive ring, any device for attaching the patch 844 to the skin surface 860 can be used in place of the adhesive ring 874. The protective film 878 covers the bottom of the patch 844 prior to use and is intended to be removed before application. The protective film 878 serves to keep the adhesive ring 874 fresh prior to use and to protect the rest of the patch 180 from contamination. The occlusive film 876 is generally transparent to light 880 and serves to protect the top of the patch 844.

In operation, the patch 844 is brought into contact with the skin 860. In an embodiment for treating acne, the patch 844 can be placed over a portion of skin 860 with an acne lesion 861. The user can then use the applicator 840 to deliver pulses of light 880 to the patch 844. When a pulse of light 880 shines on the patch 844, the optical absorbers 872 absorb the light energy, which results in a rapid temperature elevation. Since the optical absorbers 872 contact the skin surface 860, some of the thermal energy will be conducted to the stratum corneum, creating a corresponding pattern of enhanced permeability channels in the stratum corneum. Thus, penetration of the topical medication 870 into the skin 860 is accelerated, enabling faster effect of the medication. The patch 844 is then left on the skin 860 for a short period of time, for example, up to about two hours. Parameters of the light/patch system are selected in such a way so that no irreversible damage is caused to the stratum corneum; that is, so that integrity of the skin barrier is restored in a short time. The expected benefit is a more rapid improvement in the appearance of the acne lesion or other application.

B. Acne Treatment Device with Spatially Modulated Output

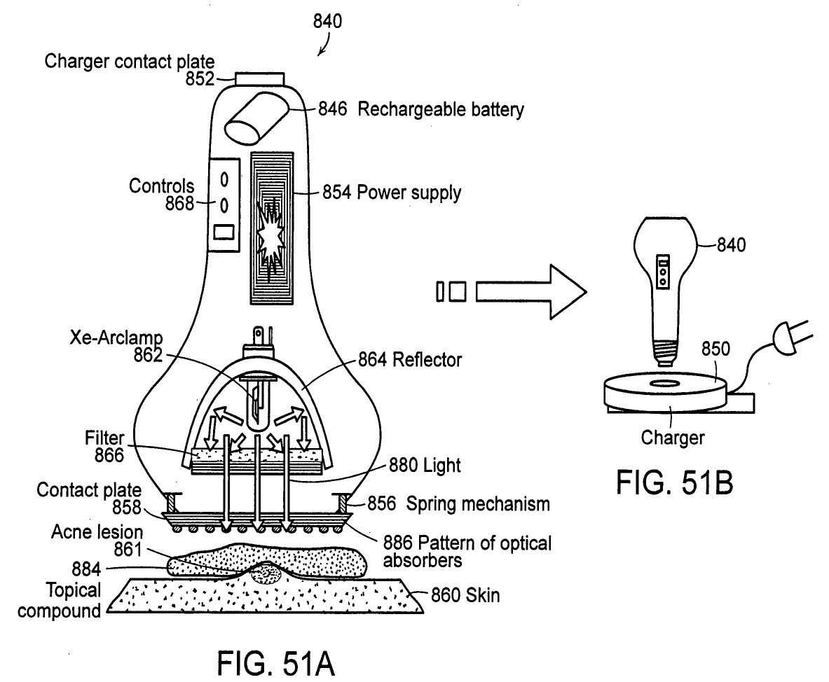

FIGS. 51A and 51B show a second embodiment of a treatment device for acne (or possibly other conditions). The device is similar to that described above, but the pattern of optical absorbers is created on the output plate of the device, rather than on a separate patch. In this embodiment, no patch is needed, and the topical composition can be applied directly to the skin 860.

Referring to FIG. 51A, the applicator 840 of this embodiment can include many of the same components as the embodiment of FIGS. 49 and 50. For example, the applicator 840 can include a rechargeable battery 846, a charge contact plate 852, a power supply 854, an EMR source 862, a reflector 864, a filter 866, and a contact plate 858 and spring 856. The charge contact plate 852 provides for engagement of the applicator 840 with a charger 850 so that the applicator 840 can be recharged. Each of these components can be the same or similar to those set forth above.

In the embodiment of FIG. 51A, the contact plate 858 contains a pattern of optical absorbers 886. In this embodiment, the pattern of optical absorbers 886 is robust enough to withstand multiple treatments. The optical absorbers 886 can be made from any suitable absorbing material, such as, for example, carbon powder. The pattern of optical absorbers 886 can be integrated in the contact plate 858 so that if the optical absorbers 886 are heated up, this heat can warm the skin 860.

In operation, the user can apply a topical compound 884 or medication to the skin 860 over an acne lesion 861. The user can place the contact plate 858 of the applicator 840 in contact with the patient's skin 860 and then use the applicator 840 to deliver pulses of light 880 to the optical absorbers 886. The optical absorbers 886 heat up, creating a pattern of enhanced permeability channels in the stratum corneum. Alternatively, the topical compound 884 can be applied after creation of the pattern of enhanced permeability channels in the stratum corneum.

In an embodiment using a flash lamp, the technical specifications of the treatment devices of FIGS. 49 through 51 can be as summarized in Table E1 below. These embodiments can be used for a number of applications, including skin diseases and cosmetic treatments.

TABLE E1

| Specification | Symbol | Value | Units |
|---|---|---|---|
| Incident Fluence | Finc | 1-25 | J/cm² |
| Wavelength Range (of EMR | λₘᵢₙ, λₘₐₓ | 400-2000 | nm |
| source) | | | |
| Spot Size (of optical absorbers) | SS | 1-50 dia. | mm |
| Pulse width (of EMR source) | PW | 1-1000 | ms |
| Lifetime | Tlife | 10-10000 | pulses |
| Number of Lamps (of EMR | #lamps | 1-10 | # |
| source) | | | |
| Pulse Period (of EMR source) | T | 1-10 | sec |
| Island/mesh Diameter | ID | 10-100 | um |
| Pattern pitch | PP | 100-5000 | um |

### EXAMPLE 7

### Treatment of Deep Layer of Tissue

The present invention provides means for creating non-uniform (modulated) temperature profiles (MTP) deep in the dermis and in hypodermis (typically, at depths exceeding 500 μm). In some embodiments, such profiles result in formation of a pattern (lattice) of islets of damage (LID). Active or passive cooling is applied to epidermal surface in order to prevent epidermal damage. Thus, the technique of the present invention combines advantageous features of non-ablative and fractional techniques. Creation of MTPs leads to improvements in skin structure and texture via the following mechanisms (the list is not exclusive):

1. Lifting and tightening of skin as a result of shrinkage of collagen fibrils subjected to elevated temperature (immediate effect).

2. Lifting and tightening of skin as a result of coagulation of localized areas in the dermis an(i hypodermis (immediate to short-term effect).

3. Improvement in skin texture as a result of coagulation of localized areas in the dermis and hypodermis (immediate to short-term effect).

4. Promotion of collagen production due to healing response to thermal stress and/or thermal shock (medium- to long-term effect).

A number of other local and systemic pathologies can be treated with the technique:

1. Acne. By selecting the wavelength of the optical radiation to promote preferential absorption of the optical energy by sebum and/or organizing the pattern to target preferentially sebaceous glands, selective destruction of the glands can be achieved.

2. Hypertrophic scars. By inducing tightening and shrinkage in the scar tissue, transformation of the abnormal connective tissue to normal one can be initiated.

3. Odor reduction. By selectively targeting eccrine glands, production of eccrine sweat can be reduced, and its composition can be changed.

4. Non-skin-surface texturing. The technique can be used for organ augmentation (e.g., lips).

5. Cellulite. By changing mechanical stress distribution at the dermis/hypodermis border, the appearance of cellulite can be improved.

It appears that the angular profile in skin is neither Gaussian nor Lambertian. In fact it is close to uniform. In further considerations we used the Gaussian angular profile 90 deg in full width (1/e² level). The transverse intensity profile was assumed to be flat.

A source with the blackbody spectrum at 3000 K as halogen lamp, the skin is of type II were simulated. The heat production at the specified depth is normalized to the input light flux so that the resultant value is expressed in 1/cm. The pass bands are 0.9-1.3; 0.9-1.5; 0.9-1.8 μm. The depths in tissue are 2 and 3 mm. Therefore, we have 6 variants.

Damage Profiles.

To evaluate the damage profiles the following model was used: The monochromatic light strikes the skin of type II through sapphire plate 5 mm in thickness. The initial plate temperature is 0 C. The plate surface opposite to the skin is held at fixed temperature 0 C. The light is monochromatic. There are 3 steps: precooling, light treatment, and post cooling. The sapphire plate with dielectric mirror type coating with transparent holes is held in contact with the skin all the time. The irradiance distribution is evaluated using the MC routine, then, the irradiance data are used to evaluate the temperature and damage dynamics. The beam is 7 mm in diameter and the full angle of divergence is 90 deg in the skin.

Under the reasonable choice of the input fluence the damage zone is 1-6 mm in diameter that is smaller than the beam diameter. For the 10 s treatment time the depth of the damage zone is 2-2.8 mm (1064 nm), about 2 mm (1270 nm), about 1.5 mm (1700 nm), 1.1-1.2 mm (1560 nm) depending on the treatment time. (The larger is the treatment time the deeper is the damage zone). The characteristics of the damage zone are almost independent on the precooling and post cooling times. When using collimated beam instead of divergent one the light flux may be slightly decreased, however, the location and the shape of the damage zone does not change appreciably. The damage zone is almost spherical for 1064 and 1270 nm and becomes squeezed in the vertical direction for 1700 and 1560 nm. It appears that the distance between the spots has to exceed the spot diameter by at least 1.5 mm.

Experimental Results

A tungsten halogen lamp-based device with appropriate filters provides output radiation between 800 nm and 3.0 mm at adjustable fluences and pulse widths from 1 to 15 J/cm². This device also has a cooled sapphire window interface through which the radiation is applied that contacts with the sample tissue. The beam diameter is fixed at 8 mm. Full thickness, farm pig skin is prepared and placed on a heated pad to provide approximate temperatures of 35 degrees C. at the bottom layer (fat and sub-dermis) with a surface temperature approximately 30 degrees C. The sapphire window is cooled to approximately 10 degrees C. via water cooling lines and a chiller. In one experiment, the device is place in contact with the pig skin for a prescribed precooling period prior to turning on the lamp for treatment.

FIGS. 51(a-c) demonstrate skin tightening without epidermis damage. A single treatment exposure is then applied in succession to each of the upper-left four squares (FIG. 51b) followed by a treatment to the lower-left four squares (FIG. 51c).

There is a clear distortion of the skin surface (seen by the distortion of the grid lines) that suggests shrinkage as a result of the treatment. LDH staining reveals the extent of thermal damage to the tissue in FIG. 52. The damaged zones span 4-5 mm and are 1 mm in thickness just below the epidermal layer. Note that the epidermis is not damaged by the treatment.

Equivalents.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize, or be able to ascertain using no more than routine experimentation, many equivalents to the specific embodiments of the invention described specifically herein. Such equivalents are intended to be encompassed in the scope of the appended claims.

TABLE AA1
Layer parameters. The OD values apply to the reference wavelength 800 nm.

| | | | OD of | | | | | Scat, | | | |
|---|---|---|---|---|---|---|---|---|---|---|---|
| | | | melanin | | Water | Blood | Mean vessel | coef. at | | Specific | Thermal |
| | Thickness | Refraction | (skin types | | content, | content, | diameter | 577 nm | Density | heat, | conductivity, |
| Layer | (μm) | index | I-VI) | | (%) | (%) | (μm) | (mm⁻¹⁾ | (g/cm³) | (J/g K) | W/(cm K) |
| Upper | 70 | 1.45 | I | 0.0035 | 60 | 0 | — | 30 | 1.12 | 3.05 | 0.00294 |
| | | | II | 0.0061 | | | | | | | |
| | | | III | 0.0087 | | | | | | | |
| | | | IV | 0.019 | | | | | | | |
| | | | V | 0.0491 | | | | | | | |
| | | | VI | 0.0952 | | | | | | | |
| Basal | 15 | 1.4 | I | 0.0081 | 60 | 0 | — | 30 | 1.12 | 3.05 | 0.00294 |
| | | | II | 0.0141 | | | | | | | |
| | | | II | 0.0202 | | | | | | | |
| | | | IV | 0.0444 | | | | | | | |
| | | | V | 0.0491 | | | | | | | |
| | | | VI | 0.0952 | | | | | | | |

| | | | | | | | | | | |
|---|---|---|---|---|---|---|---|---|---|---|
| Reticular derm. & | 200 | 1.38 | 0 | 75 | 1.7 | 6 | 12 | 1.075 | 3.50 | 0.00407 |
| plexus | | | | | | | | | | |
| Dermis | 3000 | 1.35 | 0 | 75 | 1.4 | 15 | 12 | 1.075 | 3.50 | 0.00407 |
| Sapphire window | 3000 | 1.76 | — | — | — | — | 0 | 3.97 | 0.419 | 0.2721 |

TABLE F1
Exemplary treatment parameters.

| | | | | |
|---|---|---|---|---|
| Damage | 1 | 2 | 3 | 5 |
| heating depth, | | | | |
| mm | | | | |
| Damage/heated | 0.2-3 | 0.5-5 | 0.75-6 | 1-10 |
| zone diameter, | | | | |
| mm | | | | |
| Wavelength, | 900-1850 | 900-1400 | 900-1350 | 900-1300 |
| nm | 2080-2300 | 1500-1750 | | |
| Beam diameter | 0.5-8 | 1-10 | 2-15 | 3-25 |
| (2D beam) or | | | | |
| width (1D | | | | |
| beam), mm | | | | |
| Fill factor* | 0.01-0.5 | 0.01-0.3 | 0.01-0.3 | 0.01-0.3 |
| Pulse width, s | 0.001-10 | 0.1-20 | 0.5-30 | 1-120 |
| Precooling | 0-10 | 0-20 | 0-60 | 0-100 |
| time, s | | | | |
| Postcooling | 0-20 | 0-30 | 0-60 | 0-120 |
| time, s | | | | |
| Input power | 5-100 | 3- 70 | 1 - 50 | 0.5 - 35 |
| density, W/cm² | | | | |

*Fₘₐₓ is the maximum possible fill factor, that is, the ratio
of the light exposed area the total area of the treatment
$F = \frac{π}{4} \cdot {( \frac{D}{d} )}^{2} ,$
where D is the spot diameter, d is the spot separation.

1-285. (canceled)

286. An apparatus for performing a treatment on tissue, comprising:
a housing having a portion that defines a target treatment area on the tissue when placed in proximity to the tissue; and
a light emitting bar for applying EMR to the target treatment area, wherein the light emitting bar includes multiple emitters of EMR for creating treatment islets in the tissue, each emitter creating a treatment islet.

287. The apparatus of claim 286, wherein, when in use, outputs from the emitters are within about 50 to 1000 microns of the tissue.

288. The apparatus of claim 286, wherein the apparatus does not have focusing optics between the light emitting bar and the portion of the housing that defines the target treatment area.

289. The apparatus of claim 286, wherein the apparatus does not have imaging optics between the light emitting bar and the portion of the housing that defines the target treatment area.

290. The apparatus of claim 286, wherein a distance between outputs from the emitters and an output from the housing is about 50 to 1000 microns.

291. The apparatus of claim 286, further comprising a contact plate disposed between the light emitting bar and the tissue when in use, wherein a distance between outputs from the emitters and an output from the contact plate is about 50 to 1000 microns.

292. The apparatus of claim 286, wherein the contact plate is part of the housing.

293. The apparatus of claim 286, wherein the contact plate is operably attached to the housing.

294. The apparatus of claim 286, wherein the light emitting bar is mounted within the housing.

295. The apparatus of claim 286, further comprising a base unit, wherein the light emitting bar is mounted within the base unit.

296. The apparatus of claim 286, wherein the emitters are spaced apart in a regular pattern.

297. The apparatus of claim 296, wherein the emitters are spaced apart by about 50 to 900 μm.

298. The apparatus of claim 297, wherein the emitters emit EMR in a wavelength range of about 290 to 10,000 nm.

299. The apparatus of claim 298, wherein the emitters emit EMR in a wavelength range of about 900 to 10,000 nm.

300. The apparatus of claim 297, wherein the emitters have widths of about 50 to 150 μm.

301. The apparatus of claim 286, wherein the light emitting bar is an LED bar.

302. The apparatus of claim 286, wherein the light emitting bar is a diode laser bar.

303. The apparatus of claim 302, wherein the emitters are spaced apart by about 50 to 900 μm.

304. The apparatus of claim 303, wherein the emitters have widths of about 50 to 150 μm.

305. The apparatus of claim 304, wherein the diode laser bar is about 1 cm long.

306. The apparatus of claim 305, wherein the diode laser bar is about 1 mm wide.

307. The apparatus of claim 306, wherein the diode laser bar includes 10 to 15 emitters.

308. The apparatus of claim 302, wherein the apparatus includes more than one diode laser bar.

309. The apparatus of claim 308, wherein the apparatus includes five or more diode laser bars.

310. The apparatus of claim 308, wherein the diode laser bars are formed as a stack to create a matrix of treatment islets in the tissue when in use.

311. The apparatus of claim 286, further comprising one or both of a cooling element or a heating element attached to the housing.

312. The apparatus of claim 311, wherein the element is disposed between the light emitting bar and the tissue when in use.

313. The apparatus of claim 311, wherein the element allows passage therethrough of at least a portion of the EMR from the light emitting bar.

314. The apparatus of claim 313, wherein the element provides cooling over an entire area of contact with the tissue.

315. The apparatus of claim 313, wherein the element creates islets of cooling between the treatment islets.

316. The apparatus of claim 286, wherein the portion of the housing that defines the target treatment area comprises one or both of a cooling element and heating element, wherein the element is disposed between the light emitting bar and the tissue when in use.

317. The apparatus of claim 286, further comprising a speed sensor to measure a speed of movement of the housing across the tissue.

318. The apparatus of claim 317, further comprising circuitry to vary the output power of the light emitting bar in response to the speed of movement of the housing across the tissue.

319. The apparatus of claim 318, wherein the circuitry regulates at least one of an interval between pulses and pulse duration of the diode laser bar so that at least one of the interval and the pulse duration is inversely proportional to the speed of movement of the housing across the patient's skin.

320. The apparatus of claim 317, wherein the speed sensor is one of a capacitive imaging array or an optical imaging array.

321. The apparatus of claim 320, wherein the one of the capacitive imaging array and the optical imaging array creates images of the target treatment area when in use.

322. The apparatus of claim 286, further comprising a translator to move the light emitting bar with respect to the housing.

323. The apparatus of claim 322, further comprising circuitry to control the motor to move the light emitting bar in a direction opposite to a direction of movement of the housing across the tissue.

324. The apparatus of claim 322, wherein the apparatus creates a matrix of treatment islets when the translator moves the light emitting bar with respect to the housing.

325. The apparatus of claim 286, further comprising one of an imaging, refractive or diffractive optical element disposed in the housing between the light emitting bar and the portion of the housing that defines the target treatment area.

326. The apparatus of claim 286, wherein the housing has a head portion that contacts the tissue when in use.

327. The apparatus of claim 326, further comprising a contact sensor to sense when the head portion is in contact with the tissue.

328. The apparatus of claim 286, further comprising a bundle of optical fibers, wherein each optical fiber is connected to at least one of the emitters.

329. The apparatus of claim 328, wherein each optical fiber is connected to one of the emitters.

330. The apparatus of claim 328, further comprising imaging optics associated with an output of each of the optical fibers.

331. The apparatus of claim 328, further comprising focusing optics associated with an output of each of the optical fibers.

332. The apparatus of claim 286, wherein the apparatus is a hand held device.

333. The apparatus of claim 286, further comprising an optical element operably coupled to each emitter, wherein the optical element aids in creating the treatment islets.

334. The apparatus of claim 286, further comprising a sensor to sense one or more of contact of the housing with the tissue, speed of moving of the housing over the tissue, and imaging of the tissue.

335. The apparatus of claim 286, further comprising a scanner to move the housing over the tissue during use.

336. The apparatus of claim 286, wherein the housing can be manually moved over the tissue during use.

337. An apparatus for performing a treatment on tissue, comprising:
a hand held piece having a portion that defines a target treatment area on the tissue when placed in proximity to the tissue;
a base unit containing a light emitting bar having multiple emitters of optical energy;
a bundle of optical fibers, wherein each optical fiber is connected to at least one of the emitters, wherein the multiple emitters and bundle of optical fibers create treatment islets in the tissue, each optical fiber creating a treatment islet in the tissue.

338. The apparatus of claim 337, wherein each optical fiber is coupled to one of the emitters.

339. The apparatus of claim 338, further comprising an imaging optical system in the hand held piece, wherein the imaging optical system includes imaging optics associated with an output of each of the optical fibers.

340. The apparatus of claim 338, further comprising a focusing optical system in the hand held piece, wherein the focusing optical system includes focusing optics associated with an output of each of the optical fibers.

341. A handheld dermatological device, comprising:
a housing capable of being manipulated to position the housing in proximity to tissue; and
a light emitting bar supported by the housing, the light emitting bar having multiple emitters of EMR, each emitter creating a treatment islet.

342. The handheld dermatological device of claim 341, further comprising at least one of a cooling or heating surface operably connected to the housing, wherein the surface is placed in proximity to the tissue during use to define a target treatment area on the tissue, and wherein output EMR from the light emitting bar passes through the surface to produce the treatment islets in the tissue.

343. The handheld dermatological device of claim 342, wherein the cooling or heating surface is a sapphire plate.

344. The handheld dermatological device of claim 342, further comprising an optical element disposed in an optical path between the light emitting bar and the surface for focusing the output EMR.

345. The handheld dermatological device of claim 342, wherein the emitters are spaced apart, with at least a portion of the target treatment area between the treatment islets not directly receiving EMR.

346. An apparatus for performing a treatment on a target treatment area of tissue, comprising a light emitting assembly for applying EMR to the target treatment area, the light emitting assembly including a light emitting bar with multiple emitters of EMR, wherein the EMR is applied in a plurality of sub-areas, with each emitter applying EMR in one of the sub-areas, at least a portion of the target treatment area between the sub-areas not directly receiving EMR.

347. The apparatus of claim 346, further comprising a heating element attached to the light emitting assembly, wherein the element is disposed between the light emitting assembly and the target treatment area of the tissue and is in contact with the target treatment area when the apparatus is in use to heat the target treatment area, the element allowing passage therethrough of at least a portion of the EMR from the light emitting assembly.

348. The apparatus of claim 346, further comprising a cooling element attached to the light emitting assembly, wherein the cooling element is disposed between the light emitting assembly and the target treatment area of the tissue and is in contact with the target treatment area when the apparatus is in use to dissipate heat from the target treatment area, the cooling element allowing passage therethrough of at least a portion of the EMR from the light emitting assembly.

349. The apparatus of claim 348, wherein the cooling element is made out of sapphire or diamond.

350. The apparatus of claim 348, wherein the cooling element is made out of a material that is optically transparent.

351. The apparatus of claim 346, wherein the light emitting bar is a diode laser bar, and wherein the light emitting assembly comprises multiple diode laser bars in a stacked arrangement.

352. The apparatus of claim 351, wherein the multiple diode laser bars emit radiation of different wavelengths.

353. The apparatus of claim 346, wherein the light emitting assembly is in a hand piece.

354. The apparatus of claim 346, wherein the light emitting assembly is in a separate base unit.

355. The apparatus of claim 346, wherein the light emitting assembly emits EMR in a wavelength range of about 290 to 10,000 nm.

356. The apparatus of claim 346, wherein, when in use, outputs from the emitters are within about 50 to 1000 microns of the tissue.

357. The apparatus of claim 346, wherein the light emitting assembly includes a housing for the light emitting bar, wherein the housing includes an output plate for placement in close proximity to the tissue when in use, wherein a distance between the outputs from the emitters and an output area of the output plate is about 50 to 1000 microns.

358. The apparatus of claim 346, wherein the light emitting assembly includes an element selected from the group consisting of an optical element and a mask.

359. A dermatological apparatus for performing a treatment on tissue, comprising:
means for supplying EMR to the tissue to create treatment islets, wherein the EMR supplying means includes a light emitting bar having multiple emitters of EMR; and
means for supporting the EMR supplying means, the means for supporting including a head portion capable of being manipulated to position the head portion in proximity to the tissue when in use.

360. The dermatological apparatus of claim 359, wherein the light emitting bar is selected from the group consisting of a diode laser bar and an LED bar.
